(19) United States
(12) Patent Application Publication
(10) Pub. No.: US 2022/0378742 A1
(43) Pub. Date: Dec. 1, 2022

(54) COMBINATION THERAPY WITH ANTI-PVRIG ANTIBODIES FORMULATIONS AND ANTI-PD-1 ANTIBODIES

(71) Applicants: Compugen Ltd., Holon (IL); Bristol-Myers Squibb Company, Princeton, NJ (US)

(72) Inventors: Adeboye Henry Adewoye, San Francisco, CA (US); John Hunter, Piedmont, CA (US); Anat Cohen-Dayag, Rehovot (IL); Paul A. Basciano, Princeton, NJ (US); Brian D. Lamon, Princeton, NJ (US)

(21) Appl. No.: 17/773,791

(22) PCT Filed: Jul. 28, 2020
(86) PCT No.: PCT/US20/43917

§ 371 Date: May 2, 2022

Related U.S. Application Data
(60) Provisional application No. 63/009,364, filed on Apr. 13, 2020.
(60) Provisional application No. 62/968,641, filed on Jan. 31, 2020.
(60) Provisional application No. 62/930,211, filed on Nov. 4, 2019.

(51) Int. Cl. A61K 31/41; A61K 38/16; A61K 47/18; A61K 47/22; A61K 9/08; A61P 35/00

(52) U.S. Cl. CPC A61K 31/41; A61K 38/162; A61K 47/183; A61K 47/22; A61K 9/08; A61P 35/00

(57) ABSTRACT

The present invention is directed to combination treatments with anti-PVRIG antibodies and anti-PD-1 antibodies, in particular nivolumab, using stable liquid pharmaceutical formulations thereof.

### CROSS-REFERENCE TO RELATED APPLICATIONS

This application claims priority to U.S. Provisional Patent Applications Nos. 62/930,211, filed Nov. 4, 2019, 62/968,641, filed Jan. 31, 2020 and 63/009,364, filed Apr. 13, 2020, all of which are incorporated by reference in their entireties.

### BACKGROUND OF THE INVENTION

Naïve T cells must receive two independent signals from antigen-presenting cells (APC) in order to become productively activated. The first, Signal 1, is antigen-specific and occurs when T cell antigen receptors encounter the appropriate antigen-MHC complex on the APC. The fate of the immune response is determined by a second, antigen-independent signal (Signal 2) which is delivered through a T cell costimulatory molecule that engages its APC-expressed ligand. This second signal could be either stimulatory (positive costimulation) or inhibitory (negative costimulation or coinhibition). In the absence of a costimulatory signal, or in the presence of a coinhibitory signal, T-cell activation is impaired or aborted, which may lead to a state of antigen-specific unresponsiveness (known as T-cell anergy), or may result in T-cell apoptotic death.

Costimulatory molecule pairs usually consist of ligands expressed on APCs and their cognate receptors expressed on T cells. The prototype ligand/receptor pairs of costimulatory molecules are B7/CD28 and CD40/CD40L. The B7 family consists of structurally related, cell-surface protein ligands, which may provide stimulatory or inhibitory input to an immune response. Members of the B7 family are structurally related, with the extracellular domain containing at least one variable or constant immunoglobulin domain.

Both positive and negative costimulatory signals play critical roles in the regulation of cell-mediated immune responses, and molecules that mediate these signals have proven to be effective targets for immunomodulation. Based on this knowledge, several therapeutic approaches that involve targeting of costimulatory molecules have been developed, and were shown to be useful for prevention and treatment of cancer by turning on, or preventing the turning off, of immune responses in cancer patients and for prevention and treatment of autoimmune diseases and inflammatory diseases, as well as rejection of allogenic transplantation, each by turning off uncontrolled immune responses, or by induction of “off signal” by negative costimulation (or coinhibition) in subjects with these pathological conditions.

Manipulation of the signals delivered by B7 ligands has shown potential in the treatment of autoimmunity, inflammatory diseases, and transplant rejection. Therapeutic strategies include blocking of costimulation using monoclonal antibodies to the ligand or to the receptor of a costimulatory pair, or using soluble fusion proteins composed of the costimulatory receptor that may bind and block its appropriate ligand. Another approach is induction of co-inhibition using soluble fusion protein of an inhibitory ligand. These approaches rely, at least partially, on the eventual deletion of auto- or allo-reactive T cells (which are responsible for the pathogenic processes in autoimmune diseases or transplantation, respectively), presumably because in the absence of costimulation (which induces cell survival genes) T cells become highly susceptible to induction of apoptosis. Thus, novel agents that are capable of modulating costimulatory signals, without compromising the immune system's ability to defend against pathogens, are highly advantageous for treatment and prevention of such pathological conditions.

Costimulatory pathways play an important role in tumor development. Interestingly, tumors have been shown to evade immune destruction by impeding T cell activation through inhibition of co-stimulatory factors in the B7-CD28 and TNF families, as well as by attracting regulatory T cells, which inhibit anti-tumor T cell responses (see Wang (2006), “Immune Suppression by Tumor Specific CD4⁺ Regulatory T cells in Cancer”, Semin. Cancer. Biol. 16:73-79; Greenwald, et al. (2005), “The B7 Family Revisited”, Ann. Rev. Immunol. 23:515-48; Watts (2005), “TNF/TNFR Family Members in Co-stimulation of T Cell Responses”, Ann. Rev. Immunol. 23:23-68; Sadum, et al., (2007) “Immune Signatures of Murine and Human Cancers Reveal Unique Mechanisms of Tumor Escape and New Targets for Cancer Immunotherapy”, Clin. Canc. Res. 13(13): 4016-4025). Such tumor expressed co-stimulatory molecules have become attractive cancer biomarkers and may serve as tumor-associated antigens (TAAs). Furthermore, costimulatory pathways have been identified as immunologic checkpoints that attenuate T cell dependent immune responses, both at the level of initiation and effector function within tumor metastases. As engineered cancer vaccines continue to improve, it is becoming clear that such immunologic checkpoints are a major barrier to the vaccines' ability to induce therapeutic anti-tumor responses. In that regard, costimulatory molecules can serve as adjuvants for active (vaccination) and passive (antibody-mediated) cancer immunotherapy, providing strategies to thwart immune tolerance and stimulate the immune system.

Over the past decade, agonists and/or antagonists to various costimulatory proteins have been developed for treating autoimmune diseases, graft rejection, allergy and cancer. For example, CTLA4-Ig (Abatacept, Orencia®) is approved for treatment of RA, mutated CTLA4-Ig (Belatacept, Nulojix®) for prevention of acute kidney transplant rejection and by the anti-CTLA4 antibody (Ipilimumab, Yervoy®), recently approved for the treatment of melanoma. Other costimulation regulators have been approved, such as the anti-PD-1 antibodies of Merck (Keytruda®) and BMS (Opdivo®), have been approved for cancer treatments and are in testing for viral infections as well.

A particular target of interest is PVRIG. PVRIG is a transmembrane domain protein of 326 amino acids in length, with a signal peptide (spanning from amino acid 1 to 40), an extracellular domain (spanning from amino acid 41 to 171), a transmembrane domain (spanning from amino acid 172 to 190) and a cytoplasmic domain (spanning from amino acid 191 to 326). The full length human PVRIG protein is shown in FIG. 1. There are two methionines that can be start codons, but the mature proteins are identical.

The PVRIG proteins contain an immunoglobulin (Ig) domain within the extracellular domain, which is a PVR-like Ig fold domain. The PVR-like Ig fold domain may be responsible for functional counterpart binding, by analogy to the other B7 family members. The PVR-like Ig fold domain of the extracellular domain includes one disulfide bond formed between intra domain cysteine residues, as is typical for this fold and may be important for structure-function. These cysteines are located at residues 22 and 93 (or 94). In one embodiment, there is provided a soluble fragment of PVRIG that can be used in testing of PVRIG antibodies. Included within the definition of PVRIG proteins are PVRIG ECD fragments, including know ECD fragments such as those described in U.S. Pat. No. 9,714,289.

PVRIG has also been identified as an inhibitory receptor which recognizes CD112 but not CD155, and it may be involved in negative regulation of the anti-tumor functions mediated by DNAM-1. PVRL2 was identified as the ligand for PVRIG, placing PVRIG in the DNAM/TIGIT immunoreceptor axis (see, Liang et al., Journal of Clinical Oncology 2017 35:15_suppl, 3074-3074).

Anti-PVRIG antibodies (including antigen-binding fragments) that both bind to PVRIG and prevent activation by PVRL2 (e.g. most commonly by blocking the interaction of PVRIG and PVLR2), are used to enhance T cell and/or NK cell activation and be used in treating diseases such as cancer and pathogen infection. As such, formulations for administering such antibodies are needed.

Accordingly, it is an object of the invention to provide stable liquid pharmaceutical formulations comprising anti-PVRIG antibodies or use in disease treatment (e.g., anti-PVRIG antibodies including those with CDRs identical to those shown in FIG. 3).

### BRIEF SUMMARY OF THE INVENTION

Accordingly, it is an object of the invention to provide methods of treatment comprising a combination of anti-PD-1 antibody and anti-PVRIG antibody, wherein the anti-PVRIG antibody is in a stable liquid pharmaceutical formulations of anti-PVRIG antibodies as described herein.

The present invention provides a method of treatment for cancer comprising administering nivolumab and an anti-PVRIG antibody, wherein the anti-PVRIG antibody is administered as a stable liquid pharmaceutical formulation and, wherein the stable liquid pharmaceutical formulation of the anti-PVRIG antibody comprises:

(a) an anti-PVRIG antibody, wherein said anti-PVRIG antibody comprises:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the v1CDR1, vlCDR2, and vlCDR3 from the light chain of CHA.7.518.1.H4(S241P) (SEQ ID NO: 9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments of the method of treatment said anti-PVRIG antibody comprises a CH1-hinge-CH2-CH3 sequence of IgG4 (SEQ ID NO:17 or SEQ ID NO:50), wherein said hinge region optionally comprises mutations.

In some embodiments of the method of treatment said anti-PVRIG antibody comprises the CH1-hinge-CH2-CH3 region from IgG1, IgG2, IgG3, or IgG4, wherein said hinge region optionally comprises mutations.

In some embodiments of the method of treatment said heavy chain variable domain is from the heavy chain of CHA.7.518.1.H4(S241P) (SEQ ID NO:4) and said light chain variable domain is from the light chain of CHA.7.518.1.H4(S241P) (SEQ ID NO:9).

In some embodiments of the method of treatment said anti-PVRIG antibody comprises a CL region of human kappa 2 light chain.

In some embodiments of the method of treatment, said pharmaceutical formulation comprises from 10 mM to 80 mM histidine, from 15 mM to 70 mM histidine, from 20 mM to 60 mM histidine, from 20 mM to 50 mM histidine, or from 20 mM to 30 mM histidine.

In some embodiments of the method of treatment, said pharmaceutical formulation comprises about 25 mM histidine.

In some embodiments of the method of treatment, said pharmaceutical formulation comprises from 30 mM to 100 mM NaCl, from 30 mM to 90 mM NaCl, from 40 mM to 80 mM NaCl, from 30 mM to 70 mM histidine, or from 45 mM to 70 mM NaCl.

In some embodiments of the method of treatment, said pharmaceutical formulation comprises about 60 mM NaCl.

In some embodiments of the method of treatment, said pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine.

In some embodiments of the method of treatment, said pharmaceutical formulation comprises about 100 mM L-arginine.

In some embodiments of the method of treatment, said pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, or from 0.009% to 0.05% w/v polysorbate 80.

In some embodiments of the method of treatment, said pharmaceutical formulation comprises about 0.01% polysorbate 80.

In some embodiments of the method of treatment, said pH is from 6 to 7.0.

In some embodiments of the method of treatment, said pH is from 6.3 to 6.8.

In some embodiments of the method of treatment, said pH is 6.5+/−0.2.

In some embodiments of the method of treatment, said anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL.

In some embodiments of the method of treatment, said formulation is stable at 2° C. to 8° C. for at least 1 week, 2 weeks, 3 weeks, 4 weeks, 5 weeks, 6 weeks, 7 weeks, 8 weeks, 9 weeks, or 10 weeks.

In some embodiments of the stable liquid pharmaceutical formulation, said formulation is stable at about 20° C. to 25° C. for at least 1 week, 2 weeks, 3 weeks, 4 weeks, 5 weeks, or 6 weeks.

In some embodiments of the method of treatment, said formulation is stable at 35° C. to 40° C. for at least 1 week, 2 weeks, 3 weeks, 4 weeks, or 5 weeks.

In some embodiments of the method of treatment, said anti-PVRIG antibody is at a concentration of about 20 mg/mL.

In some embodiments of the method of treatment, said anti-PVRIG antibody formulation comprises:

a) a heavy chain comprising:
i) a VH-CH1-hinge-CH2-CH3, wherein the VH is from CHA.7.518.1.H4(S241P) (SEQ ID NO:4) and wherein the CH1-hinge-CH2-CH3 region is from IgG4; and

b) a light chain comprising:
i) a VL-CL, wherein the VL from CHA.7.518.1.H4(S241P) (SEQ ID NO:9) and wherein the CL region is from human kappa 2 light chain.

In some embodiments of the method of treatment, said hinge region optionally comprises mutations.

In some embodiments of the method of treatment, said hinge region optionally comprises mutations.

In some embodiments of the method of treatment, said anti-PVRIG antibody formulation comprises:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13).

In some embodiments of the method of treatment, said anti-PVRIG antibody formulation comprises:

(a) an anti-PVRIG antibody, wherein said anti-PVRIG antibody comprises:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments of the method of treatment, said anti-PVRIG antibody formulation comprises:

(a) an anti-PVRIG antibody, wherein said anti-PVRIG antibody comprises:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments of the method of treatment, said formulation is administered at a dosage of about 0.01 mg/kg to about 20 mg/kg of the anti-PVRIG antibody. In some embodiments of the stable liquid pharmaceutical formulation, said formulation is administered at a dosage of about 0.01 mg/kg to about 10 mg/kg of the anti-PVRIG antibody.

In some embodiments of the method of treatment, said formulation is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg of the anti-PVRIG antibody.

In some embodiments of the method of treatment, said nivolumab is administered at a dosage of about 360 mg of nivoluman or 480 mg nivolumab.

In some embodiments of the method of treatment, said formulation is administered 20 mg/kg IV every 4 weeks.

In some embodiments of the method of treatment, said formulation is administered 20 mg/kg IV every for 4 weeks for for up to 24 months until disease progression, unacceptable toxicity, initiation of a new anticancer therapy, withdrawal of subject consent or death. In some embodiments, administration is up to 6 months, 12, months, 18 months or 24 months, until disease progression, unacceptable toxicity, initiation of a new anticancer therapy, withdrawal of subject consent or death.

In some embodiments of the method of treatment, said cancer selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, NSCLC, adenoid cystic cancer (including adenoid cystic carcinoma), primary peritoneal cancer, microsatellite stable primary peritoneal cancer, platinum resistant microsatellite stable primary peritoneal cancer, and/or Myelodysplastic syndromes (MDS).

The present invention provides for the use of nivolumab and an anti-PVRIG antibody in a method of treating cancer, wherein said anti-PVRIG antibody is administered as a stable liquid pharmaceutical formulation and, wherein said stable liquid pharmaceutical formulation of the anti-PVRIG antibody comprises:

(a) an anti-PVRIG antibody, wherein said anti-PVRIG antibody comprises:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments of the use in a method for treatment according to paragraph [0047], said anti-PVRIG antibody comprises a CH1-hinge-CH2-CH3 sequence of IgG4 (SEQ ID NO:17 or SEQ ID NO:50), wherein said hinge region optionally comprises mutations.

In some embodiments of the use in a method for treatment according to paragraphs [0047]-[0048], said anti-PVRIG antibody comprises the CH1-hinge-CH2-CH3 region from IgG1, IgG2, IgG3, or IgG4, wherein said hinge region optionally comprises mutations.

In some embodiments of the use in a method for treatment according to paragraphs [0047]-[0049], said heavy chain variable domain is from the heavy chain of CHA.7.518.1.H4(S241P) (SEQ ID NO:4) and said light chain variable domain is from the light chain of CHA.7.518.1.H4(S241P) (SEQ ID NO:9).

In some embodiments of the use in a method for treatment according to paragraphs [0047]-[0050], said anti-PVRIG antibody comprises a CL region of human kappa 2 light chain.

In some embodiments of the use in a method for treatment according to paragraphs [0047]-[0051], said pharmaceutical formulation comprises from 10 mM to 80 mM histidine, from 15 mM to 70 mM histidine, from 20 mM to 60 mM histidine, from 20 mM to 50 mM histidine, or from 20 mM to 30 mM histidine.

In some embodiments of the use in a method for treatment according to paragraphs [0047]-[0052], said pharmaceutical formulation comprises about 25 mM histidine.

In some embodiments of the use in a method for treatment according to paragraphs [0047]-[0053], said pharmaceutical formulation comprises from 30 mM to 100 mM NaCl, from 30 mM to 90 mM NaCl, from 40 mM to 80 mM NaCl, from 30 mM to 70 mM histidine, or from 45 mM to 70 mM NaCl.

In some embodiments of the use in a method for treatment according to paragraphs [0047]-[0054], said pharmaceutical formulation comprises about 60 mM NaCl.

In some embodiments of the use in a method for treatment according to paragraphs [0047]-[0055], said pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine.

In some embodiments of the use in a method for treatment according to paragraphs [0047]-[0056], said pharmaceutical formulation comprises about 100 mM L-arginine.

In some embodiments of the use in a method for treatment according to paragraphs [0047]-[0057], said pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, or from 0.009% to 0.05% w/v polysorbate 80.

In some embodiments of the use in a method for treatment according to paragraphs [0047]-[0058], said pharmaceutical formulation comprises about 0.01% polysorbate 80.

In some embodiments of the use in a method for treatment according to paragraphs [0047]-[0059], said pH is from 6 to 7.0.

In some embodiments of the use in a method for treatment according to paragraphs [0047]-[0060], said pH is from 6.3 to 6.8.

In some embodiments of the use in a method for treatment according to paragraphs [0047]-[0061], said pH is 6.5+/−0.2.

In some embodiments of the use in a method for treatment according to paragraphs [0047]-[0062], said anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL.

In some embodiments of the use in a method for treatment according to paragraphs [0047]-[063], said formulation is stable at 2° C. to 8° C. for at least 1 week, 2 weeks, 3 weeks, 4 weeks, 5 weeks, 6 weeks, 7 weeks, 8 weeks, 9 weeks, or 10 weeks.

In some embodiments of the use in a method for treatment according to paragraphs [0047]-[0064], said formulation is stable at about 20° C. to 25° C. for at least 1 week, 2 weeks, 3 weeks, 4 weeks, 5 weeks, or 6 weeks.

In some embodiments of the use in a method for treatment according to paragraphs [0047]-[0065], said formulation is stable at 35° C. to 40° C. for at least 1 week, 2 weeks, 3 weeks, 4 weeks, or 5 weeks.

In some embodiments of the use in a method for treatment according to paragraphs [0047]-[0066], said anti-PVRIG antibody is at a concentration of about 20 mg/mL.

In some embodiments of the use in a method for treatment according to paragraphs [0047]-[0067], said anti-PVRIG antibody formulation comprises:

a) a heavy chain comprising:
i) a VH-CH1-hinge-CH2-CH3, wherein the VH is from CHA.7.518.1.H4(S241P) (SEQ ID NO:4) and wherein the CH1-hinge-CH2-CH3 region is from IgG4; and

b) a light chain comprising:
i) a VL-CL, wherein the VL from CHA.7.518.1.H4(S241P) (SEQ ID NO:9) and wherein the CL region is from human kappa 2 light chain.

In some embodiments of the use in a method for treatment according to paragraphs [0047]-[0068], said hinge region optionally comprises mutations.

In some embodiments of the use in a method for treatment according to paragraphs [0047]-[0069], said hinge region optionally comprises mutations.

In some embodiments of the use in a method for treatment according to paragraphs [0047]-[0070], said anti-PVRIG antibody formulation comprises:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13).

In some embodiments of the use in a method for treatment according to paragraphs [0047]-[0071], said anti-PVRIG antibody formulation comprises:

(a) an anti-PVRIG antibody, wherein said anti-PVRIG antibody comprises:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments of the use in a method for treatment according to paragraphs [0047]-[0072], said anti-PVRIG antibody formulation comprises:

(a) an anti-PVRIG antibody, wherein said anti-PVRIG antibody comprises:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments of the use in a method for treatment according to paragraphs [0047]-[0073], said formulation is administered at a dosage of about 0.01 mg/kg to about 20 mg/kg of the anti-PVRIG antibody. In some embodiments of the stable liquid pharmaceutical formulation, said formulation is administered at a dosage of about 0.01 mg/kg to about 10 mg/kg of the anti-PVRIG antibody.

In some embodiments of the use in a method for treatment according to paragraphs [0047]-[0074], said formulation is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg of the anti-PVRIG antibody.

In some embodiments of the use in a method for treatment according to paragraphs [0047]-[0075], said nivolumab is administered at a dosage of about 360 mg of nivoluman or 480 mg nivolumab.

In some embodiments of the use in a method for treatment according to paragraphs [0047]-[0076], said formulation is administered 20 mg/kg every 4 weeks. In some embodiments of the use in a method for treatment according to paragraphs [0047]-[0076], said formulation is administered 20 mg/kg IVevery 4 weeks.

In some embodiments of the method of treatment according to paragraphs [0047]-[0077], said formulation is administered 20 mg/kg IV every for 4 weeks for for up to 24 months until disease progression, unacceptable toxicity, initiation of a new anticancer therapy, withdrawal of subject consent or death. In some embodiments, administration is up to 6 months, 12, months, 18 months or 24 months, until disease progression, unacceptable toxicity, initiation of a new anticancer therapy, withdrawal of subject consent and/or death.

In some embodiments of the use in a method for treatment according to paragraphd/d/s [0047]-[0078], said cancer selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, NSCLC, adenoid cystic cancer (including adenoid cystic carcinoma), primary peritoneal cancer, microsatellite stable primary peritoneal cancer, platinum resistant microsatellite stable primary peritoneal cancer, and/or Myelodysplastic syndromes (MDS).

### BRIEF DESCRIPTION OF THE DRAWINGS

FIG. 1 depicts the full-length sequence of human PVRIG.

FIG. 2 depicts the sequence of the human Poliovirus receptor-related 2 protein (PVLR2, also known as nectin-2, CD112 or herpesvirus entry mediator B, (HVEB)), the binding partner of PVRIG. PVLR2 is a human plasma membrane glycoprotein.

FIG. 3 depicts the variable heavy and light chains as well as the vhCDR1, vhCDR2, vhCDR3, vlCDR1, vlCDR2 and vlCDR3 sequences the CHA.7.518.1.H4(S241P) of the invention.

FIG. 4 depicts the sequences of human IgG1, IgG2, IgG3 and IgG4.

FIGS. 5A-5D depicts the sequences of other PVRIG antibodies that can be formulated according to stable liquid formulations of an anti-PVRIG antibody of the present invention.

FIG. 6 provides data showing the receptor occupancy at various dosages of CHA.7.518.1.H4(S241P) (heavy chain: SEQ ID NO:8; light chain: SEQ ID NO:13).

FIG. 7 provides data showing the receptor occupancy at various dosages of CHA.7.518.1.H4(S241P) (heavy chain: SEQ ID NO:8; light chain: SEQ ID NO:13).

FIG. 8 provides data showing PVRIG is a novel checkpoint in the TIGIT/DNAM-1 AXIS.

FIG. 9 provides data showing PVRIG inhibition reduces tumor growth in mouse cancer models.

FIG. 10 provides schematics of the study design.

FIG. 11 provides information regarding patient baseline characteristics.

FIG. 12 provides information regarding patient treatment disposition.

FIG. 13 provides information regarding treatment emergent adverse events.

FIG. 14 provides information regarding treatment emergent serious adverse events.

FIG. 15 provides a Swimmer's plot of the patient data.

FIG. 16 provides a Waterfall plot of the patient data.

FIG. 17 provides information regarding patients with stable disease and the dose-response relationship.

FIG. 18 provides best on treatment timepoint response information for patients with treatment refractory disease.

FIG. 19 provides a graph of treatment dosage data.

FIG. 20 provides a data regarding PVRIG engagement by anti-PVRIG using a receptor occupancy assay.

FIG. 21 provides patient baseline characteristics data.

FIG. 22 provides patient disposition summary.

FIG. 23 shows the dose escalation schema.

FIG. 24 provides the summary of adverse events—safety analysis set.

FIG. 25 provides the summary of serious adverse events leading to study treatment discontinuation (Arm A).

FIG. 26 provides the incidence of treatment emergent adverse events (TEAE) in ≥3 patients—monotherapy.

FIG. 27 provides the incidence of TEAEs in ≥3 patients—combination therapy.

FIG. 28 provides the incidence of serious TEAEs in all patients—monotherapy (n=18).

FIG. 29 provides the incidence of serious TEAEs in all patients—combination (n=13).

FIG. 30 provides the CHA.7.518.1.H4(S241P) PK profile following iv infusion at cycle 1 day 1—Arms A and B.

FIG. 31 provides the summary of investigator-assessed response (per recist v1.1 dlt-evaluable population) for Arms A and B.

FIG. 32A-32C provide Swimmer Plots of data from Arms A and B. Summary plot is provided in FIG. 32C.

FIG. 33 provides a Waterfall Plot of data from Arms A and B.

FIG. 34 provides data regarding CHA.7.518.1.H4(S241P)+nivolumab—confirmed PR in a patient with MSS (microsatellite stable status) colorectal cancer (ongoing study treatment 44 wks).

FIG. 35 provides data regarding CHA.7.518.1.H4(S241P) monotherapy—confirmed PR in a patient with MSS (microsatellite stable status) platinum resistant primary peritoneal cancer ongoing study treatment 25 wks.

### DETAILED DESCRIPTION OF THE INVENTION

### I. Introduction

Cancer can be considered as an inability of the patient to recognize and eliminate cancerous cells. In many instances, these transformed (e.g., cancerous) cells counteract immunosurveillance. There are natural control mechanisms that limit T-cell activation in the body to prevent unrestrained T-cell activity, which can be exploited by cancerous cells to evade or suppress the immune response. Restoring the capacity of immune effector cells—especially T cells—to recognize and eliminate cancer is the goal of immunotherapy. The field of immuno-oncology, sometimes referred to as “immunotherapy” is rapidly evolving, with several recent approvals of T cell checkpoint inhibitory antibodies such as Yervoy, Keytruda and Opdivo. These antibodies are generally referred to as “checkpoint inhibitors” because they block normally negative regulators of T cell immunity. It is generally understood that a variety of immunomodulatory signals, both costimulatory and coinhibitory, can be used to orchestrate an optimal antigen-specific immune response. Generally, these antibodies bind to checkpoint inhibitor proteins such as CTLA-4 and PD-1, which under normal circumstances prevent or suppress activation of cytotoxic T cells (CTLs). By inhibiting the checkpoint protein, for example through the use of antibodies that bind these proteins, an increased T cell response against tumors can be achieved. That is, these cancer checkpoint proteins suppress the immune response; when the proteins are blocked, for example using antibodies to the checkpoint protein, the immune system is activated, leading to immune stimulation, resulting in treatment of conditions such as cancer and infectious disease.

The present invention is directed to formulations comprising antibodies to human Poliovirus Receptor Related Immunoglobulin Domain Containing Protein, or “PVRIG”, sometimes also referred to herein as “PV protein”. PVRIG is expressed on the cell surface of NK and T-cells and shares several similarities to other known immune checkpoints.

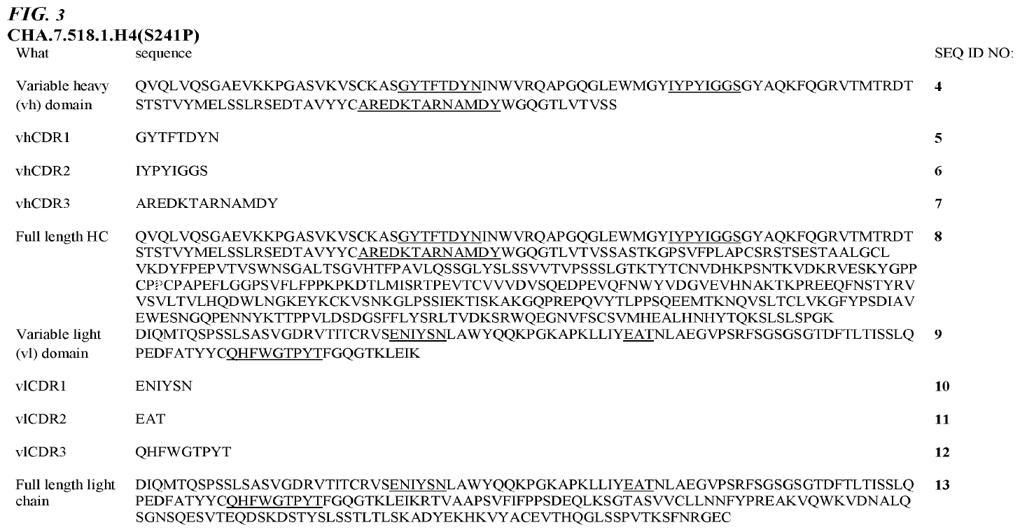

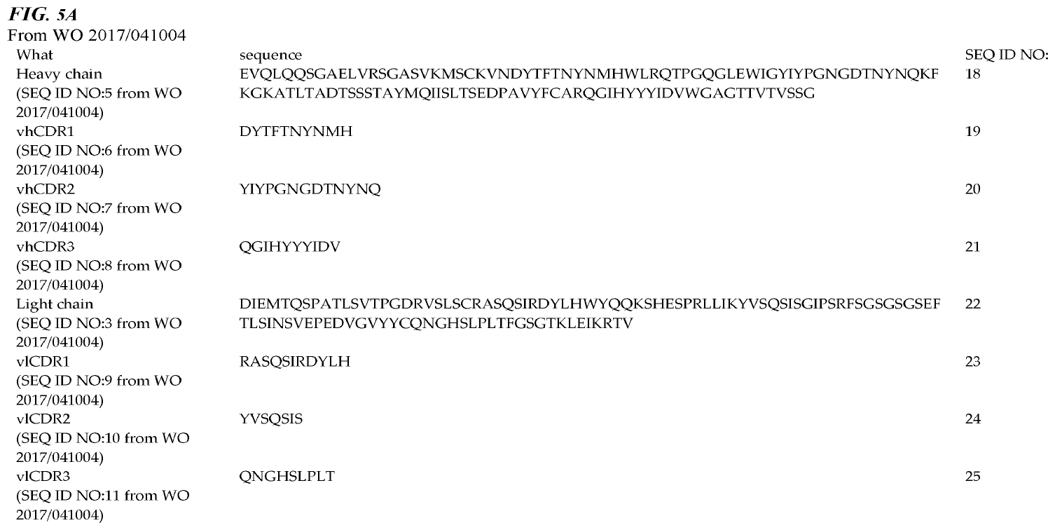

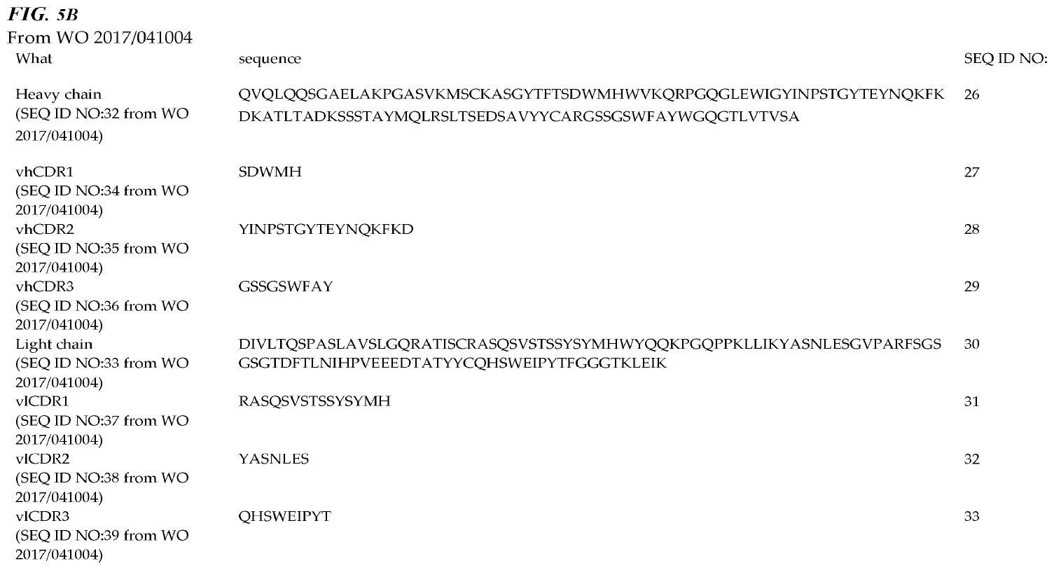

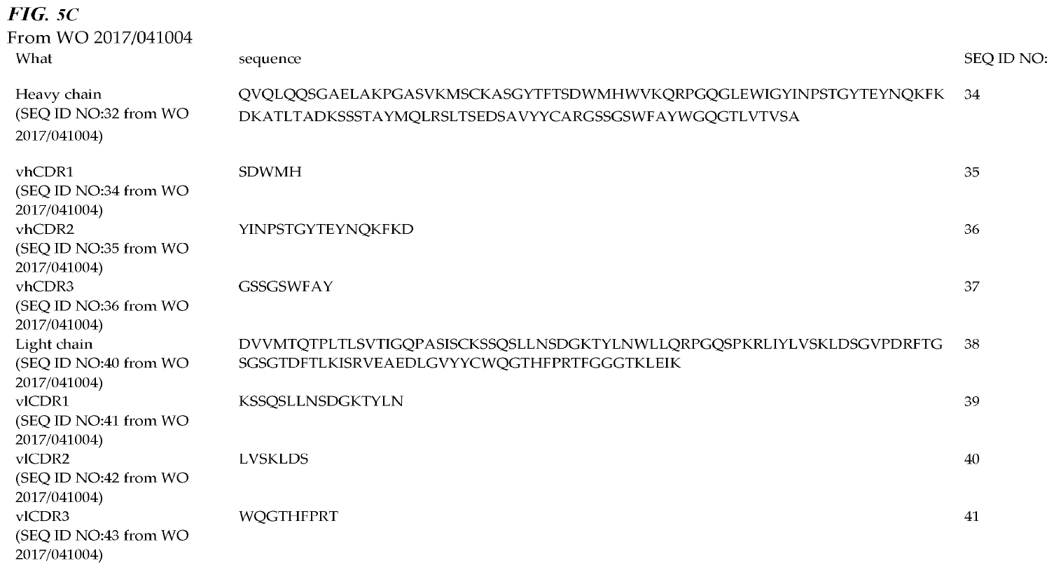

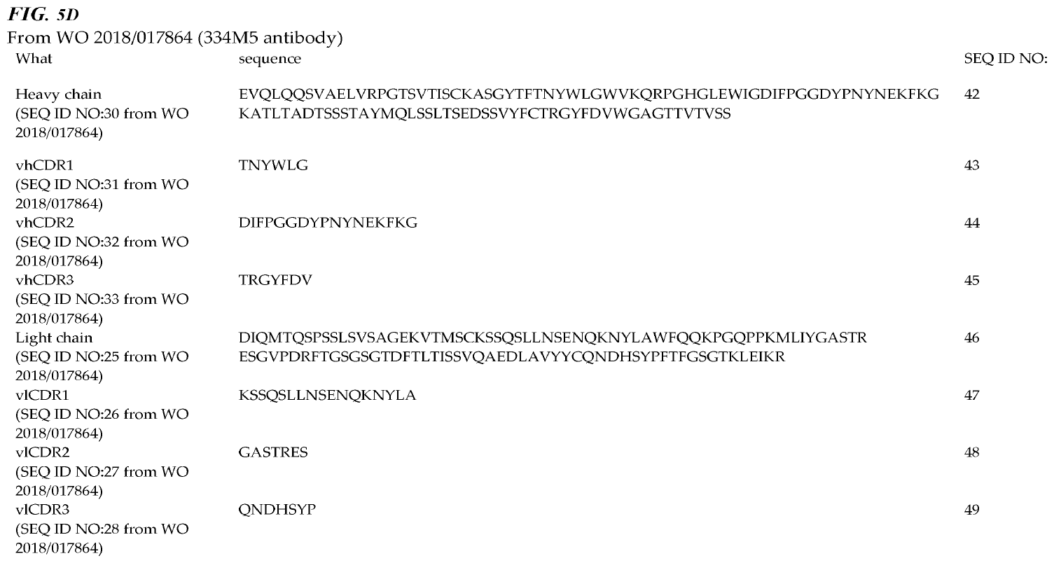

Accordingly, the present invention provides formulations comprising antibodies, including antigen binding domains, that bind to the human PVRIG and peptides thereof and methods of activating T cells and/or NK cells to treat diseases such as cancer and infectious diseases, and other conditions where increased immune activity results in treatment. In particular, the invention provides formulations comprising antibodies comprising heavy and light chains as well as the vhCDR1, vhCDR2, vhCDR3, vlCDR1, vlCDR2 and vlCDR3 sequences from CHA.7.518.1.H4(S241P). In some embodiments, anti-PVRIG antibodies include those with CDRs identical to those shown in FIG. 3. In some embodiments, anti-PVRIG antibodies include those with CDRs identical to those shown in FIGS. 5A-5D, as well as anti-PVRIG antibodies comprising the heavy and light chains as provided in FIGS. 5A-5D.

### ii. PVRIG Proteins

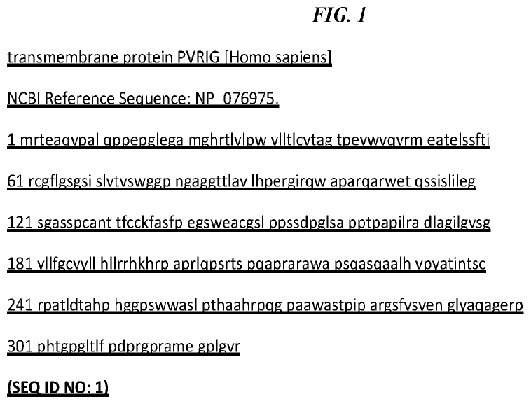

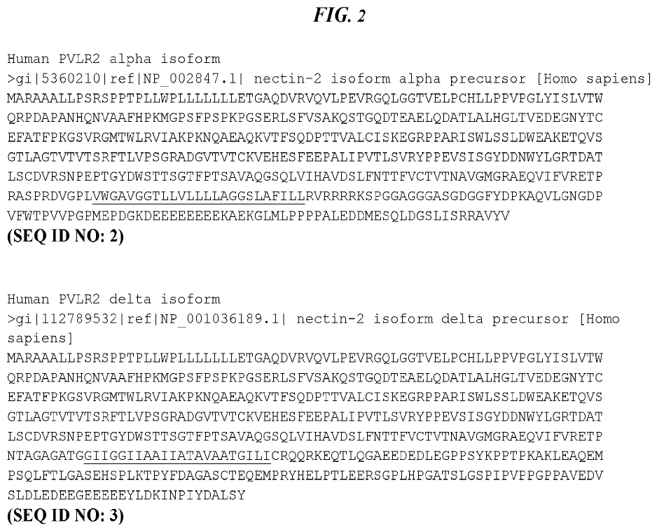

The present invention provides formulations comprising antibodies that specifically bind to PVRIG proteins. “Protein” in this context is used interchangeably with “polypeptide”, and includes peptides as well. The present invention provides antibodies that specifically bind to PVRIG proteins. PVRIG is a transmembrane domain protein of 326 amino acids in length, with a signal peptide (spanning from amino acid 1 to 40), an extracellular domain (spanning from amino acid 41 to 171), a transmembrane domain (spanning from amino acid 172 to 190) and a cytoplasmic domain (spanning from amino acid 191 to 326). The full length human PVRIG protein is shown in FIG. 1. There are two methionines that can be start codons, but the mature proteins are identical.

Accordingly, as used herein, the term “PVRIG” or “PVRIG protein” or “PVRIG polypeptide” may optionally include any such protein, or variants, conjugates, or fragments thereof, including but not limited to known or wild type PVRIG, as described herein, as well as any naturally occurring splice variants, amino acid variants or isoforms, and in particular the ECD fragment of PVRIG. The term “soluble” form of PVRIG is also used interchangeably with the terms “soluble ectodomain (ECD)” or “ectodomain” or “extracellular domain (ECD) as well as “fragments of PVRIG polypeptides”, which may refer broadly to one or more of the following optional polypeptides:

The PVRIG proteins contain an immunoglobulin (Ig) domain within the extracellular domain, which is a PVR-like Ig fold domain. The PVR-like Ig fold domain may be responsible for functional counterpart binding, by analogy to the other B7 family members. The PVR-like Ig fold domain of the extracellular domain includes one disulfide bond formed between intra domain cysteine residues, as is typical for this fold and may be important for structure-function. These cysteines are located at residues 22 and 93 (or 94). In one embodiment, there is provided a soluble fragment of PVRIG that can be used in testing of PVRIG antibodies. Included within the definition of PVRIG proteins are PVRIG ECD fragments, including know ECD fragments such as those described in U.S. Pat. No. 9,714,289, incorporate by reference herein in its entirety for all purposes.

As noted herein and more fully described below, the anti-PVRIG antibodies (including antigen-binding fragments) that both bind to PVRIG and prevent activation by PVRL2 (e.g. most commonly by blocking the interaction of PVRIG and PVLR2), are used to enhance T cell and/or NK cell activation and be used in treating diseases such as cancer and pathogen infection.

### III. Antibodies

Accordingly, the invention provides anti-PVRIG antibodies that can be formulated according to the formulations described herein and which are provided in FIG. 3 (e.g., including anti-PVRIG antibodies including those with CDRs identical to those shown in FIG. 3). PVRIG, also called Poliovirus Receptor Related Immunoglobulin Domain Containing Protein, Q6DKI7 or C7orf15, relates to amino acid and nucleic acid sequences shown in RefSeq accession identifier NP_076975, shown in FIG. 1. The antibodies of the invention are specific for the PVRIG extracellular domain.

As is discussed below, the term “antibody” is used generally. Antibodies that find use in the present invention can take on a number of formats as described herein, including traditional antibodies as well as antibody derivatives, fragments and mimetics, described below. In general, the term “antibody” includes any polypeptide that includes at least one antigen binding domain, as more fully described below. Antibodies may be polyclonal, monoclonal, xenogeneic, allogeneic, syngeneic, or modified forms thereof, as described herein, with monoclonal antibodies finding particular use in many embodiments. In some embodiments, antibodies of the invention bind specifically or substantially specifically to PVRIG molecules. The terms “monoclonal antibodies” and “monoclonal antibody composition”, as used herein, refer to a population of antibody molecules that contain only one species of an antigen-binding site capable of immunoreacting with a particular epitope of an antigen, whereas the term “polyclonal antibodies” and “polyclonal antibody composition” refer to a population of antibody molecules that contain multiple species of antigen-binding sites capable of interacting with a particular antigen. A monoclonal antibody composition, typically displays a single binding affinity for a particular antigen with which it immunoreacts.

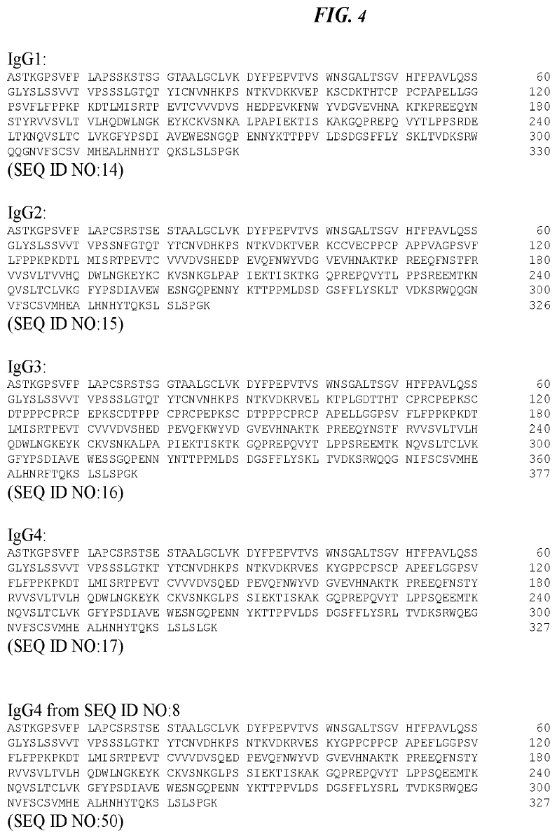

Traditional full length antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to the IgG class, which has several subclasses, including, but not limited to IgG1, IgG2, IgG3, and IgG4. Thus, “isotype” as used herein is meant any of the subclasses of immunoglobulins defined by the chemical and antigenic characteristics of their constant regions. While the exemplary antibodies herein designated “CPA” are based on IgG1 heavy constant regions, as shown in FIG. 4, the anti-PVRIG antibodies of the invention include those using IgG2, IgG3 and IgG4 sequences, or combinations thereof. For example, as is known in the art, different IgG isotypes have different effector functions which may or may not be desirable. Accordingly, the CPA antibodies of the invention can also swap out the IgG1 constant domains for IgG2, IgG3 or IgG4 constant domains (depicted in FIG. 4), with IgG2 and IgG4 finding particular use in a number of situations, for example for ease of manufacture or when reduced effector function is desired, the latter being desired in some situations.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions”.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

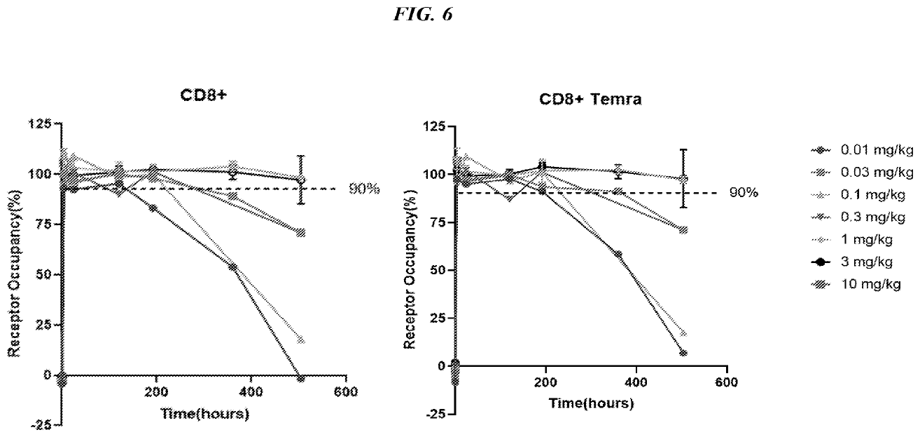

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region, although sometimes the numbering is shifted slightly as will be appreciated by those in the art; Kabat et al., SEQUENCES OF PROTEINS OF IMMUNOLOGICAL INTEREST, 5 th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below and shown in FIG. 6A-6D.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5 th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Accordingly, the invention provides variable heavy domains, variable light domains, heavy constant domains, light constant domains and Fc domains to be used as outlined herein. By “variable region” as used herein is meant the region of an immunoglobulin that comprises one or more Ig domains substantially encoded by any of the Vκ or Vλ, and/or VH genes that make up the kappa, lambda, and heavy chain immunoglobulin genetic loci respectively. Accordingly, the variable heavy domain comprises vhFR1-vhCDR1-vhFR2-vhCDR2-vhFR3-vhCDR3-vhFR4, and the variable light domain comprises v1FR1-v1CDR1-v1FR2-v1CDR2-v1FR3-v1CDR3-v1FR4. By “heavy constant region” herein is meant the CH1-hinge-CH2-CH3 portion of an antibody. By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

Thus, “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody. As will be appreciated by those in the art, these generally are made up of two chains.

Throughout the present specification, either the IMTG numbering system or the Kabat numbering system is generally used when referring to a residue in the variable domain (approximately, residues 1-107 of the light chain variable region and residues 1-113 of the heavy chain variable region) (e.g, Kabat et al., supra (1991)). EU numbering as in Kabat is generally used for constant domains and/or the Fc domains.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning”. Specific bins are described below.

Included within the definition of “antibody” is an “antigen-binding portion” of an antibody (also used interchangeably with “antigen-binding fragment”, “antibody fragment” and “antibody derivative”). That is, for the purposes of the invention, an antibody of the invention has a minimum functional requirement that it bind to a PVRIG antigen. As will be appreciated by those in the art, there are a large number of antigen fragments and derivatives that retain the ability to bind an antigen and yet have alternative structures, including, but not limited to, (i) the Fab fragment consisting of VL, VH, CL and CH1 domains, (ii) the Fd fragment consisting of the VH and CH1 domains, (iii) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (iv) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference), (v) “domain antibodies” or “dAb” (sometimes referred to as an “immunoglobulin single variable domain”, including single antibody variable domains from other species such as rodent (for example, as disclosed in WO 00/29004), nurse shark and Camelid V-HH dAbs, (vi) SMIPs (small molecule immunopharmaceuticals), camelbodies, nanobodies and IgNAR.

Still further, an antibody or antigen-binding portion thereof (antigen-binding fragment, antibody fragment, antibody portion) may be part of a larger immunoadhesion molecules (sometimes also referred to as “fusion proteins”), formed by covalent or noncovalent association of the antibody or antibody portion with one or more other proteins or peptides. Examples of immunoadhesion molecules include use of the streptavidin core region to make a tetrameric scFv molecule and use of a cysteine residue, a marker peptide and a C-terminal polyhistidine tag to make bivalent and biotinylated scFv molecules. Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

In general, the anti-PVRIG antibodies of the invention are recombinant. “Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “recombinant antibody”, as used herein, includes all antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

A. Optional Antibody Engineering

The anti-PVRIG antibodies (e.g., anti-PVRIG antibodies including those with CDRs identical to those shown in FIG. 3) of the invention can be modified, or engineered, to alter the amino acid sequences by amino acid substitutions.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

As discussed herein, amino acid substitutions can be made to alter the affinity of the CDRs for the PVRIG protein (including both increasing and decreasing binding, as is more fully outlined below), as well as to alter additional functional properties of the antibodies. For example, the antibodies may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of C_{H1} is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In some embodiments, amino acid substitutions can be made in the Fc region, in general for altering binding to FcγR receptors. By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII-1 (CD16), and FcγRIII-2 (CD16-2), as well as any undiscovered mouse FcγRs or FcγR isoforms or allotypes.

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell. Similarly, decreased binding to FcγRIIb (an inhibitory receptor) can be beneficial as well in some circumstances. Amino acid substitutions that find use in the present invention include those listed in U.S. Ser. Nos. 11/124,620 (particularly FIG. 41) and U.S. Pat. No. 6,737,056, both of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 299T and 297N.

In addition, the antibodies of the invention are modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the Cm or CL region to contain a salvage receptor binding epitope taken from two loops of a CH2 domain of an Fc region of an IgG, as described in U.S. Pat. Nos. 5,869,046 and 6,121,022 by Presta et al. Additional mutations to increase serum half-life are disclosed in U.S. Pat. Nos. 8,883,973, 6,737,056 and 7,371,826, and include 428L, 434A, 434S, and 428L/434S.

In yet other embodiments, the Fc region is altered by replacing at least one amino acid residue with a different amino acid residue to alter the effector functions of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 are shown to improve binding to FcγRIII. Additionally, the following combination mutants are shown to improve FcγRIII binding: T256A/S298A, S298A/E333A, S298A/K224A and S298A/E333A/K334A. Furthermore, mutations such as M252Y/S254T/T256E or M428L/N434S improve binding to FcRn and increase antibody circulation half-life (see Chan C A and Carter P J (2010) Nature Rev Immunol 10:301-316).

In still another embodiment, the antibody can be modified to abrogate in vivo Fab arm exchange. Specifically, this process involves the exchange of IgG4 half-molecules (one heavy chain plus one light chain) between other IgG4 antibodies that effectively results in bispecific antibodies which are functionally monovalent. Mutations to the hinge region and constant domains of the heavy chain can abrogate this exchange (see Aalberse, R C, Schuurman J., 2002, Immunology 105:9-19).

In still another embodiment, the glycosylation of an antibody is modified. For example, an aglycosylated antibody can be made (i.e., the antibody lacks glycosylation). Glycosylation can be altered to, for example, increase the affinity of the antibody for antigen or reduce effector function such as ADCC. Such carbohydrate modifications can be accomplished by, for example, altering one or more sites of glycosylation within the antibody sequence, for example N297. For example, one or more amino acid substitutions can be made that result in elimination of one or more variable region framework glycosylation sites to thereby eliminate glycosylation at that site.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. For example, the cell lines Ms704, Ms705, and Ms709 lack the fucosyltransferase gene, FUT8 (α(1,6) fucosyltransferase), such that antibodies expressed in the Ms704, Ms705, and Ms709 cell lines lack fucose on their carbohydrates. The Ms704, Ms705, and Ms709 FUT8 cell lines are created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the α 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase α-L-fucosidase removes fucosyl residues from antibodies (Tarentino, A. L. et al. (1975) Biochem. 14:5516-23).

Another modification of the antibodies herein that is contemplated by the invention is pegylation or the addition of other water soluble moieties, typically polymers, e.g., in order to enhance half-life. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies according to at least some embodiments of the invention. See for example, EP 0 154 316 by Nishimura et al. and EP 0 401 384 by Ishikawa et al.

In addition to substitutions made to alter binding affinity to FcγRs and/or FcRn and/or increase in vivo serum half-life, additional antibody modifications can be made, as described in further detail below.

In some cases, affinity maturation is done. Amino acid modifications in the CDRs are sometimes referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

In some embodiments, one or more amino acid modifications are made in one or more of the CDRs of the PVRIG antibodies of the invention. In general, only 1 or 2 or 3-amino acids are substituted in any single CDR, and generally no more than from 1, 2, 3. 4, 5, 6, 7, 8 9 or 10 changes are made within a set of CDRs. However, it should be appreciated that any combination of no substitutions, 1, 2 or 3 substitutions in any CDR can be independently and optionally combined with any other substitution.

Affinity maturation can be done to increase the binding affinity of the antibody for the PVRIG antigen by at least about 10% to 50-100-150% or more, or from 1 to 5 fold as compared to the “parent” antibody. Preferred affinity matured antibodies will have nanomolar or even picomolar affinities for the PVRIG antigen. Affinity matured antibodies are produced by known procedures. See, for example, Marks et al., 1992, Biotechnology 10:779-783 that describes affinity maturation by variable heavy chain (VH) and variable light chain (VL) domain shuffling. Random mutagenesis of CDR and/or framework residues is described in: Barbas, et al. 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154(7):3310-9; and Hawkins et al, 1992, J. Mol. Biol. 226:889-896, for example.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of the enumerated antibodies of the invention. In addition, as outlined below, amino acid modifications can also independently and optionally be made in any region outside the CDRs, including framework and constant regions.

### IV. PVRIG Antibodies

The present invention provides anti-PVRIG antibodies. (For convenience, “anti-PVRIG antibodies” and “PVRIG antibodies” are used interchangeably). The anti-PVRIG antibodies of the invention specifically bind to human PVRIG, and preferably the ECD of human PVRIG1, as depicted in FIG. 3, including, e.g., anti-PVRIG antibodies including those with CDRs identical to those shown in FIG. 3.

Specific binding for PVRIG or a PVRIG epitope can be exhibited, for example, by an antibody having a KD of at least about 10⁻⁴ M, at least about 10⁻⁵ M, at least about 10⁻⁶ M, at least about 10⁻⁷ M, at least about 10⁻⁸M, at least about 10⁻⁹M, alternatively at least about 10⁻¹⁰ M at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the PVRIG antigen or epitope.

However, as shown in the Examples, for optimal binding to PVRIG expressed on the surface of NK and T-cells, the antibodies preferably have a KD less 50 nM and most preferably less than 1 nM, with less than 0.1 nM and less than 1 pM and 0.1 pM finding use in the methods of the invention.

Also, specific binding for a particular antigen or an epitope can be exhibited, for example, by an antibody having a KA or Ka for a PVRIG antigen or epitope of at least 20-, 50-, 100-, 500-, 1000-, 5,000-, 10,000- or more times greater for the epitope relative to a control, where KA or Ka refers to an association rate of a particular antibody-antigen interaction. s

In some embodiments, the anti-PVRIG antibodies of the invention bind to human PVRIG with a K_{D} of 100 nM or less, 50 nM or less, 10 nM or less, or 1 nM or less (that is, higher binding affinity), or 1 pM or less, wherein K_{D} is determined by known methods, e.g. surface plasmon resonance (SPR, e.g. Biacore assays), ELISA, KINEXA, and most typically SPR at 25° or 37° C.

The invention provides antigen binding domains, including full length antibodies, which contain a number of specific, enumerated sets of 6 CDRs, as provided in FIG. 3. The invention provides antigen binding domains, including full length antibodies, which contain a number of specific, enumerated sets of 6 CDRs, as provided in FIG. 3.

The invention further provides variable heavy and light domains as well as full length heavy and light chains.

As discussed herein, the invention further provides variants of the above components, including variants in the CDRs, as outlined above. In addition, variable heavy chains can be at least 80%, at least 90%, at least 95%, at least 98% or at least 99% identical to the “VH” sequences herein, and/or contain from 1, 2, 3, 4, 5, 6, 7, 8, 9, 10 amino acid changes, or more, when Fc variants are used. Variable light chains are provided that can be at least 80%, at least 90%, at least 95%, at least 98% or at least 99% identical to the “VL” sequences herein, and/or contain from 1, 2, 3, 4, 5, 6, 7, 8, 9, 10 amino acid changes, or more, when Fc variants are used. Similarly, heavy and light chains are provided that are at least 80%, at least 90%, at least 95%, at least 98% or at least 99% identical to the “HC” and “LC” sequences herein, and/or contain from 1, 2, 3, 4, 5, 6, 7, 8, 9, 10 amino acid changes, or more, when Fc variants are used.

Accordingly, the present invention provides antibodies, usually full length or scFv domains, that comprise the following CHA sets of CDRs, the sequences of which are shown in FIG. 3:

CHA.7.518.1.H4(S241P)vhCDR1, CHA.7.518.1.H4(S241P)vhCDR2, CHA.7.518.1.H4(S241P)vhCDR3, CHA.7.518.1.H4(S241P)v1CDR1, CHA.7.518.1.H4(S241P)v1CDR2, and CHA.7.518.1.H4(S241P)v1CDR3.

In addition, the framework regions of the variable heavy and variable light chains can be humanized as is known in the art (with occasional variants generated in the CDRs as needed), and thus humanized variants of the VH and VL chains of FIG. 3 can be generated. Furthermore, the humanized variable heavy and light domains can then be fused with human constant regions, such as the constant regions from IgG1, IgG2, IgG3 and IgG4.

In addition, also included are sequences that may have the identical CDRs but changes in the variable domain (or entire heavy or light chain). For example, PVRIG antibodies include those with CDRs identical to those shown in FIG. 3 or FIGS. 5A-5D but whose identity along the variable region can be lower, for example 95 or 98% percent identical. For example, PVRIG antibodies include those with CDRs identical to those shown in FIG. 3 but whose identity along the variable region can be lower, for example 95 or 98% percent identical, and in some embodiments at least 95% or at least 98%.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In general, the percentage identity for comparison between PVRIG antibodies is at least 75%, at least 80%, at least 90%, with at least about 95, 96, 97, 98 or 99% percent identity being preferred. The percentage identity may be along the whole amino acid sequence, for example the entire heavy or light chain or along a portion of the chains. For example, included within the definition of the anti-PVRIG antibodies of the invention are those that share identity along the entire variable region (for example, where the identity is 95 or 98% identical along the variable regions, and in some embodiments at least 95% or at least 98%), or along the entire constant region, or along just the Fc domain.

### V. Formulations of Anti-PVRIG Antibodies

The anti-PVRIG antibodies and/or antigen binding portions thereof compositions (e.g., anti-PVRIG antibodies including those with CDRs identical to those shown in FIG. 3) can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980). Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™, or polyethylene glycol (PEG).

In a preferred embodiment, the pharmaceutical composition that comprises anti-PVRIG antibodies including those with CDRs identical to those shown in FIG. 3) of the invention may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferrably sterile. This is readily accomplished by filtration through sterile filtration membranes or other methods.

As used herein, the term “activity” refers to a functional activity or activities of anti-PVRIG antibodies and/or antigen binding portions thereof. Functional activities include, but are not limited to, biological activity anor binding affinity.

As used herein, the term “stability” is used in a structural context, e.g., relating to the structural integrity of an anti-PVRIG antibody and/or antigen binding portion thereof, or in a functional context, e.g., relating to a an anti-PVRIG antibody and/or antigen binding portion thereof's ability to retain its function and/or activity over time (e.g., including anti-PVRIG antibody and/or antigen binding portion thereof stability or anti-PVRIG antibody and/or antigen binding portion thereof formulation stability, wherein the anti-PVRIG antibody includes those with CDRs identical to those shown in FIG. 3). As will be appreciated, the an anti-PVRIG antibody and/or antigen binding portion thereof under discussion may be contained within a formulation in accordance with the methods and compositions described herein, and the stability of that protein refers to its stability in that formulation. In some embodiments, the stability of an an anti-PVRIG antibody and/or antigen binding portion thereof composition is determined by measuring the binding activity of the composition, including for example, using the assays described in the application and figures provided herewith, as well as an other applicable assays known in the art. In some embodiments, the stability of an anti-PVRIG antibody and/or antigen binding portion thereof composition is formulated with sugar, sugar alcohol, and/or non-ionic surfactant, as described herein, is compared to an anti-PVRIG antibody and/or antigen binding portion thereof composition formulated without the at least one amino acid, salt, and/or non-ionic surfactant and/or with a different combination of components. In some embodiments, the formulation does not comprise a sugar and/or sugar alcohol.

As used herein, a “storage stable” aqueous an anti-PVRIG antibody and/or antigen binding portion thereof composition refers to a an anti-PVRIG antibody and/or antigen binding portion thereof comprising solution that has been formulated to increase the stability of the protein in solution, for example by at least 10%, over a given storage time. In the context of the present disclosure, an anti-PVRIG antibody and/or antigen binding portion thereof can be made “storage stable” by the addition of at least one amino acid, salt, or non-ionic surfactant as a stabilizing agent. In some embodiments, the stability of the an anti-PVRIG antibody and/or antigen binding portion thereof in any given formulation can be measured, for example, by monitoring the formation of aggregates, loss of bulk binding activity, or formation of degradation products, over a period of time. The absolute stability of a formulation, and the stabilizing effects of the sugar, sugar alcohol, or non-ionic surfactant, will vary dependent upon the particular composition being stabilized. In one embodiment, the stability of an anti-PVRIG antibody and/or antigen binding portion thereof composition is determined by measuring the anti-PVRIG antibody and/or antigen binding portion thereof binding activity of the composition. For example, by using using an ELISA or other binding activity assay. In one embodiment, the stability of an anti-PVRIG antibody and/or antigen binding portion thereof composition formulated with sugar, sugar alcohol, and/or non-ionic surfactant, as described herein, is compared to an anti-PVRIG antibody and/or antigen binding portion thereof composition formulated without the a least one amino acid, salt, and/or non-ionic surfactant and/or with a different combination of components. In some embodiments, the formulation does not comprise a sugar and/or sugar alcohol.

As used herein, “shelf-life” refers to the period of time a formulation maintains a predetermined level of stability at a predetermined temperature. In particular embodiments, the predetermined temperature refers to frozen (e.g., −80° C., −25° C., 0° C.), refrigerated (e.g., 0° to 10° C.), or room temperature (e.g., 18° C. to 32° C.) storage.

As used herein, the term “time of stability” refers to the length of time a formulation is considered stable. For example, the time of stability for a formulation may refer to the length of time for which the level of protein aggregation and/or degradation in the formulation remains below a certain threshold (e.g., 1%, 2%, 3%, 4%, 5%, 6%, 7%, 8%, 9%, 10%, 11%, 12%, 13%, 14%, 15%, 16%, 17%, 18%, 19%, 20%, etc.), and/or the length of time a formulation maintains biological activity above a certain threshold (e.g., 100%, 95%, 90%, 85%, 80%, 75%, 70%, 65%, 60%, 55%, 50%, etc.) of the amount of activity (including, for example, binding activity) present in the formulation at the start of the storage period.

In the context of the present disclosure, a storage stable aqueous composition of a an anti-PVRIG antibody and/or antigen binding portion thereof formulated with a sugar, sugar alcohol, and/or non-ionic surfactant will have a longer time of stability than a composition of the same an anti-PVRIG antibody and/or antigen binding portion thereof formulated without the at least one amino acid, salt, and/or non-ionic surfactant. In some embodiments, a storage stable aqueous composition of an anti-PVRIG antibody and/or antigen binding portion thereof, will have a time of stability that is, for example, at least 10% greater than the time of stability for the an anti-PVRIG antibody and/or antigen binding portion thereof composition formulated in the absence of the at least one amino acid, salt, and/or non-ionic surfactant, or at least 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190% greater, or at least 2 times greater, or at least 2.5 times, 3.0 times, 3.5 times, 4.0 times, 4.5 times, 5.0 times, 5.5 times, 6.0 times, 6.5 times, 7.0 times, 7.5 times, 8.0 times, 8.5 times, 9.0 times, 9.5 times, 10 times, or more times greater than the time of stability for the an anti-PVRIG antibody and/or antigen binding portion thereof composition formulated in the absence of the at least amino acid, salt, and/or non-ionic surfactant.

As used herein, “BDS” refers to “Bulk Drug Substance.”

A. Stabilized Liquid Formulations

In some embodiments, the present disclosure provides stabilized aqueous formulations of an anti-PVRIG antibody and/or antigen binding portion thereof (e.g., anti-PVRIG antibodies including those with CDRs identical to those shown in FIG. 3). The following embodiments are based in part on the discovery that inclusion of at least one amino acid, salt, and/or non-ionic surfactant stabilizes the liquid anti-PVRIG antibody and/or antigen binding portion thereof compositions, as compared to compositions lacking the at least one amino acid, salt, and/or non-ionic surfactant. In some embodiments, the formulation does not comprise a sugar and/or sugar alcohol.

As will be recognized by one of skill in the art, an anti-PVRIG antibody and/or antigen binding portion thereof formulated according to the embodiments provided herein may contain, in addition to the components explicitly disclosed, counter ions contributed by the inclusion of solution components or pH modifying agents, for example, sodium or potassium contributed from an acetate salt, sodium hydroxide, or potassium hydroxide or chloride contributed by calcium chloride or hydrochloric acid. In the context of the present disclosure, a storage stable an anti-PVRIG antibody and/or antigen binding portion thereof composition consisting of or consisting essentially of a given formulation may further comprise one or more counter ion, as necessitated by the formulation process at a particular pH.

In one embodiment, a storage stable anti-PVRIG antibody and/or antigen binding portion provided herein will be stabilized at refrigerated temperature (i.e., between 2° C. and 10° C.) for a period of time. For example, in one embodiment, a stable liquid pharmaceutical formulations comprising an anti-PVRIG antibody or antigen binding fragment thereof will be stable when stored at refrigerated temperature for at least 4 days. In other embodiments, the anti-PVRIG antibody and/or antigen binding portion composition will be stabile at refrigerated temperature for at least 1, 2, 3, 4, 5, 6, 7, 8, 9, 10, 11, 12, 13, 14, 21, 28, or more days. In some embodiments, the anti-PVRIG antibody and/or antigen binding portion composition will be stable for at least 1 week, 2 weeks, 3 weeks, 4 weeks, 5 weeks, 6 weeks, 7 weeks, 8 weeks, 9 weeks, 10 weeks, or more. In some embodiments, the anti-PVRIG antibody and/or antigen binding portion composition will be stable for at least 1 month. In some embodiments, the composition will be stable for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In some embodiments, the anti-PVRIG antibody and/or antigen binding portion composition will be stable for an extended period of time when stored at a temperature between 2° C. and 8° C.

In one embodiment, a stable liquid pharmaceutical formulations comprising an anti-PVRIG antibody or antigen binding fragment thereof provided herein will be stabilized at room temperature (i.e., between 18° C. and 32° C.) for a period of time. For example, in one embodiment, a stable liquid pharmaceutical formulations comprising an anti-PVRIG antibody or antigen binding fragment thereof will be stable when stored at room temperature for at least 4 days. In some embodiments, the anti-PVRIG antibody and/or antigen binding portion composition will be stabile at room temperature for at least 1, 2, 3, 4, 5, 6, 7, 8, 9, 10, 11, 12, 13, 14, 21, 28, or more days. In some embodiments, the anti-PVRIG antibody and/or antigen binding portion composition will be stable for at least 1 week, 2 weeks, 3 weeks, 4 weeks, 5 weeks, 6 weeks, or more. In some embodiments, the anti-PVRIG antibody and/or antigen binding portion composition will be stable for at least 1 month. In yet other embodiments, the composition will be stable for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In some embodiments, room temperature refers to between 20° C. and 30° C., between 21° C. and 29° C., between 22° C. and 28° C., between 23° C. and 27° C., between 24° C. and 26° C., or about 25° C. In some embodiments, the anti-PVRIG antibody and/or antigen binding portion composition will be stable for an extended period of time when stored at a temperature between 20° C. and 25° C. In some embodiments, the anti-PVRIG antibody and/or antigen binding portion composition will be stable for an extended period of time when stored at a temperature of about 25° C.

In one embodiment, a storage stable anti-PVRIG antibody and/or antigen binding portion provided herein will be stabilized at elevated temperature (i.e., between 32° C. and 42° C.) for a period of time. For example, in one embodiment, a stable liquid pharmaceutical formulations comprising an anti-PVRIG antibody or antigen binding fragment thereof will be stable when stored at elevated temperature for at least 4 days. In some embodiments, the anti-PVRIG antibody and/or antigen binding portion composition will be stabile at elevated temperature for at least 1, 2, 3, 4, 5, 6, 7, 8, 9, 10, 11, 12, 13, 14, 21, 28, or more days. In some embodiments, the anti-PVRIG antibody and/or antigen binding portion composition will be stable for at least 1 week, 2 weeks, 3 weeks, 4 weeks, 5 weeks, or more. In some embodiments, the anti-PVRIG antibody and/or antigen binding portion composition will be stable for at least 1 month. In yet other embodiments, the anti-PVRIG antibody and/or antigen binding portion composition will be stable for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In some embodiments, the anti-PVRIG antibody and/or antigen binding portion composition will be stable for an extended period of time when stored at a temperature between 35° C. and 40° C.

In one embodiment, a stored anti-PVRIG antibody and/or antigen binding portion composition is considered storage stable as long as the composition maintains at least 40% of the antibody binding activity present at the start of the storage period (e.g., at time=0). In another embodiment, a stored composition is considered stable as long as the composition maintains at least 45%, 50%, 55%, 60%, 65%, 70%, 75%, 80%, 85%, 90%, 95% or more of the antibody binding activity present at the start of the storage period (e.g., at time=0). In one embodiment, antibody binding activity is measure using any assay known in the art.

In some embodiments, an anti-PVRIG antibody and/or antigen binding portion composition is considered to have been stabilized by the addition of a stabilizing agent (e.g., at least one amino acid, salt, and/or non-ionic surfactant) when the anti-PVRIG antibody and/or antigen binding portion composition contains at least 10% more antibody binding activity after storage for a period of time, as compared to an anti-PVRIG antibody and/or antigen binding portion composition not containing the stabilizing agent or containing a lower amount of the stabilizing agent. In other embodiments, an anti-PVRIG antibody and/or antigen binding portion composition is considered to have been stabilized by the addition of a stabilizing agent (e.g., at least one amino acid, salt, and/or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage more anti-PVRIG antibody and/or antigen binding portion activity after storage for a period of time, as compared to an anti-PVRIG antibody and/or antigen binding portion composition not containing the stabilizing agent or containing a lower amount of the stabilizing agent.

In one embodiment, a stored anti-PVRIG antibody and/or antigen binding portion composition is considered stable as long as the percentage of anti-PVRIG antibody and/or antigen binding portion present in an aggregated state remains no more than 50%. In some embodiments, a stored anti-PVRIG antibody and/or antigen binding portion thereof composition is considered stable as long as the percentage of the anti-PVRIG antibody and/or antigen binding portion thereof present in an aggregated state remains no more than 45%, 40%, 35%, 30%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less.

In some embodiments, an anti-PVRIG antibody and/or antigen binding portion composition is considered to have been stabilized by the addition of a stabilizing agent (anti-PVRIG antibody and/or antigen binding portion composition, at least one amino acid, salt, and/or non-ionic surfactant) when the composition contains at least 10% less anti-PVRIG antibody and/or antigen binding portion present in an aggregated state after storage for a period of time, as compared to an anti-PVRIG antibody and/or antigen binding portion composition not containing the stabilizing agent or containing a lower amount of the stabilizing agent. In other embodiments, an anti-PVRIG antibody and/or antigen binding portion composition is considered to have been stabilized by the addition of a stabilizing agent (e.g., at least one amino acid, salt, and/or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage less anti-PVRIG antibody and/or antigen binding portion present in an aggregated state after storage for a period of time, as compared to an anti-PVRIG antibody and/or antigen binding portion composition not containing the stabilizing agent or containing a lower amount of the stabilizing agent

In some embodiments, a stored anti-PVRIG antibody and/or antigen binding portion composition composition is considered stable as long as the composition maintains at least 40% of the starting binding activity (e.g., at time=0) after being subjected to mechanical stress. In another embodiment, a stored composition is considered stable as long as the composition maintains 45%, 50%, 55%, 60%, 65%, 70%, 75%, 80%, 85%, 90%, 95% or more of the starting binding activity (e.g., at time=0) after being subjected to mechanical stress. In some embodiments, the mechanical stress is agitation (e.g., shaking).

In some embodiments, an anti-PVRIG antibody and/or antigen binding portion composition is considered to have been stabilized by the addition of a stabilizing agent (e.g., at least one amino acid, salt, or non-ionic surfactant) when the anti-PVRIG antibody and/or antigen binding portion composition contains at least 10% more binding activity after being subjected to mechanical stress, as compared to an anti-PVRIG antibody and/or antigen binding portion composition not containing the stabilizing agent or containing a lower amount of the stabilizing agent. In other embodiments, an anti-PVRIG antibody and/or antigen binding portion composition is considered to have been stabilized by the addition of a stabilizing agent (e.g., a sugar, sugar alcohol, or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage more furin activity after being subjected to mechanical stress, as compared to an anti-PVRIG antibody and/or antigen binding portion composition not containing the stabilizing agent or containing a lower amount of the stabilizing agent. In a specific embodiment, the mechanical stress is agitation (e.g., shaking).

In some embodiments, a stored anti-PVRIG antibody and/or antigen binding portion composition is considered stable as long as the percentage of anti-PVRIG antibody and/or antigen binding portion present in an aggregated state remains no more than 50% after being subjected to mechanical stress. In other embodiments, a stored anti-PVRIG antibody and/or antigen binding portion composition is considered stable as long as the percentage of anti-PVRIG antibody and/or antigen binding portion present in an aggregated state remains no more than 45%, 40%, 35%, 30%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less after being subjected to mechanical stress. In some embodiments, the mechanical stress is agitation (e.g., shaking).

In some embodiments, an anti-PVRIG antibody and/or antigen binding portion composition is considered to have been stabilized by the addition of a stabilizing agent (e.g., at least one amino acid, salt, or non-ionic surfactant) when the composition contains at least 10% less anti-PVRIG antibody and/or antigen binding portion present in an aggregated state after being subjected to mechanical stress, as compared to an anti-PVRIG antibody and/or antigen binding portion composition not containing the stabilizing agent or containing a lower amount of the stabilizing agent. In some embodiments, an anti-PVRIG antibody and/or antigen binding portion composition is considered to have been stabilized by the addition of a stabilizing agent (e.g., at least one amino acid, salt, or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage less anti-PVRIG antibody and/or antigen binding portion present in an aggregated state after being subjected to mechanical stress, as compared to an anti-PVRIG antibody and/or antigen binding portion composition not containing the stabilizing agent or containing a lower amount of the stabilizing agent. In a specific embodiment, the mechanical stress is agitation (e.g., shaking).

In some embodiments, the highly stabilized formulations of the invention have a shelf life of at least 6 months. As will be appreciated, this shelf life may be at frozen temperatures (i.e., −80° C., −25° C., 0° C.), refrigerated (0° C. to 10° C.), or room temperature (20° C. to 32° C.) in liquid or lyophilized form. In further aspects, the highly stabilized formulations of the invention have a shelf life of at least 12, 18, 24, 30, 36, 42, 48, 54, or 60 months.

In some embodiments, shelf life is determined by a percent activity remaining after storage at any of the above temperatures for any of the above periods of time. In some embodiments, shelf life means that the formulation retains at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100% of furin activity as measured by any of the assays described herein or known in the art as compared to activity prior to storage for any of the above amounts of time at any of the above temperatures.

In some embodiments, the present invention provides a stable liquid pharmaceutical formulation of an anti-PVRIG antibody comprising:

(a) an anti-PVRIG antibody, wherein the anti-PVRIG antibody comprises an antibody with CDRs identical to those shown in FIG. 3;

(b) 25 mM histidine;

(c) 60 mM NaCl;

(d) 100 mM L-Arginine, and

(e) 0.01% w/v polysorbate 80,

wherein the composition has a pH from 5.5 to 7.0.

In some embodiments, the anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL. In some embodiments, the anti-PVRIG antibody is at a concentration of from 10 mg/mL to 40 mg/mL. In some embodiments, the anti-PVRIG antibody is at a concentration of from 15 mg/mL to 40 mg/mL. In some embodiments, the anti-PVRIG antibody is at a concentration of from 15 mg/mL to 30 mg/mL. In some embodiments, the anti-PVRIG antibody is at a concentration of from 10 mg/mL to 25 mg/mL. In some embodiments, the anti-PVRIG antibody is at a concentration of from 15 mg/mL to 25 mg/mL. In some embodiments, the anti-PVRIG antibody is at a concentration of from 10 mg/mL to 25 mg/mL. In some embodiments, the anti-PVRIG antibody is at a concentration of from 15 mg/mL to 25 mg/mL. In some embodiments, the anti-PVRIG antibody is at a concentration of from 20 mg/mL to 25 mg/mL. In some embodiments, the anti-PVRIG antibody is at a concentration of about 20 mg/mL.

In some embodiments, the present invention provides a stable liquid pharmaceutical formulation of an anti-PVRIG antibody comprising:

(a) an anti-PVRIG antibody, wherein the anti-PVRIG antibody comprises an antibody with CDRs identical to those shown in FIG. 3;

(b) from 10 mM to 100 mM histidine;

(c) from 30 mM to 100 mM NaCl;

(d) from 20 mM to 150 mM L-Arginine, and

(e) from 0.005% to 0.1% w/v polysorbate 80 wherein the composition has a pH from 5.5 to 7.0.

B. Amino Acids

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising at least one amino acid. In some embodiments, the at least one amino acid is histidine. In some embodiments, the at least one amino acid is arginine. In some embodiments, the present invention provides a stable liquid pharmaceutical formulation of an anti-PVRIG antibody or antigen binding fragment thereof comprising at least two amino acids. In some embodiments, the at least two amino acids are histidine and arginine.

In some embodiments, the pharmaceutical formulation comprises from 10 mM to 80 mM histidine, from 15 mM to 70 mM histidine, from 20 mM to 60 mM histidine, from 20 mM to 50 mM histidine, or from 20 mM to 30 mM histidine. In some embodiments, the pharmaceutical formulation comprises from 10 mM to 80 mM histidine. In some embodiments, the pharmaceutical formulation comprises from 15 mM to 70 mM histidine. In some embodiments, the pharmaceutical formulation comprises from 20 mM to 60 mM histidine. In some embodiments, the pharmaceutical formulation comprises from from 20 mM to 50 mM histidine. In some embodiments, the pharmaceutical formulation comprises from 20 mM to 30 mM histidine. In some embodiments, the pharmaceutical formulation comprises about 25 mM histidine.

In some embodiments, the pharmaceutical formulation comprises from 10 mM to 80 mM histidine. In some embodiments, the pharmaceutical formulation comprises from 15 mM to 70 mM histidine. In some embodiments, the pharmaceutical formulation comprises from 20 mM to 60 mM histidine. In some embodiments, the pharmaceutical formulation comprises from 20 mM to 50 mM histidine. In some embodiments, the pharmaceutical formulation comprises from 20 mM to 30 mM histidine. In some embodiments, the pharmaceutical formulation comprises about 25 mM histidine.

In some embodiments, the pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine. In some embodiments, the pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine.

In some embodiments, the pharmaceutical formulation comprises from 20 mM to 140 mM L-arginine. In some embodiments, the pharmaceutical formulation comprises from 30 mM to 140 mM L-arginine. In some embodiments, the pharmaceutical formulation comprises from 40 mM to 130 mM L-arginine. In some embodiments, the pharmaceutical formulation comprises from 50 mM to 120 mM L-arginine. In some embodiments, the pharmaceutical formulation comprises from 60 mM to 110 mM L-arginine. In some embodiments, the pharmaceutical formulation comprises from 70 mM to 110 mM L-arginine. In some embodiments, the pharmaceutical formulation comprises from 80 mM to 110 mM L-arginine.

In some embodiments, the pharmaceutical formulation comprises from 90 mM to 110 mM L-arginine. In some embodiments, the pharmaceutical formulation comprises about 100 mM L-arginine.

C. Sugar and/or Sugar Alcohol

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising no sugar and/or sugar alcohol. In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising no sugar. In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising no sugar alcohol.

In some embodiments, the present invention provides a stable liquid pharmaceutical formulation of an anti-PVRIG antibody or antigen binding fragment thereof comprising a sugar and/or sugar alcohol. In some embodiments, the sugar is trehalose or sucrose. In some embodiments, the sugar is trehalose. In some embodiments, the sugar is sucrose. In some embodiments, the sugar is only one of trehalose or sucrose but not both.

In some embodiments, the sugar is in an amount of from about 0.5% to 10%, 1% to 9.5%, 1.5% to 9%, 2.0% to 8.5%, 2.5% to 8%, 3.0% to 7.5%, 3.5% to 7%, 4.0% to 6.5%, 4.5% to 6%, and/or 4.5% to 5.5%. In some embodiments, the sugar is in an amount of from about 0.5% to 10%. In some embodiments, the sugar is in an amount of from about 1% to 9.5%. In some embodiments, the sugar is in an amount of from about 1.5% to 9%. In some embodiments, the sugar is in an amount of from about 2.0% to 8.5%. In some embodiments, the sugar is in an amount of from about 2.5% to 8%. In some embodiments, the sugar is in an amount of from about 3.0% to 7.5%. In some embodiments, the sugar is in an amount of from about 3.5% to 7%. In some embodiments, the sugar is in an amount of from about 4.0% to 6.5%. In some embodiments, the sugar is in an amount of from about 4.5% to 6%. In some embodiments, the sugar is in an amount of from about 4.5% to 5.5%. In some embodiments, the sugar is in an amount of about 5%

D. Non-Ionic Surfactants

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising a non-ionic surfactant. In some embodiments, the storage stable compositions of an anti-PVRIG antibody or antigen binding fragment comprise a non-ionic surfactant selected from a non-ionic water soluble monoglyceride, a non-ionic water soluble diglyceride, a non-ionic water soluble triglyceride, a non-ionic water soluble monofatty acid esters of polyethyelene glycol, a non-ionic water soluble difatty acid esters of polyethyelene glycol, a non-ionic water soluble sorbitan fatty acid ester, a non-ionic polyglycolyzed glyceride, a non-ionic water soluble triblock copolymer, and a combination thereof. In some embodiments, the non-ionic surfactant is polysorbate 80 (polyoxyethylene (20) sorbitan monooleate).

In some embodiments, the stable liquid pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, or from 0.009% to 0.05% w/v polysorbate 80. In some embodiments, the stable liquid pharmaceutical formulation comprises from 0.006% to 0.1% w/v polysorbate 80. In some embodiments, the stable liquid pharmaceutical formulation comprises from 0.007% to 0.09% w/v polysorbate 80. In some embodiments, the stable liquid pharmaceutical formulation comprises from 0.008% to 0.08% w/v polysorbate 80. In some embodiments, the stable liquid pharmaceutical formulation comprises from 0.009% to 0.09% w/v polysorbate 80. In some embodiments, the stable liquid pharmaceutical formulation comprises from 0.01% to 0.08% w/v polysorbate 80. In some embodiments, the stable liquid pharmaceutical formulation comprises from 0.01% to 0.07% w/v polysorbate 80. In some embodiments, the stable liquid pharmaceutical formulation comprises from 0.01% to 0.07% w/v polysorbate 80. In some embodiments, the stable liquid pharmaceutical formulation comprises from 0.01% to 0.06% w/v polysorbate 80. In some embodiments, the stable liquid pharmaceutical formulation comprises from 0.009% to 0.05% w/v polysorbate 80. In some embodiments, the stable liquid pharmaceutical formulation comprises about 0.01% polysorbate 80.

E. Pharmaceutically Acceptable Salts

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising a salt, for example, a pharmaceutically acceptable salt.

In some embodiments, the stable liquid pharmaceutical formulation comprising an anti-PVRIG antibody or antigen binding fragment thereof provided herein include a pharmaceutically acceptable salt at a concentration tolerated by the an anti-PVRIG antibody or antigen binding fragment thereof during storage. In some embodiments, the pharmaceutically acceptable salt is a chloride salt. In some embodiments, the pharmaceutically acceptable salt is a monovalent chloride salt. In a more specific embodiment, the pharmaceutically acceptable salt is sodium chloride, potassium chloride, or a combination thereof.

In some embodiments, the stable liquid pharmaceutical formulation comprises from 30 mM to 100 mM NaCl, from 30 mM to 90 mM NaCl, from 40 mM to 80 mM NaCl, from 30 mM to 70 mM histidine, or from 45 mM to 70 mM NaCl.

In some embodiments, the stable liquid pharmaceutical formulation comprises from 30 mM to 100 mM NaCl. In some embodiments, the stable liquid pharmaceutical formulation comprises from 30 mM to 90 mM NaCl. In some embodiments, the stable liquid pharmaceutical formulation comprises from 40 mM to 80 mM NaCl. In some embodiments, the stable liquid pharmaceutical formulation comprises from 30 mM to 70 mM histidine. In some embodiments, the stable liquid pharmaceutical formulation comprises or from 45 mM to 70 mM NaCl. In some embodiments, pharmaceutical formulation comprises about 60 mM NaCl.

F. Buffering Agents

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) that is buffered at a physiologically acceptable pH. In some embodiments, the physiologically acceptable pH is from about 6.0 to about 7.0.

In some embodiments, stable liquid pharmaceutical formulation of an anti-PVRIG antibody or antigen binding fragment thereof has a pH of from 6 to 7.0. In some embodiments, stable liquid pharmaceutical formulation of an anti-PVRIG antibody or antigen binding fragment thereof has a pH of 6, 6.1, 6.2, 6.3, 6.4, 6.5, 6.6, 6.7, 6.8, 6.9, or 7.0. In some embodiments, the pH is from 6.1 to 6.9. In some embodiments, the pH is from 6.2 to 6.9. In some embodiments, the pH is from 6.3 to 6.8. In some embodiments, the pH is from 6.3 to 6.7. In some embodiments, the pH is from 6.4 to 6.8. In some embodiments, the pH is from 6.5 to 6.8. In some embodiments, the pH is from 6.6 to 6.8. In some embodiments, the pH is 6.3, 6.4, 6.5, 6.6, or 6.7. In some embodiments, the pH is 6.5+/−0.2.

G. Methods for Diluting Aqueous Compositions

[In some embodiments, the method includes adding a dilution buffer, to form a diluted stable liquid pharmaceutical formulation comprising an anti-PVRIG antibody or antigen binding fragment thereof (e.g., anti-PVRIG antibodies including those with CDRs identical to those shown in FIG. 3). In some embodiments, the dilution buffer is added at a ratio of from 1:1 (dilution buffer:formulation) to 1000:1 (dilution buffer:formulation). In another embodiment, the dilution buffer is added at a ratio of from 1:1 dilution buffer:formulation) to 500:1 (dilution buffer:formulation). In another embodiment, the dilution buffer is added at a ratio of from 1:1 (dilution buffer:formulation) to 250:1 (dilution buffer:formulation). In another embodiment, the dilution buffer is added at a ratio of from 1:1 (dilution buffer:formulation) to 200:1 (dilution buffer:formulation). In another embodiment, the dilution buffer is added at a ratio of from 1:1 (dilution buffer:formulation) to 100:1 (dilution buffer:formulation). In another embodiment, the dilution buffer is added at a ratio of from 1:1 (dilution buffer:formulation) to 50:1 (dilution buffer:formulation).

In some embodiments, the stable liquid pharmaceutical formulation comprising an anti-PVRIG antibody or antigen binding fragment thereof is diluted from 1-fold to 1000-fold, from 1-fold to 500-fold, from 1-fold to 250-fold, from 1-fold to 200-fold, from 1-fold to 100-fold, from 1-fold to 50-fold, from 1-fold to 10-fold, from 10-fold to 1000-fold, from 10-fold to 500-fold, from 10-fold to 250-fold, from 10-fold to 200-fold, from 10-fold to 100-fold, from 10-fold to 50-fold, from 50-fold to 1000-fold, from 50-fold to 500-fold, from 50-fold to 250-fold, from 50-fold to 200-fold, from 50-fold to 100-fold, from 100-fold to 1000-fold, from 100-fold to 500-fold, from 100-fold to 250-fold, from 100-fold to 200-fold, from 200-fold to 1,000-fold, from 200-fold to 500-fold, or from 200-fold to 250-fold.

H. Stability Assays

As discussed herein, the stable liquid pharmaceutical formulations comprising an anti-PVRIG antibody or antigen binding fragment thereof (e.g., anti-PVRIG antibodies including those with CDRs identical to those shown in FIG. 3) show improved stability as compared to control formulations. In one embodiment, improved stability includes retention of a higher percentage of binding activity and/or no reduction in binding activity as compared to control formulations in various stability assays. Such assays can be used to determine if a formulation is a highly stabilized formulation. In some embodiments, the highly stabilized formulation has at least 5%, 10%, 20%, 30%, 40%, 50%, 55%, 60%, 65%, 70%, 75%, 80%, 85%, 90%, 95%, 96%, 97%, 98%, 99% or greater activity than a control formulation when assessed by any of the stability assays discussed herein or known in the art.

In some embodiments, the liquid pharmaceutical formulations comprising an anti-PVRIG antibody or antigen binding fragment thereof are tested under stressor conditions, such as storage at high temperature, agitation, freeze/thaw cycles, or some combination thereof. After such stressors, the formulations are assayed using any of the methods described herein or known in the art to determine the stability under these conditions.

### A280 and Appearance Analysis

In some embodiments, an A280 by SoloVPE assay is used to examine the appearance of the stable liquid pharmaceutical formulations comprising an anti-PVRIG antibody or antigen binding fragment thereof.

In some embodiments, the SoloVPE assay can be employed to examine concentrations for the stable liquid pharmaceutical formulations comprising an anti-PVRIG antibody or antigen binding fragment thereof.

A280: Amino acids containing aromatic side chains exhibit strong UV-light absorption at the wavelength of 280 nm. Once an absorptivity coefficient has been established for a given protein, the protein's concentration in solution can be calculated from its absorbance. The method is designed to determine the protein concentration by measuring its absorbance at 280 nm using the SoloVPE instrument without dilution (https://www.ctechnologiesinc.com/products/solovpe)

Appearance: Sample appearance determination is assessed by holding the sample within a controlled light source and observe the appearance of the material. Gently agitate the solution and determine if the appearance changes when viewed against a black and white background. Use adjectives such as “clear”, “turbid”, or “slightly turbid” to assess clarity. Be specific with regards to the color of the material. If the material is colorless then state that as a result (i.e. clear, colorless solution). specify the physical state of the sample (i.e. liquid or frozen liquid)

### Binding Assay Analysis

In some embodiments, a binding assay can be performed to examine the activity of the the stable liquid pharmaceutical formulations comprising an anti-PVRIG antibody or antigen binding fragment thereof.

### LabChip Analysis

In some embodiments, a LabChip analysis is employed to examine purity, including for example, IgG purity as well as HC+LC percentages for the stable liquid pharmaceutical formulations comprising an anti-PVRIG antibody or antigen binding fragment thereof. In some embodiments, the stable liquid pharmaceutical formulations comprising an anti-PVRIG antibody or antigen binding fragment thereof exhibit IgG purity percentages greater than 94%, greater than 95%, greater than 96%, greater than 97%, or greater than 98%. In some embodiments, the stable liquid pharmaceutical formulations comprising an anti-PVRIG antibody or antigen binding fragment thereof exhibit IgG purity percentages were from about 95% to 98%. In some embodiments, the stable liquid pharmaceutical formulations comprising an anti-PVRIG antibody or antigen binding fragment thereof exhibit IgG purity percentages from about 96% to 97%. In some embodiments, the stable liquid pharmaceutical formulations comprising an anti-PVRIG antibody or antigen binding fragment thereof exhibit HC+LC percentages from about 96% to 100%. In some embodiments, the stable liquid pharmaceutical formulations comprising an anti-PVRIG antibody or antigen binding fragment thereof exhibit HC+LC percentages from about 97% to 100%. In some embodiments, the stable liquid pharmaceutical formulations comprising an anti-PVRIG antibody or antigen binding fragment thereof exhibit HC+LC percentages from about 98% to 100%.

cIEF Analysis

In some embodiments, a capillary isoelectric focusing (cIEF) can be employed to analyze the stable liquid pharmaceutical formulations comprising an anti-PVRIG antibody or antigen binding fragment thereof for the presence of additional species, including for example, minor acidic species.

### MFI Analysis

Antibodies can form sub-visible particles in response to stressed conditions, such as heat, freeze/thaw cycles, and agitation. In some embodiments, a microflow imaging (MFI) analysis can be employed to analyze the stable liquid pharmaceutical formulations comprising an anti-PVRIG antibody or antigen binding fragment thereof for the formation of particles in response to stressed conditions. In some embodiments, the stable liquid pharmaceutical formulations of the anti-PVRIG antibody or antigen binding fragment thereof provide for a formulation capable of stabilizing the anti-PVRIG antibody or antigen binding fragment thereof against these stressed conditions and protecting against the formation of particles. MFI can be used to evaluate particle counts at different size ranges (<2 μm, <5 μm, <10 μm, and <25 μm) in different formulations under stressed conditions. Typically, MFI data can be evaluated to choose an appropriate formulation based on generation of the lowest amount of particles/mL for all sizes of particles across all time points, conditions, and formulations.

### SEC Analysis

In some embodiments, size exclusion chromatography (SEC) can be employed to analyze the stable liquid pharmaceutical formulations comprising an anti-PVRIG antibody or antigen binding fragment thereof. The SEC data showed HMW throughout all time points and conditions; however, it remained stable at about 1%. LMW was present in accelerated conditions and 2-8° C. 8 week time point. Within the 40° C. condition, the LMW did increase from about 1% to 3% from Week 1 to Week 2.

### I. Selected Formulation Embodiments

In some embodiments, the present invention provides a stable liquid pharmaceutical formulation of an anti-PVRIG antibody comprising:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides a stable liquid pharmaceutical formulation of an anti-PVRIG antibody comprising:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9).

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides a stable liquid pharmaceutical formulation comprising:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides a stable liquid pharmaceutical formulation comprising:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides a stable liquid pharmaceutical formulation comprising:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides a stable liquid pharmaceutical formulation comprising:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides a stable liquid pharmaceutical formulation comprising:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides a stable liquid pharmaceutical formulation comprising:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

### VI. Administration of Formulations of Anti-PVRIG Antibodies

Administration of the pharmaceutical composition comprising anti-PVRIG antibodies of the present invention (e.g., anti-PVRIG antibodies including those with CDRs identical to those shown in FIG. 3), preferably in the form of a sterile aqueous solution, may be done in a variety of ways, As is known in the art, protein therapeutics are often delivered by IV infusion. The antibodies of the present invention may also be delivered using such methods. For example, administration may venious be by intravenous infusion with 0.9% sodium chloride as an infusion vehicle. Such techniques are disclosed in Remington's Pharmaceutical Sciences 16th edition, Osol, A. Ed., 1980.

The dosing amounts and frequencies of administration are, in some embodiments, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. In order to treat a patient, a therapeutically effective dose of the Fc variant of the present invention may be administered. By “therapeutically effective dose” herein is meant a dose that produces the effects for which it is administered.

### VII. Dosing

In some embodiments, the anti-PVRIG antibody and/or antigen binding portion thereof formulations of the present invention can be formulated for administration, including as a unit dosage formulation. In some embodiments, the anti-PVRIG antibody and/or antigen binding portion thereof formulations are administered at a dosage of 0.01 mg/kg of the anti-PVRIG antibody and/or antigen binding portion thereof. In some embodiments, the anti-PVRIG antibody and/or antigen binding portion thereof formulations are administered at a dosage of 0.02 mg/kg of the anti-PVRIG antibody and/or antigen binding portion thereof. In some embodiments, the anti-PVRIG antibody and/or antigen binding portion thereof formulations are administered at a dosage of 0.03 mg/kg of the anti-PVRIG antibody and/or antigen binding portion thereof. In some embodiments, the anti-PVRIG antibody and/or antigen binding portion thereof formulations are administered at a dosage of 0.04 mg/kg of the anti-PVRIG antibody and/or antigen binding portion thereof. In some embodiments, the anti-PVRIG antibody and/or antigen binding portion thereof formulations are administered at a dosage of 0.05 mg/kg of the anti-PVRIG antibody and/or antigen binding portion thereof. In some embodiments, the anti-PVRIG antibody and/or antigen binding portion thereof formulations are administered at a dosage of 0.06 mg/kg of the anti-PVRIG antibody and/or antigen binding portion thereof. In some embodiments, the anti-PVRIG antibody and/or antigen binding portion thereof formulations are administered at a dosage of 0.07 mg/kg of the anti-PVRIG antibody and/or antigen binding portion thereof. In some embodiments, the anti-PVRIG antibody and/or antigen binding portion thereof formulations are administered at a dosage of 0.08 mg/kg of the anti-PVRIG antibody and/or antigen binding portion thereof. In some embodiments, the anti-PVRIG antibody and/or antigen binding portion thereof formulations are administered at a dosage of 0.09 mg/kg of the anti-PVRIG antibody and/or antigen binding portion thereof. In some embodiments, the anti-PVRIG antibody and/or antigen binding portion thereof formulations are administered at a dosage of 0.1 mg/kg of the anti-PVRIG antibody and/or antigen binding portion thereof. In some embodiments, the anti-PVRIG antibody and/or antigen binding portion thereof formulations are administered at a dosage of 0.2 mg/kg of the anti-PVRIG antibody and/or antigen binding portion thereof. In some embodiments, the anti-PVRIG antibody and/or antigen binding portion thereof formulations are administered at a dosage of 0.3 mg/kg of the anti-PVRIG antibody and/or antigen binding portion thereof. In some embodiments, the anti-PVRIG antibody and/or antigen binding portion thereof formulations are administered at a dosage of 0.5 mg/kg of the anti-PVRIG antibody and/or antigen binding portion thereof. In some embodiments, the anti-PVRIG antibody and/or antigen binding portion thereof formulations are administered at a dosage of 0.8 mg/kg of the anti-PVRIG antibody and/or antigen binding portion thereof. In some embodiments, the anti-PVRIG antibody and/or antigen binding portion thereof formulations are administered at a dosage of 1 mg/kg of the anti-PVRIG antibody and/or antigen binding portion thereof. In some embodiments, the anti-PVRIG antibody and/or antigen binding portion thereof formulations are administered at a dosage of 2 mg/kg of the anti-PVRIG antibody and/or antigen binding portion thereof. In some embodiments, the anti-PVRIG antibody and/or antigen binding portion thereof formulations are administered at a dosage of 3 mg/kg of the anti-PVRIG antibody and/or antigen binding portion thereof. In some embodiments, the anti-PVRIG antibody and/or antigen binding portion thereof formulations are administered at a dosage of 4 mg/kg of the anti-PVRIG antibody and/or antigen binding portion thereof. In some embodiments, the anti-PVRIG antibody and/or antigen binding portion thereof formulations are administered at a dosage of 5 mg/kg of the anti-PVRIG antibody and/or antigen binding portion thereof. In some embodiments, the anti-PVRIG antibody and/or antigen binding portion thereof formulations are administered at a dosage of 6 mg/kg of the anti-PVRIG antibody and/or antigen binding portion thereof. In some embodiments, the anti-PVRIG antibody and/or antigen binding portion thereof formulations are administered at a dosage of 7 mg/kg of the anti-PVRIG antibody and/or antigen binding portion thereof. In some embodiments, the anti-PVRIG antibody and/or antigen binding portion thereof formulations are administered at a dosage of 8 mg/kg of the anti-PVRIG antibody and/or antigen binding portion thereof. In some embodiments, the anti-PVRIG antibody and/or antigen binding portion thereof formulations are administered at a dosage of 9 mg/kg of the anti-PVRIG antibody and/or antigen binding portion thereof. In some embodiments, the anti-PVRIG antibody and/or antigen binding portion thereof formulations are administered at a dosage of 10 mg/kg of the anti-PVRIG antibody and/or antigen binding portion thereof. In some embodiments, the anti-PVRIG antibody and/or antigen binding portion thereof formulations are administered at a dosage of 20 mg/kg of the anti-PVRIG antibody and/or antigen binding portion thereof.

In some embodiments, the anti-PVRIG antibody and/or antigen binding portion thereof formulations is administered at a dosage of about 0.01 mg/kg to about 20 mg/kg of the anti-PVRIG antibody. In some embodiments, the anti-PVRIG antibody and/or antigen binding portion thereof formulations is administered at a dosage of about 0.01 mg/kg to about 10 mg/kg of the anti-PVRIG antibody. In some embodiments, the anti-PVRIG antibody and/or antigen binding portion thereof formulations is administered at a dosage of about 20 mg/kg. In some embodiments, the anti-PVRIG antibody and/or antigen binding portion thereof formulations is administered at a dosage of about 20 mg/kg each 4 weeks. In some embodiments, the anti-PVRIG antibody and/or antigen binding portion thereof formulations is administered at a dosage of about 20 mg/kg IV each 4 weeks. In some embodiments, formulation is administered at a dosage of about 0.1 mg/kg to about 10 mg/kg of the anti-PVRIG antibody. In some embodiments, formulation is administered at a dosage of about 1 mg/kg to about 10 mg/kg of the anti-PVRIG antibody. In some embodiments, formulation is administered at a dosage of about 2 mg/kg to about 10 mg/kg of the anti-PVRIG antibody. In some embodiments, formulation is administered at a dosage of about 3 mg/kg to about 10 mg/kg of the anti-PVRIG antibody. In some embodiments, formulation is administered at a dosage of about 4 mg/kg to about 10 mg/kg of the anti-PVRIG antibody. In some embodiments, formulation is administered at a dosage of about 5 mg/kg to about 10 mg/kg of the anti-PVRIG antibody. In some embodiments, formulation is administered at a dosage of about 5 mg/kg to about 10 mg/kg of the anti-PVRIG antibody. In some embodiments, formulation is administered at a dosage of about 7 mg/kg to about 10 mg/kg of the anti-PVRIG antibody. In some embodiments, formulation is administered at a dosage of about 8 mg/kg to about 10 mg/kg of the anti-PVRIG antibody. In some embodiments, formulation is administered at a dosage of about 9 mg/kg to about 10 mg/kg of the anti-PVRIG antibody. In some embodiments, formulation is administered at a dosage of about 0.01 mg/kg, 0.03 mg/kg, 0.1 mg/kg, 0.3 mg/kg, 1 mg/kg, 3 mg/kg, 10 mg/kg or 20 mg/kg of the anti-PVRIG antibody.

A. Selected Dosing with Formulation Embodiments

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg or 20 mg/kg, and wherein the stable liquid formulation of the anti-PVRIG antibody comprises:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9).

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments of the stable liquid pharmaceutical formulation, the formulation is administered with an anti-PD-1 antibody.

In some embodiments of the stable liquid pharmaceutical formulation, the anti-PD-1 antibody is an antibody selected from the group consisting of pembrolizumab and nivolumab.

In some embodiments of the stable liquid pharmaceutical formulation, the anti-PD-1 antibody is nivolumab. In some embodiments of the stable liquid pharmaceutical formulation, the anti-PD-1 antibody is nivolumab is administered at a dosage of about 360 mg or 480 mg. In some embodiments of the stable liquid pharmaceutical formulation, the anti-PD-1 antibody is nivolumab is administered at a dosage of about 360 mg. In some embodiments of the stable liquid pharmaceutical formulation, the anti-PD-1 antibody is nivolumab is administered at a dosage of about 480 mg.

In some embodiments of the stable liquid pharmaceutical formulation, the anti-PD-1 antibody is pembrolizumab.

### VIII. Methods of Using the Anti-PVRIG Antibody Formulations

A. Therapeutic Uses

The anti-PVRIG antibodies (e.g., anti-PVRIG antibodies including those with CDRs identical to those shown in FIG. 3) find use in treating patients, such as human subjects, generally with a condition associated with PVRIG. The term “treatment” as used herein, refers to both therapeutic treatment and prophylactic or preventative measures, which in this example relates to treatment of cancer; however, also as described below, uses of antibodies and pharmaceutical compositions are also provided for treatment of infectious disease, sepsis, and/or autoimmune conditions, and/or for inhibiting an undesirable immune activation that follows gene therapy. Those in need of treatment include those already with cancer as well as those in which the cancer is to be prevented. Hence, the mammal to be treated herein may have been diagnosed as having the cancer or may be predisposed or susceptible to the cancer. As used herein the term “treating” refers to preventing, delaying the onset of, curing, reversing, attenuating, alleviating, minimizing, suppressing, halting the deleterious effects or stabilizing of discernible symptoms of the above-described cancerous diseases, disorders or conditions. It also includes managing the cancer as described above. By “manage” it is meant reducing the severity of the disease, reducing the frequency of episodes of the disease, reducing the duration of such episodes, reducing the severity of such episodes, slowing/reducing cancer cell growth or proliferation, slowing progression of at least one symptom, amelioration of at least one measurable physical parameter and the like. For example, immunostimulatory anti-PVRIG immune molecules should promote T cell or NK or cytokine immunity against target cells, e.g., cancer, infected or pathogen cells and thereby treat cancer or infectious diseases by depleting the cells involved in the disease condition. Conversely, immunoinhibitory anti-PVRIG immune molecules should reduce T cell or NK activity and/or or the secretion of proinflammatory cytokines which are involved in the disease pathology of some immune disease such as autoimmune, inflammatory or allergic conditions and thereby treat or ameliorate the disease pathology and tissue destruction that may be associated with such conditions (e.g., joint destruction associated with rheumatoid arthritis conditions).

The PVRIG antibodies of the invention are provided in therapeutically effective dosages. A “therapeutically effective dosage” of an anti-PVRIG immune molecule according to at least some embodiments of the present invention preferably results in a decrease in severity of disease symptoms, an increase in frequency and duration of disease symptom-free periods, an increase in lifespan, disease remission, or a prevention or reduction of impairment or disability due to the disease affliction. For example, for the treatment of PVRIG positive tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject.

One of ordinary skill in the art would be able to determine a therapeutically effective amount based on such factors as the subject's size, the severity of the subject's symptoms, and the particular composition or route of administration selected.

1. Cancer Treatment

The PVRIG antibody formulations of the invention find particular use in the treatment of cancer. In general, the antibodies of the invention are immunomodulatory, in that rather than directly attack cancerous cells, the anti-PVRIG antibodies of the invention stimulate the immune system, generally by inhibiting the action of PVRIG. Thus, unlike tumor-targeted therapies, which are aimed at inhibiting molecular pathways that are crucial for tumor growth and development, and/or depleting tumor cells, cancer immunotherapy is aimed to stimulate the patient's own immune system to eliminate cancer cells, providing long-lived tumor destruction. Various approaches can be used in cancer immunotherapy, among them are therapeutic cancer vaccines to induce tumor-specific T cell responses, and immunostimulatory antibodies (i.e. antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients respond (as opposed to conventional or targeted therapy, where a large number of patients respond, but responses are transient).

By the time a tumor is detected clinically, it has already evaded the immune-defense system by acquiring immunoresistant and immunosuppressive properties and creating an immunosuppressive tumor microenvironment through various mechanisms and a variety of immune cells.

Accordingly, the anti-PVRIG antibodies of the invention are useful in treating cancer. Due to the nature of an immuno-oncology mechanism of action, PVRIG does not necessarily need to be overexpressed on or correlated with a particular cancer type; that is, the goal is to have the anti-PVRIG antibodies de-suppress T cell and NK cell activation, such that the immune system will go after the cancers.

“Cancer,” as used herein, refers broadly to any neoplastic disease (whether invasive or metastatic) characterized by abnormal and uncontrolled cell division causing malignant growth or tumor (e.g., unregulated cell growth). The term “cancer” or “cancerous” as used herein should be understood to encompass any neoplastic disease (whether invasive, non-invasive or metastatic) which is characterized by abnormal and uncontrolled cell division causing malignant growth or tumor, non-limiting examples of which are described herein. This includes any physiological condition in mammals that is typically characterized by unregulated cell growth.

In some embodiments, the anti-PVRIG formulations of the present invention can be used in the treatment of solid tumors (including, for example, cancers of the lung, liver, breast, brain, GI tract) and blood cancers (including for example, leukemia and preleukemic disorders, lymphoma, plasma cell disorders) carcinoma, lymphoma, blastoma, sarcoma, and leukemia or lymphoid malignancies. In some embodiments, the cancer is early. In some embodiments, the cancer is advanced (including metastatic). In some embodiments, the cancers amenable for treatment of the invention include cancers that express or do not express PVRIG and further include non-metastatic or non-invasive, as well as invasive or metastatic cancers, including cancers where PVRIG expression by immune, stromal, or diseased cells suppresses antitumor responses and anti-invasive immune responses. In some embodiments, the anti-PVRIG formulations can be used for the treatment of vascularized tumors. In some embodiments, the cancer for treatment using the anti-PVRIG formulations of the present invention includes carcinoma, lymphoma, sarcoma, and/or leukemia. In some embodiments, the cancer for treatment using the anti-PVRIG formulations of the present invention includes melanoma, non-melanoma skin cancer (squamous and basal cell carcinoma), mesothelioma, squamous cell cancer, lung cancer (including small-cell lung cancer, non-small cell lung cancer, soft-tissue sarcoma, Kaposi's sarcoma, adenocarcinoma of the lung, squamous carcinoma of the lung, cancer of the peritoneum, esophageal cancer, hepatocellular cancer, liver cancer (including HCC), gastric cancer, stomach cancer (including gastrointestinal cancer), pancreatic cancer, glioblastoma, cervical cancer, ovarian cancer, urothelial cancer, bladder cancer, hepatoma, glioma, brain cancer (as well as edema, such as that associated with brain tumors), breast cancer (including, for example, triple negative breast cancer), testis cancer, testicular germ cell tumors, colon cancer, colorectal cancer (CRC), colorectal cancer MSS (MSS-CRC; including refractory MSS colorectal; MSS=microsatellite stable status), primary peritoneal cancer, microsatellite stable primary peritoneal cancer, platinum resistant microsatellite stable primary peritoneal cancer, CRC (MSS unknown), rectal cancer, endometrial cancer (including endometrial carcinoma), uterine carcinoma, salivary gland carcinoma, kidney cancer, renal cell cancer (RCC), renal cell carcinoma (RCC), prostate cancer, vulval cancer, thyroid cancer, hepatic carcinoma, carcinoid carcinoma, head and neck cancer, B-cell lymphoma (including non-Hodgkin's lymphoma, as well as low grade/follicular non-Hodgkin's lymphoma (NHL), small lymphocytic (SL) NHL, intermediate grade/follicular NHL, intermediate grade diffuse NHL, Diffuse Large B cell lymphoma, high grade immunoblastic NHL, high grade lymphoblastic NHL, high grade small non-cleaved cell NHL, bulky disease NHL, mantle cell lymphoma, AIDS-related lymphoma, and Waldenström's Macroglobulinemia), Hodgkin's lymphoma (HD), chronic lymphocytic leukemia (CLL), acute lymphoblastic leukemia (ALL), T cell Acute Lymphoblastic Leukemia (T-ALL), Acute myeloid leukemia (AML), Hairy cell leukemia, chronic myeloblastic leukemia, multiple myeloma, post-transplant lymphoproliferative disorder (PTLD), abnormal vascular proliferation associated with phakomatoses, Meigs' syndrome, Merkel Cells cancer, MSI-high cancer, KRAS mutant tumors, adult T-cell leukemia/lymphoma, adenoid cystic cancer (including adenoid cystic carcinoma), malignant melanoma, pancreatic cancer, pancreatic adenocarcinoma, ovarian cancer (including ovarian carcinoma), pleural mesothelioma, neuroendocrine lung cancer (including pleural mesothelioma, neuroendocrine lung carcinoma), NSCL (large cell), NSCLC large cell adenocarcinoma, non-small cell lung carcinoma (NSCLC), NSCLC squamous cell, cervical squamous cell carcinoma (cervical SCC), anal squamous cell carcinoma (anal SCC), neuroendocrine lung carcinoma, carcinoma of unknown primary, gallbladder cancer, malignant melanoma, pleural mesothelioma, and/or Myelodysplastic syndromes (MDS).

In some embodiments, the cancer for treatment using the anti-PVRIG formulations of the present invention includes a cancer selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma)breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, NSCLC, adenoid cystic cancer (including adenoid cystic carcinoma), primary peritoneal cancer, microsatellite stable primary peritoneal cancer, platinum resistant microsatellite stable primary peritoneal cancer, and/or Myelodysplastic syndromes (MDS).

“Cancer therapy” herein refers to any method that prevents or treats cancer or ameliorates one or more of the symptoms of cancer. Typically such therapies will comprises administration of immunostimulatory anti-PVRIG antibodies (including antigen-binding fragments) either alone or in combination with chemotherapy or radiotherapy or other biologics and for enhancing the activity thereof, i.e., in individuals wherein expression of PVRIG suppresses antitumor responses and the efficacy of chemotherapy or radiotherapy or biologic efficacy.

In some embodiments, anti-PVRIG antibodies are used in combination with antagonistic antibodies targeting PD-1 (e.g., anti-PD-1 antibodies), including for example but not limited to nivolumab and/or pembrolizumab. In some embodiments, the anti-PD-1 antibody is an antibody selected from the group consisting of nivolumab and pembrolizumab. In some embodiments, the anti-PD-1 antibody is nivolumab. In some embodiments, the anti-PD-1 antibody is pembrolizumab. In some embodiments, the anti-PD-1 antibody is nivolumab is administered at 360 mg. In some embodiments, the anti-PD-1 antibody is nivolumab is administered at 360 mg IV. In some embodiments, the anti-PD-1 antibody is nivolumab is administered at 360 mg IV 3 weeks (e.g., 360 mg IV Q-3 weeks). In some embodiments, the anti-PD-1 antibody is nivolumab is administered at 480 mg. In some embodiments, the anti-PD-1 antibody is nivolumab is administered at 480 mg IV. In some embodiments, the anti-PD-1 antibody is nivolumab is administered at 480 mg IV 3 weeks (e.g., 480 mg IV Q-3 weeks). In some embodiments, the anti-PD-1 antibody nivolumab is administered at 360 mg and the anti-PVRIG is administered at 20 mg/kg. In some embodiments, the anti-PD-1 antibody nivolumab is administered at 360 mg IV and the anti-PVRIG is administered at 20 mg/kg IV. In some embodiments, the anti-PD-1 antibody nivolumab is administered at 480 mg and the anti-PVRIG is administered at 20 mg/kg. In some embodiments, the anti-PD-1 antibody nivolumab is administered at 480 mg IV and the anti-PVRIG is administered at 20 mg/kg IV. In some embodiments, the anti-PD-1 antibody nivolumab is administered at 360 mg IV for 3 weeks (e.g., 360 mg IV Q-3 weeks) and the anti-PVRIG is administered at 20 mg/kg IV for 3 weeks. In some embodiments, the anti-PD-1 antibody nivolumab is administered at 480 mg IV for 3 weeks (e.g., 480 mg IV Q-3 weeks) and the anti-PVRIG is administered at 20 mg/kg for 3 weeks. In some embodiments, the anti-PD-1 antibody nivolumab is administered at 360 mg IV for 4 weeks (e.g., 360 mg IV Q-4 weeks) and the anti-PVRIG is administered at 20 mg/kg IV for 4 weeks. In some embodiments, the anti-PD-1 antibody nivolumab is administered at 480 mg IV for 4 weeks (e.g., 480 mg IV Q-4 weeks) and the anti-PVRIG is administered at 20 mg/kg for 4 weeks. In some embodiments the anti-PVRIG is CHA.7.518.1.H4(S241P). In some embodiments, the subject administered the anti-PVRIG antibody in combination with the anti-PD-1 antibody has exhausted all available standard therapy, including for example, but not limited to ECOG 0-1, prior anti-PD-1, prior anti-PD-L1, prior anti-CTLA-4, prior OX-40, and/or prior CD137 therapies.

2. Selected Monotherapy Treatment with Formulation Embodiments

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the v1CDR1, vlCDR2, and vlCDR3 from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9).

(a) an anti-PVRIG antibody;

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

3. Selected Combination Treatment with Formulation Embodiments

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9).

(a) an anti-PVRIG antibody;

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9).

(a) an anti-PVRIG antibody;

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9).

(a) an anti-PVRIG antibody;

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) heavy chain variable domain is from the heavy chain of CHA.7.518.1.H4(S241P) (SEQ ID NO:4), and
ii) a light chain variable domain is from the light chain of CHA.7.518.1.H4(S241P) (SEQ ID NO:9);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising:
   a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
ii) a light chain comprising:
   a) a VL-CL, wherein the VL from CHA.7.518.1.H4(S241P) (SEQ ID NO:9) and wherein the CL region is from human kappa 2 light chain;

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) from 10 mM to 100 mM histidine;

(c) from 30 mM to 100 mM NaCl;

(d) from 20 mM to 150 mM L-Arginine; and

(e) from 0.005% to 0.1% w/v polysorbate 80,

wherein the composition has a pH from 5.5 to 7.0.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:

(a) an anti-PVRIG antibody comprising:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);

(b) about 25 mM histidine;

(c) about 60 mM NaCl;

(d) about 100 mM L-Arginine; and

(e) about 0.01% % w/v polysorbate 80,

wherein the composition has a pH from 6.5+/−0.2.

### EXAMPLES

### Example 1: PVRIG Antibody Formulation Testing

The PVRIG antibody that was formulated in each buffer (A and B) was spiked with the corresponding excipients to create the 20 conditions listed in FIG. 6. Each formulation was referred to by the Formulation ID.

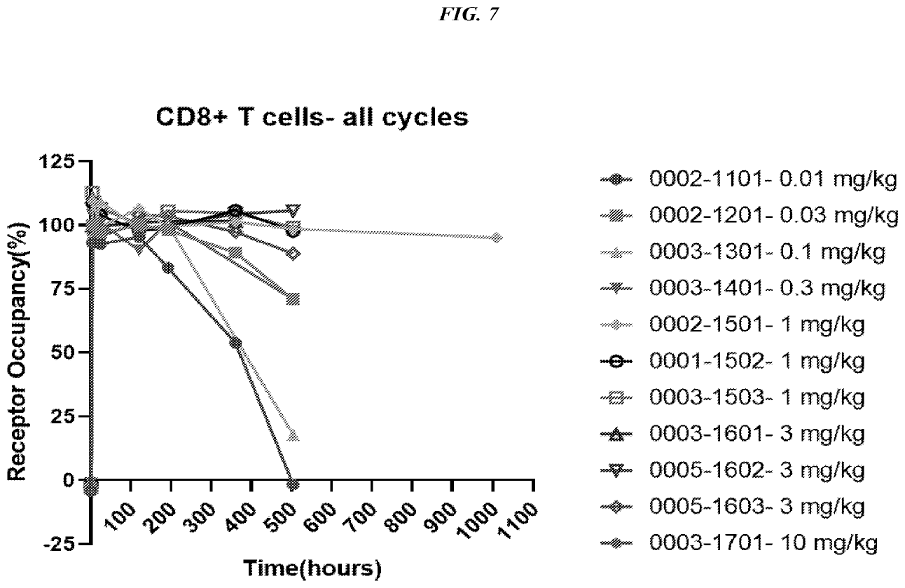

The formulated material was subjected to the stress and storage conditions in FIGS. 7A-7B.

### Formulation Study Procedural Steps

### Sampling Requirements

For each condition, a total of two vials were required for testing with one additional spare vial. One vial was required for LabChip (reduced and non-reduced), cIEF, concentration (A280 nm), the potency assay and SEC-HPLC analyses at CPS. The other vial was required for visual appearance and MFI analyses. Appropriate samples were removed at the indicated time points and frozen at <−60° C. until initiation of analysis, except samples for MFI and appearance testing. MFI and appearance testing which were performed immediately after pull.

The time zero (TO) samples of the 20 formulated samples were taken from the stock of vials stored at 2-8° C. and frozen at <−60° C. until analysis (or analyzed immediately in the case of appearance and MFI testing).

### Freeze/Thaw Study Storage at <−60° C.

For each cycle, three vials per formulation (total of 60 vials) were placed in storage at <−60° C. After a minimum of 16 hours, all three vials per formulation were pulled out of the <−60° C. storage condition (total of 60 vials) and allowed to warm to room temperature for 3-5 hours until thawed.

After being placed in the freezer for Cycle 3, the three vials were not thawed at room temperature until testing was ready to be initiated. Samples were then brought to room temperature prior to analysis.

Samples were Assayed as Described in this Example.

### Agitation

Three vials from each of the 20 formulations (total of 60 vials) were placed and fixed on a shaker rotating at ˜200 rpm at room temperature. Vials were agitated for no less than 24 hours and no more than 48 hours. All vials were stored frozen at <−60° C. until analysis, except MFI and appearance testing, which was performed immediately. Samples were equilibrated to room temperature prior to analysis.

Samples were assayed as described in this example.

### 2-8° C. Storage for 8 Weeks (Testing at 0, 2, 4 and 8 Weeks)

Twelve vials were taken from each of the 20 formulations (a total of 240 vials including TO vials) and stored at 2-8° C. Two vials at TO were analyzed immediately for MFI and appearance. All other samples were labeled with temperature and time point and stored frozen at <−60° C. until analysis. Samples were brought to room temperature prior to analysis.

At each subsequent time point, three vials were taken per formulation out of the 2-8° C. storage condition, labeled with the temperature and time point and stored frozen at <−60° C. until analysis except MFI and appearance testing, which was performed immediately. Samples were brought to room temperature prior to analysis.

Samples were assayed as described in this example.

### Ambient Storage (25° C.) for 4 Weeks (Testing at 2 and 4 Weeks)

Six vials were taken from each of the 20 formulations (total 120 vials) and stored at ambient temperature (25° C.).

At each time point, three vials were taken per formulation out of ambient storage, labeled with temperature and time point and frozen at <−60° C. until analysis except MFI and appearance testing, which was performed immediately. Samples were brought to room temperature prior to analysis.

Samples were assayed as described in this example.

### 40° C. Storage for 2 Weeks (Testing at 1 and 2 Weeks)

Six vials were taken from each of the 20 formulations (total 120 vials) and stored at 40° C.

At each time point, three vials were taken per formulation out of 40° C. storage, labeled with temperature and time point and store frozen at <−60° C. until analysis except MFI appearance testing, which was performed immediately. Samples were brought to room temperature prior to analysis.

Samples were assayed as described in this example.

### Testing Plan and Schedule

For each formulation condition, sample and test were performed.

### Results

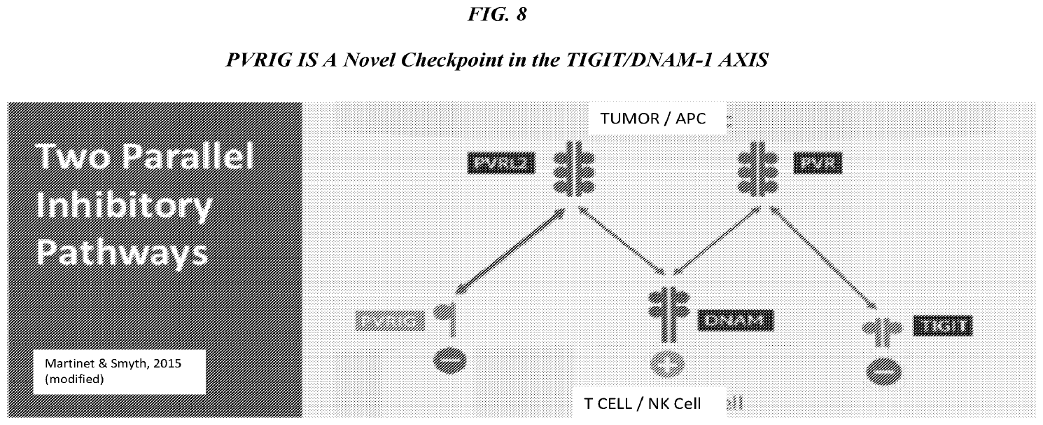

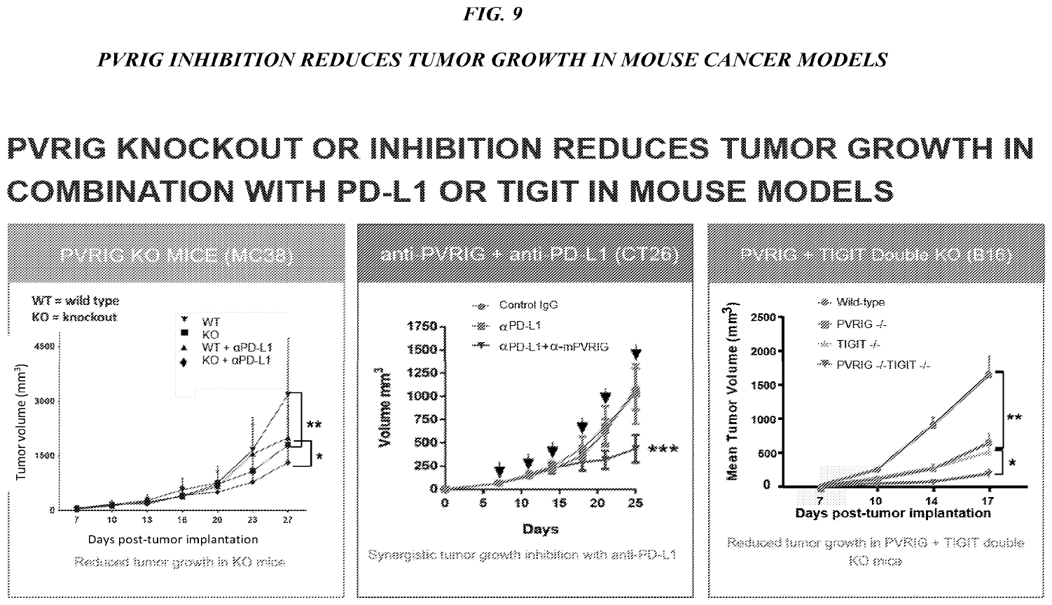

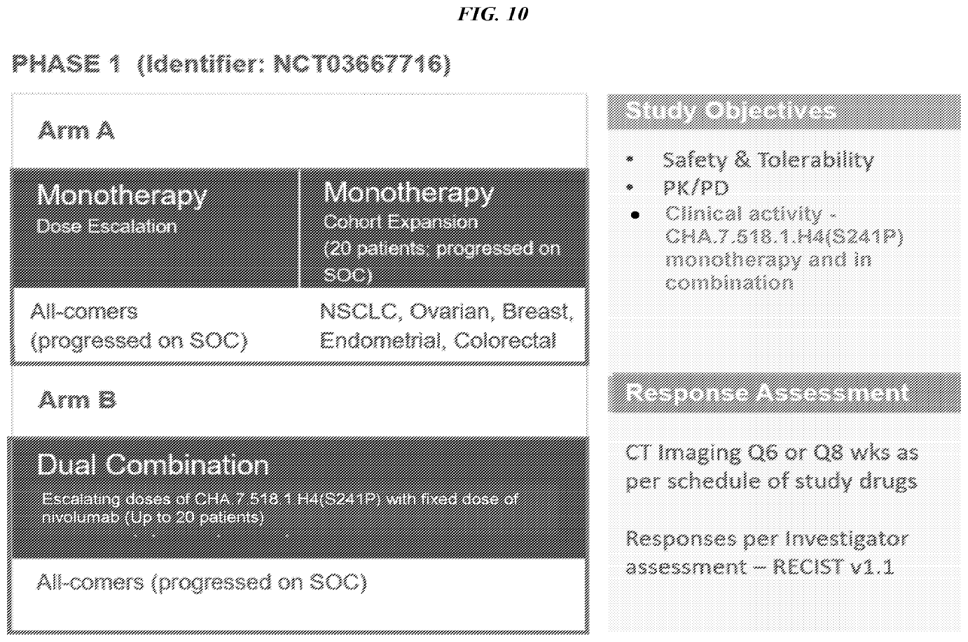

All data from each formulation and time point was provided throughout the study and are presented in FIGS. 8-79). The results are discussed in this example. Each formulation was evaluated and compared to the conditions studied.

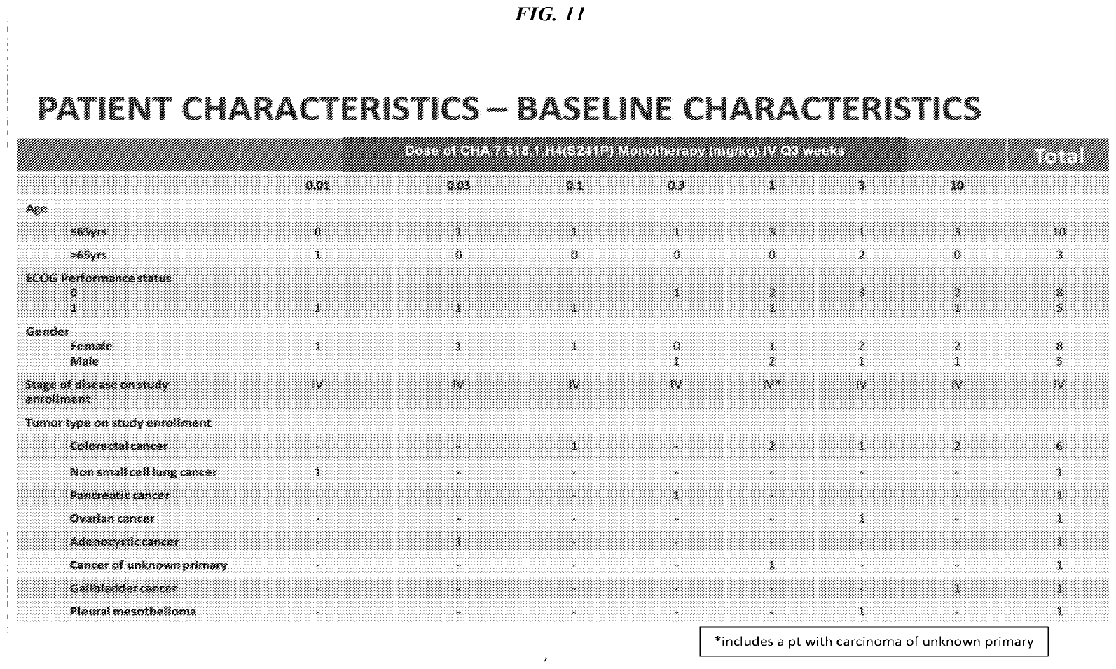

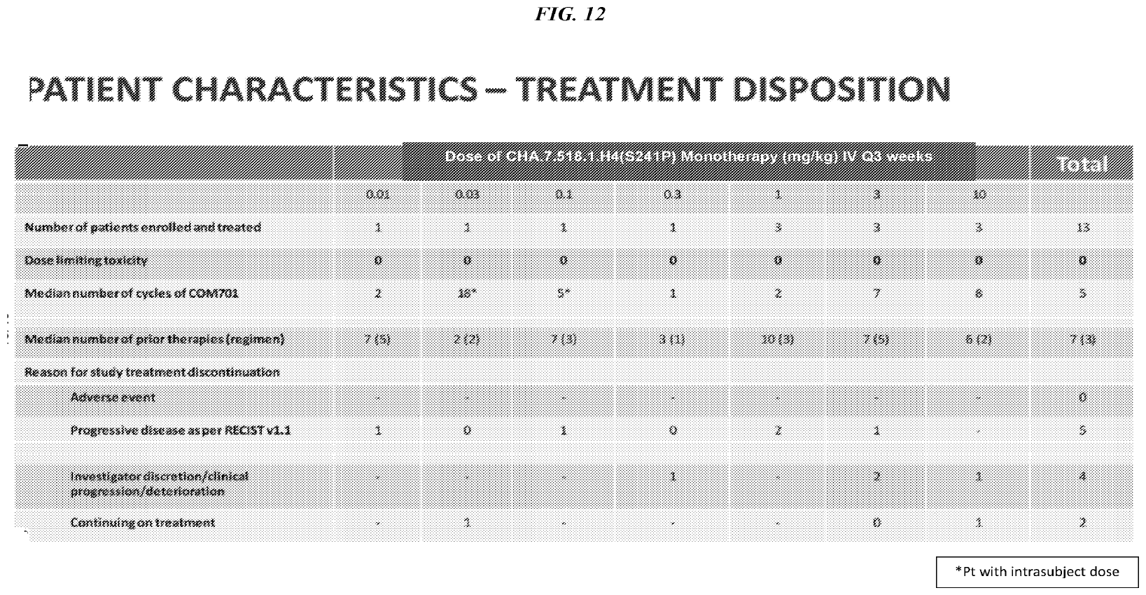

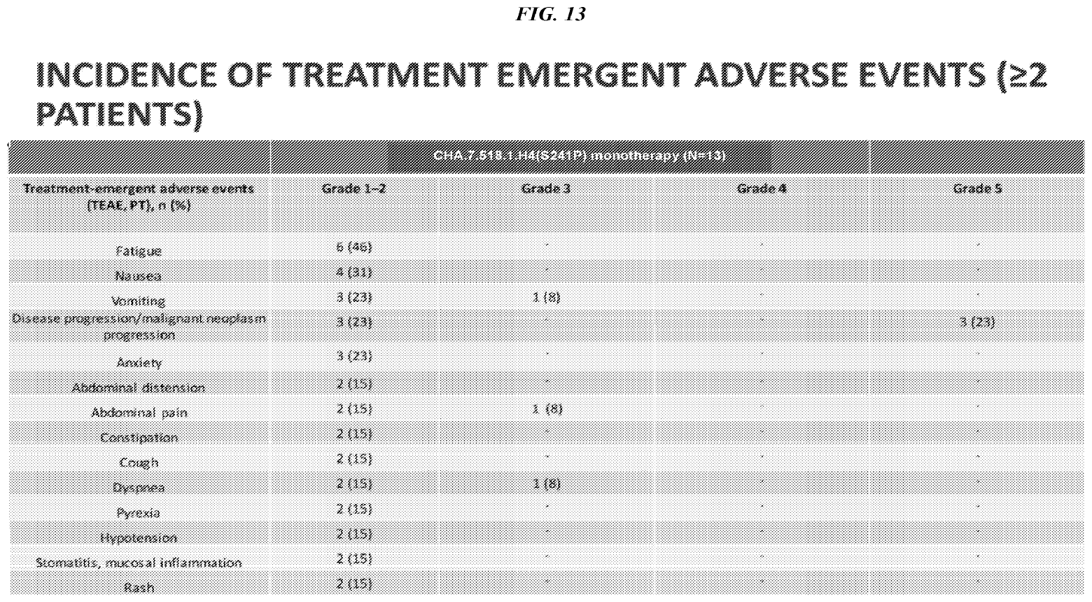

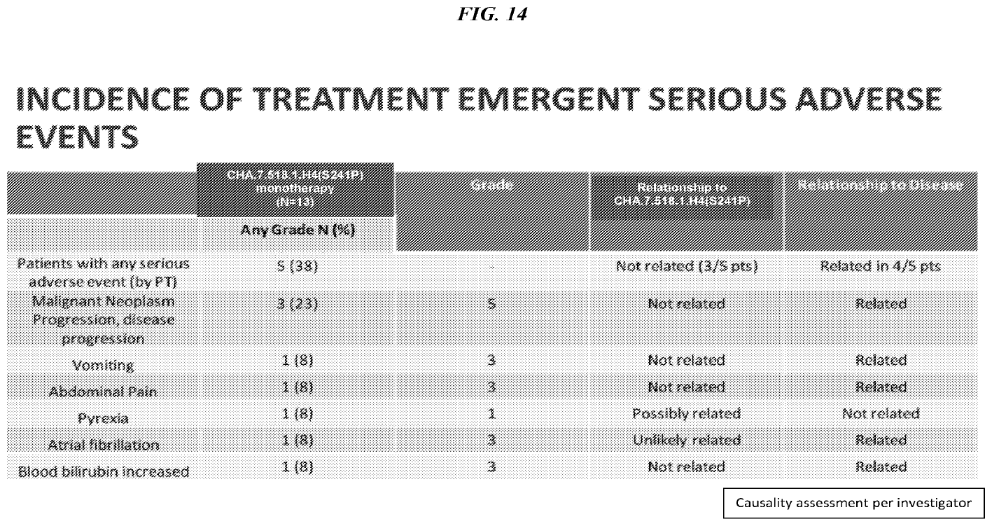

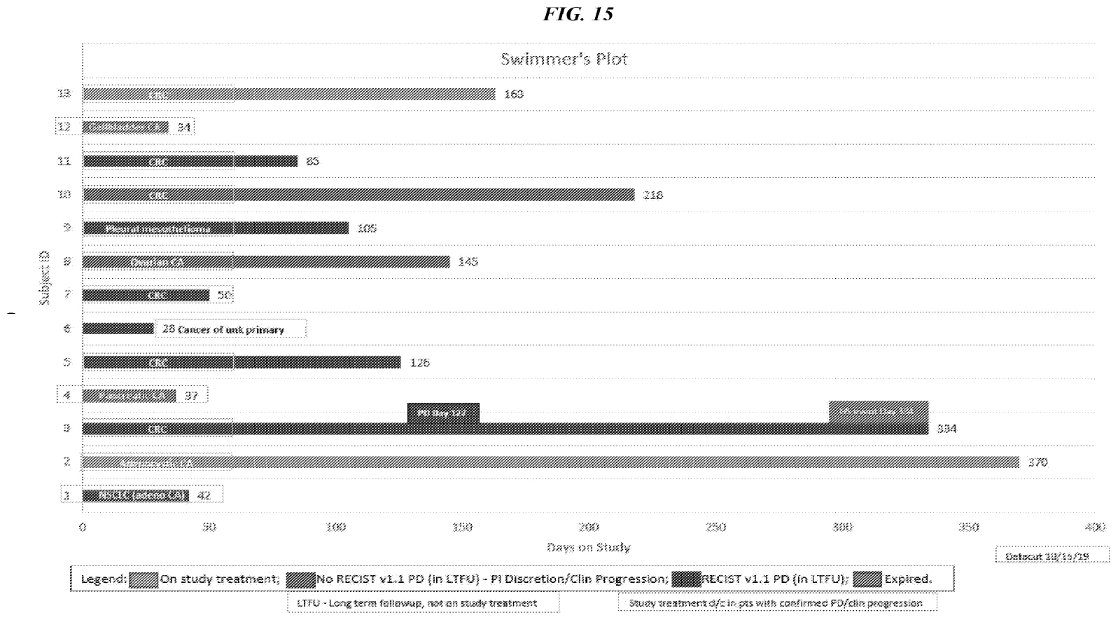

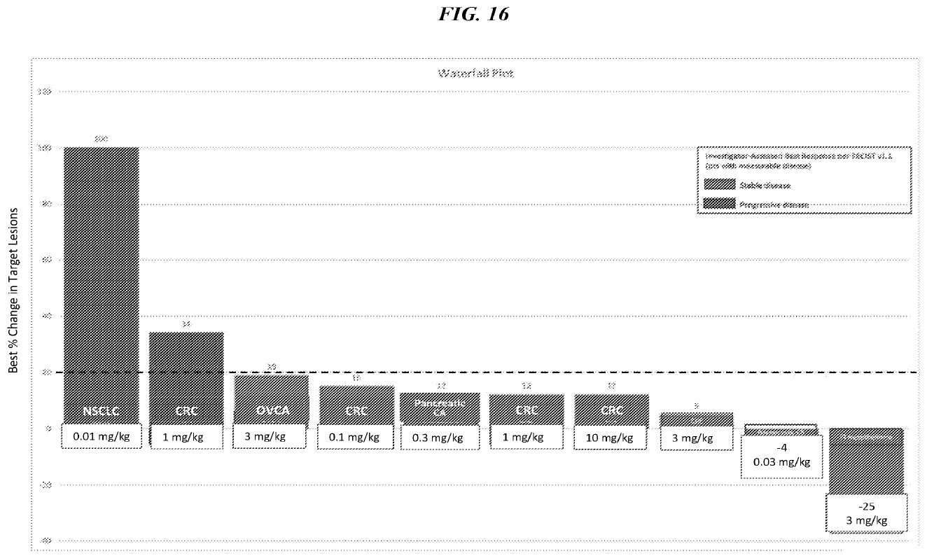

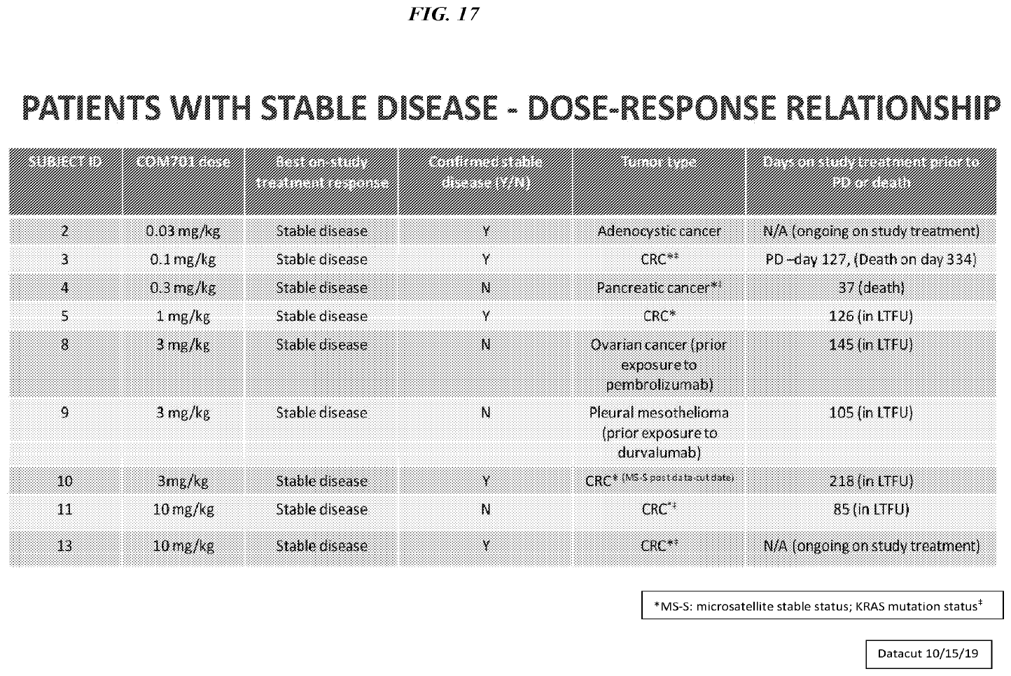

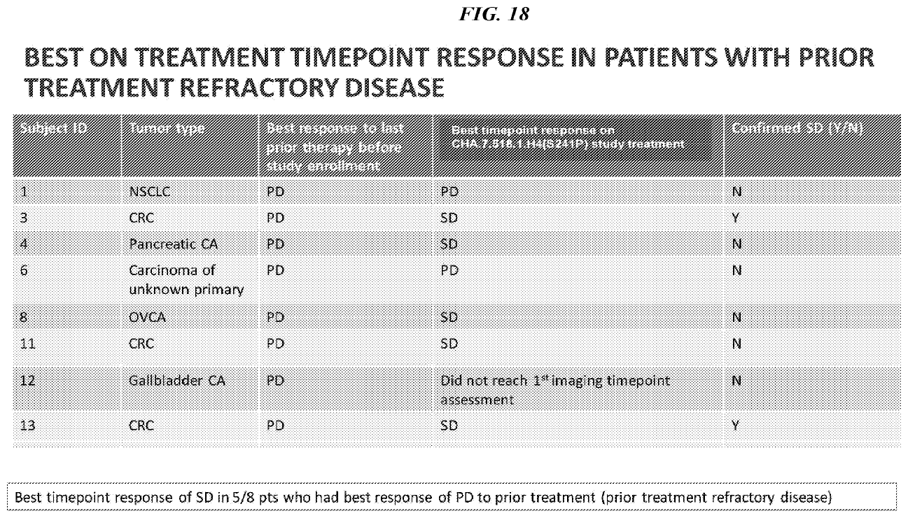

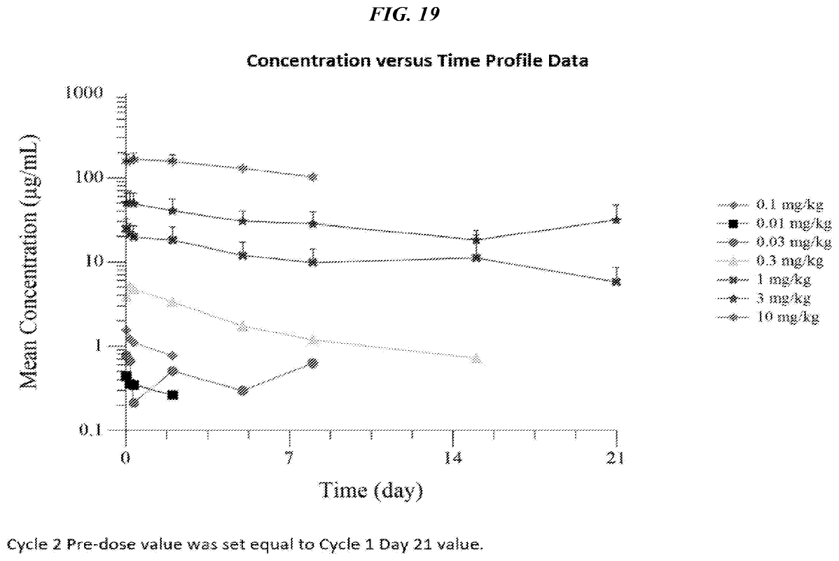

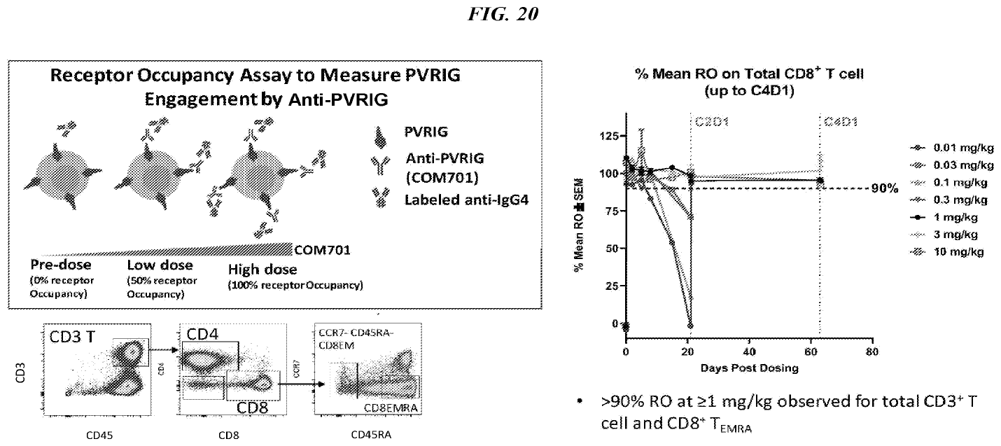

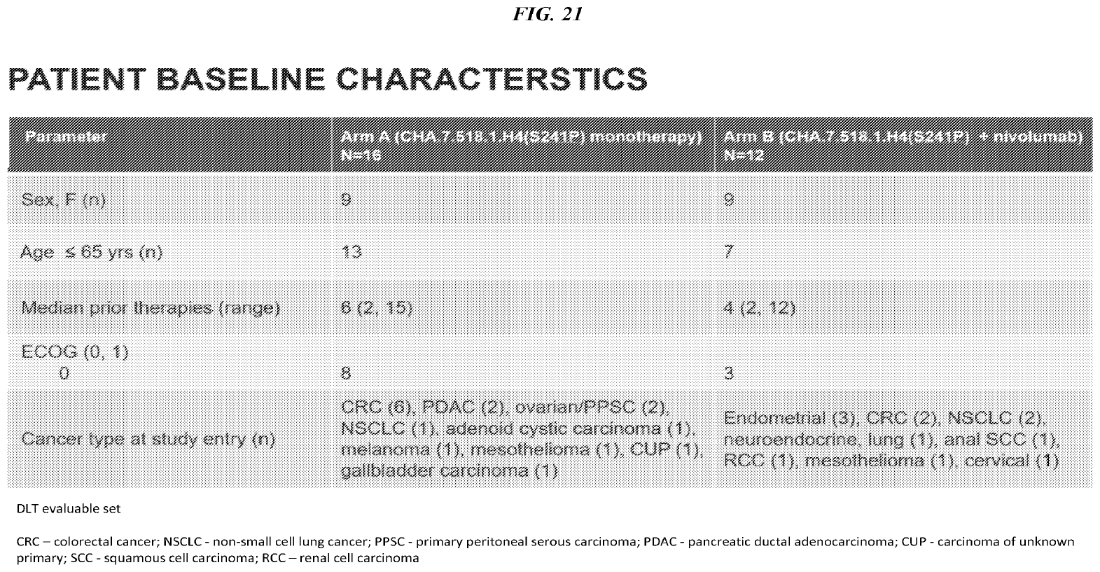

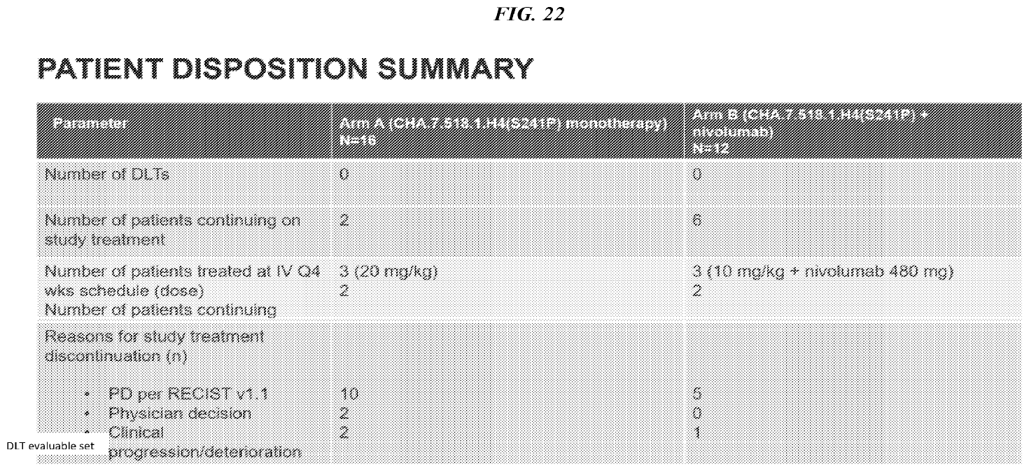

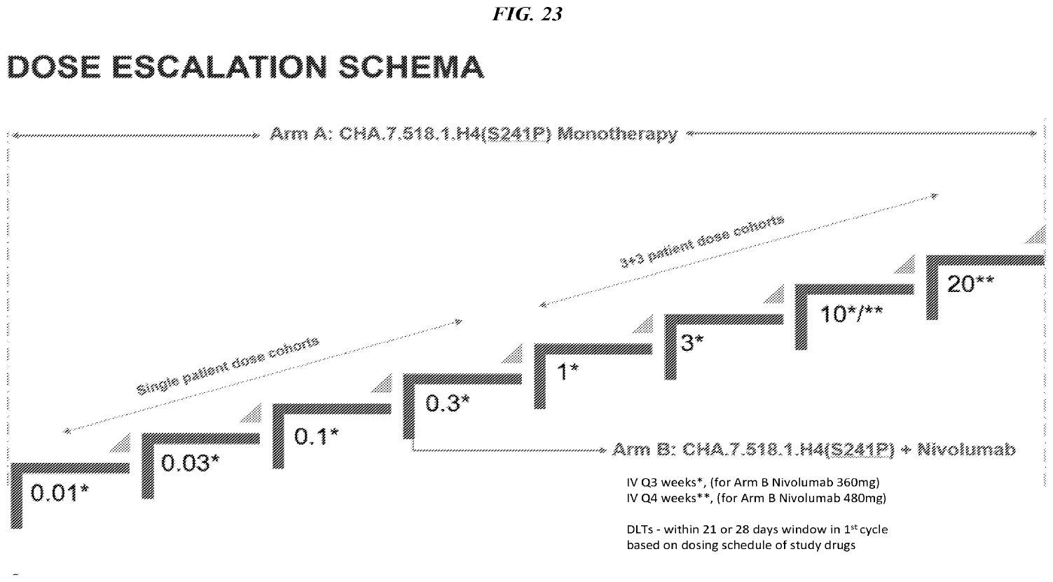

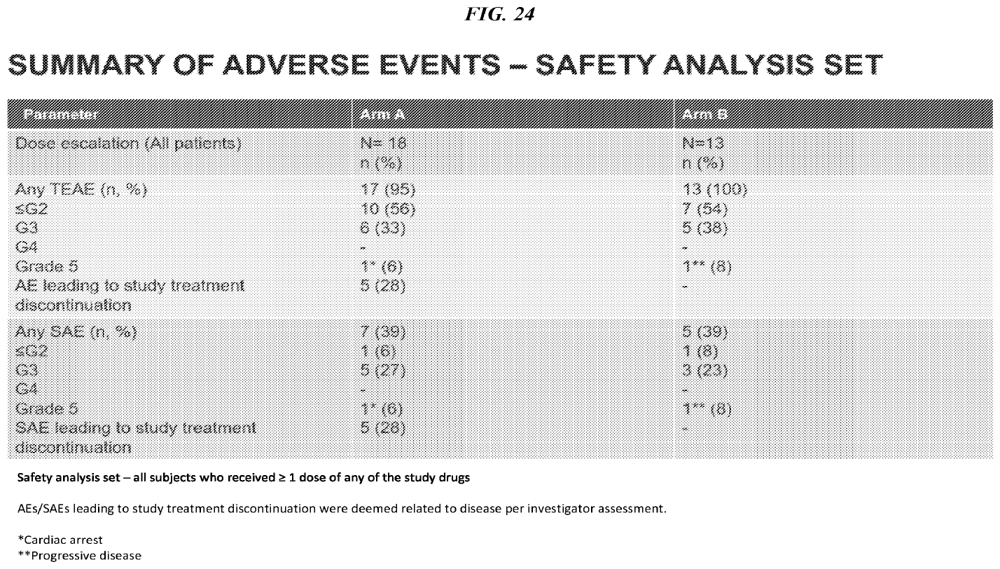

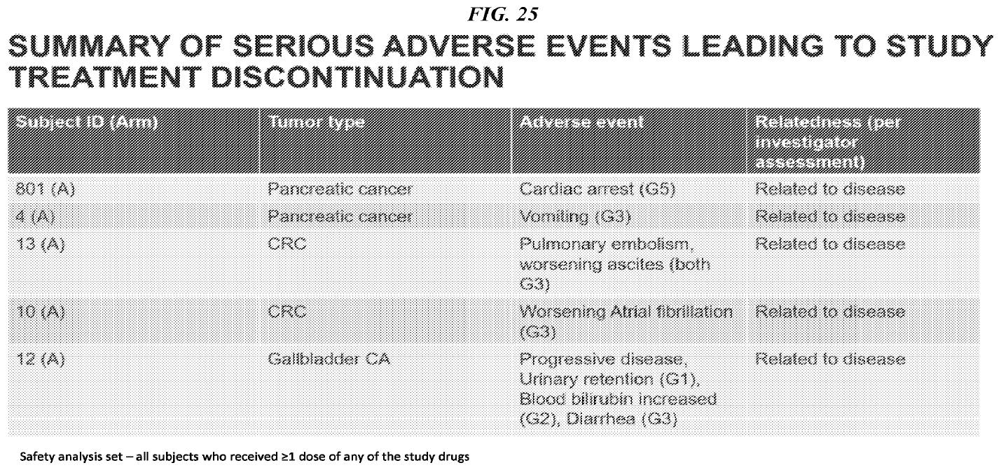

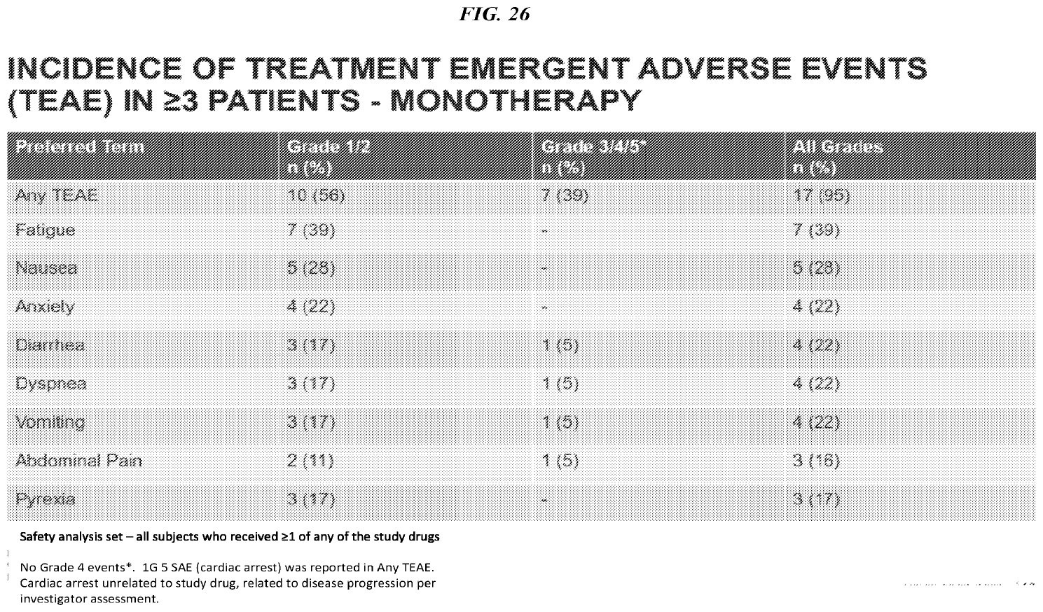

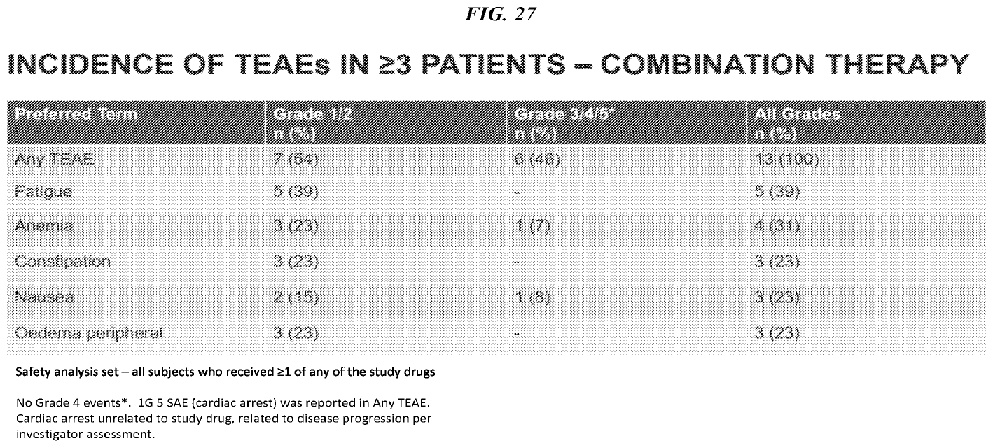

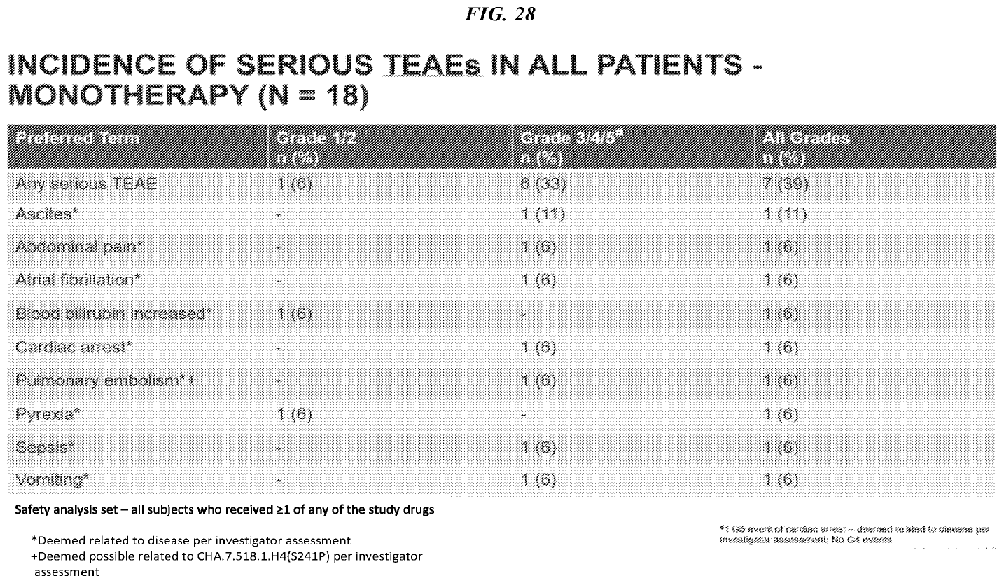

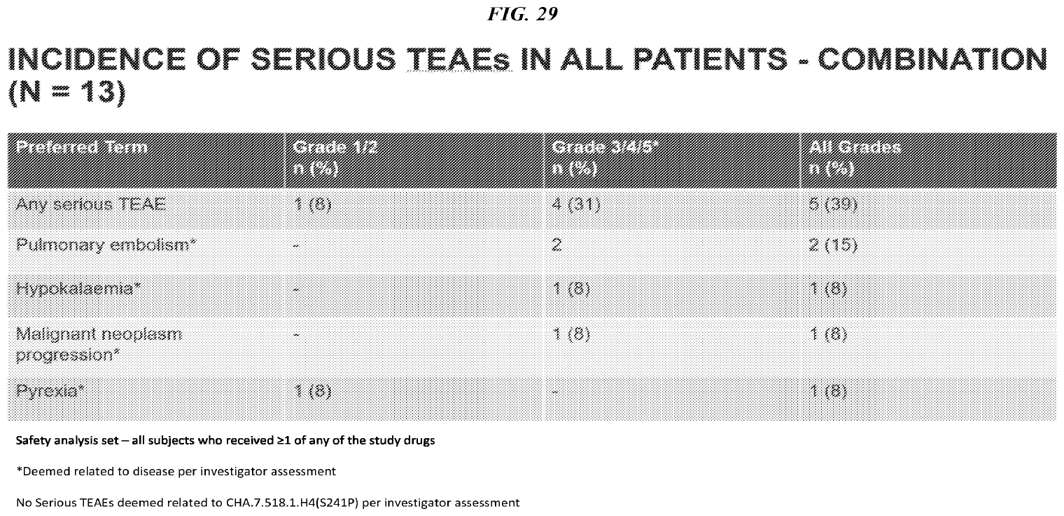

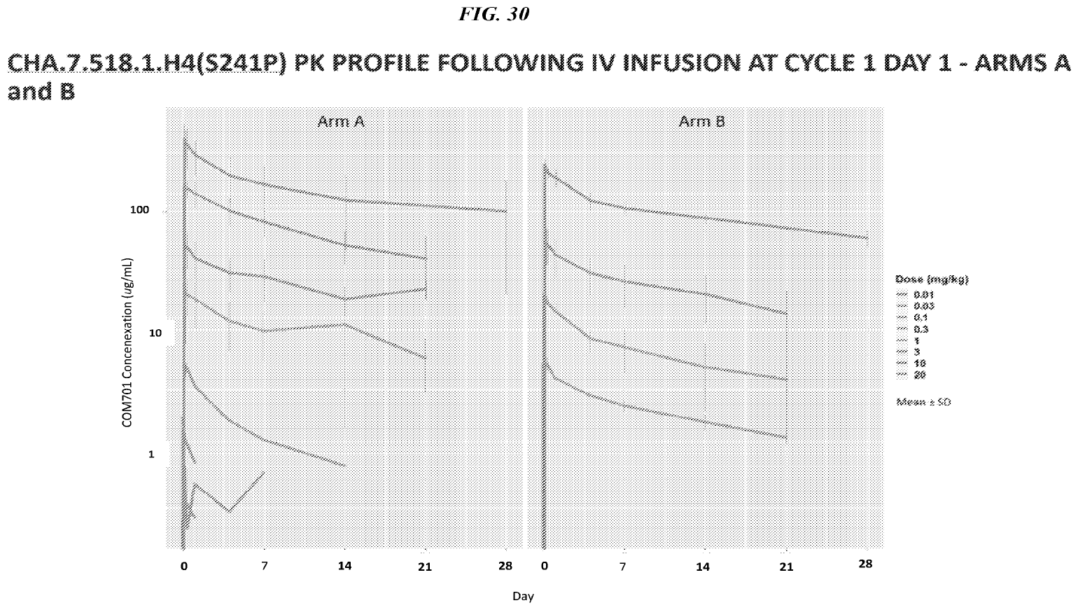

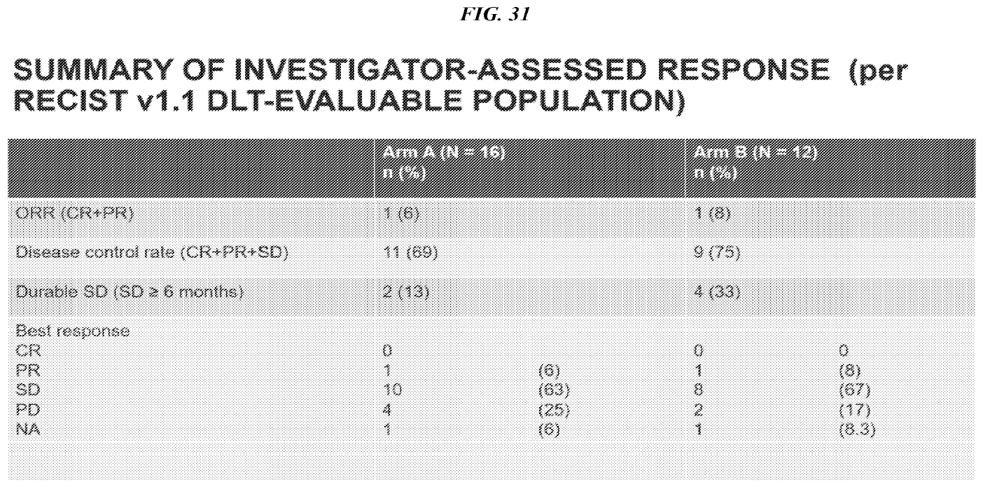

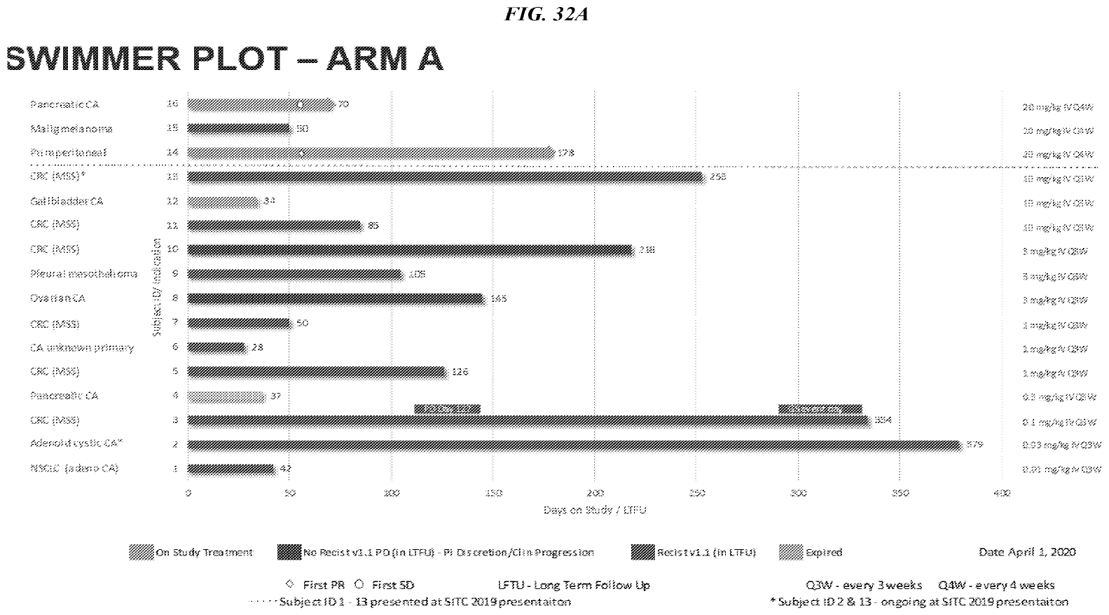

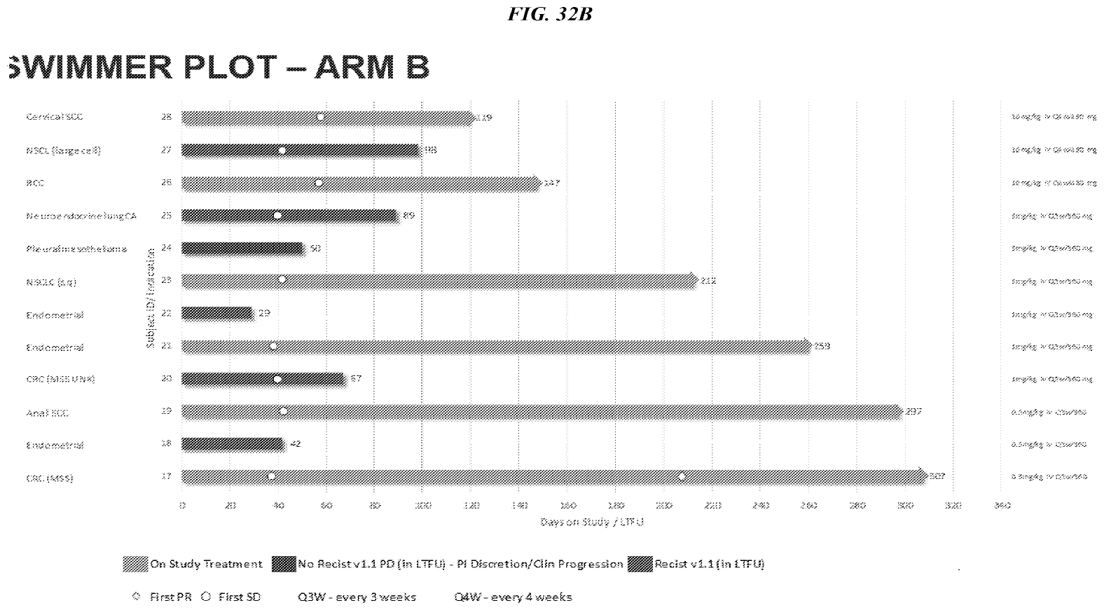

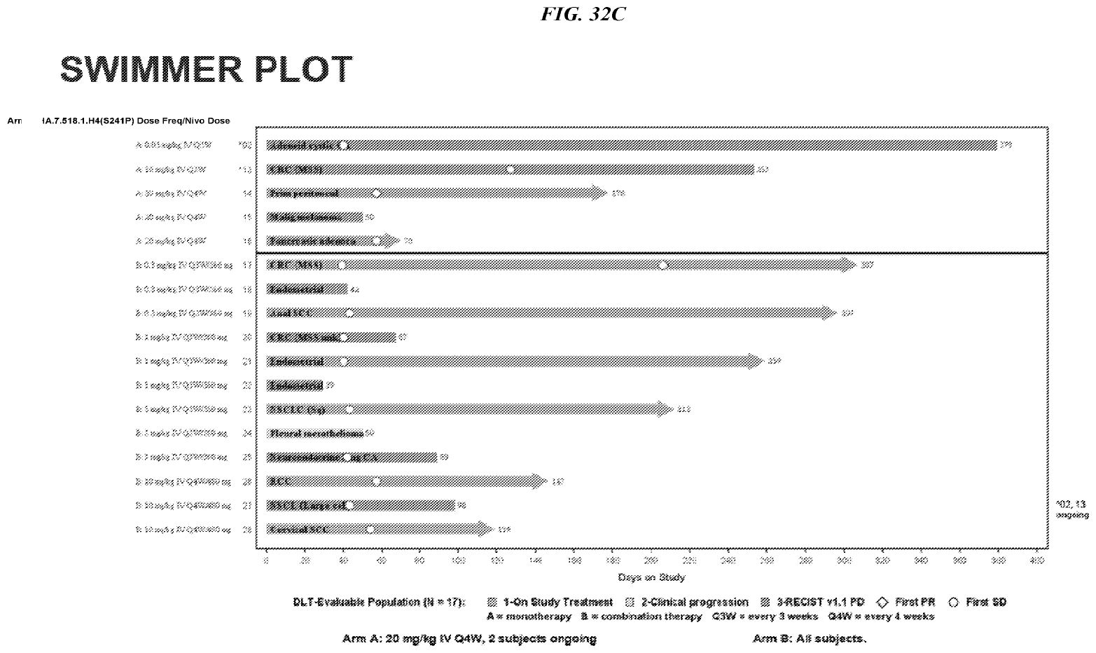

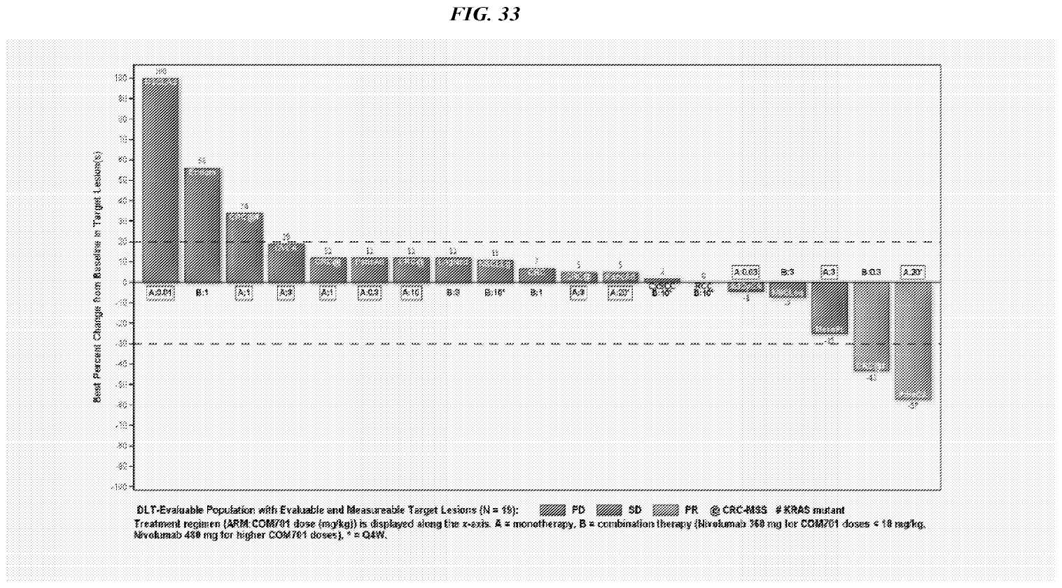

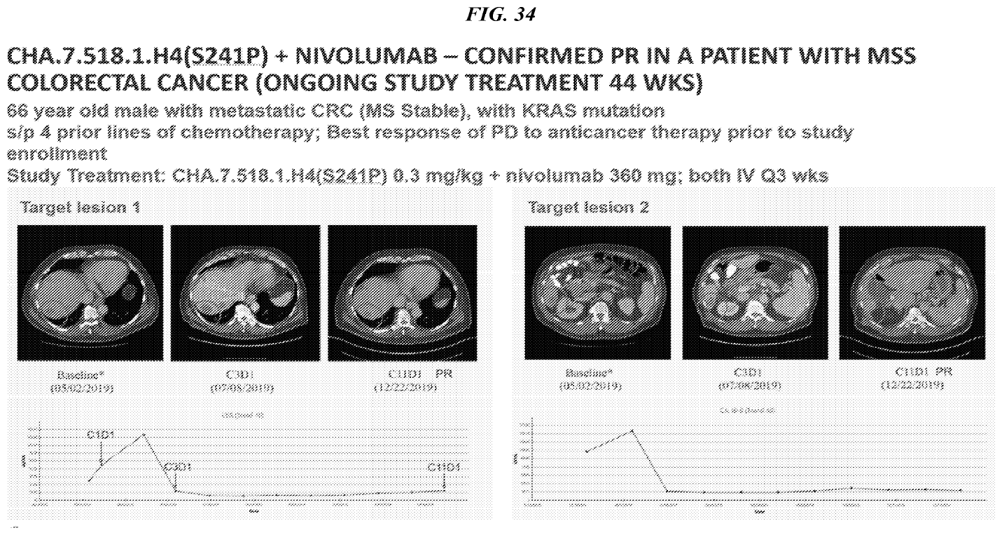

The figures provide graphical representations of the critical assay results that were compiled and analyzed. The critical assays analyzed to determine an appropriate formulation were SEC, cIEF and MFI. SEC high molecular and low molecular weight species were monitored throughout the study (FIGS. 11, 18, 25, 33, 40, 47, 54, 61, 68, and 75). cIEF results were obtained throughout the study (FIGS. 12, 19, 26, 34, 41, 48, 55, 62, 69, and 76). Finally, MFI particles/mL throughout the various sizes were monitored (FIGS. 13, 20, 27, 35, 42, 49, 56, 63, 70, and 77).

### DISCUSSION

### A280 and Appearance Analysis

A280 by SoloVPE and appearance testing showed no significant changes across time points and formulations and were not used to determine a final formulation.

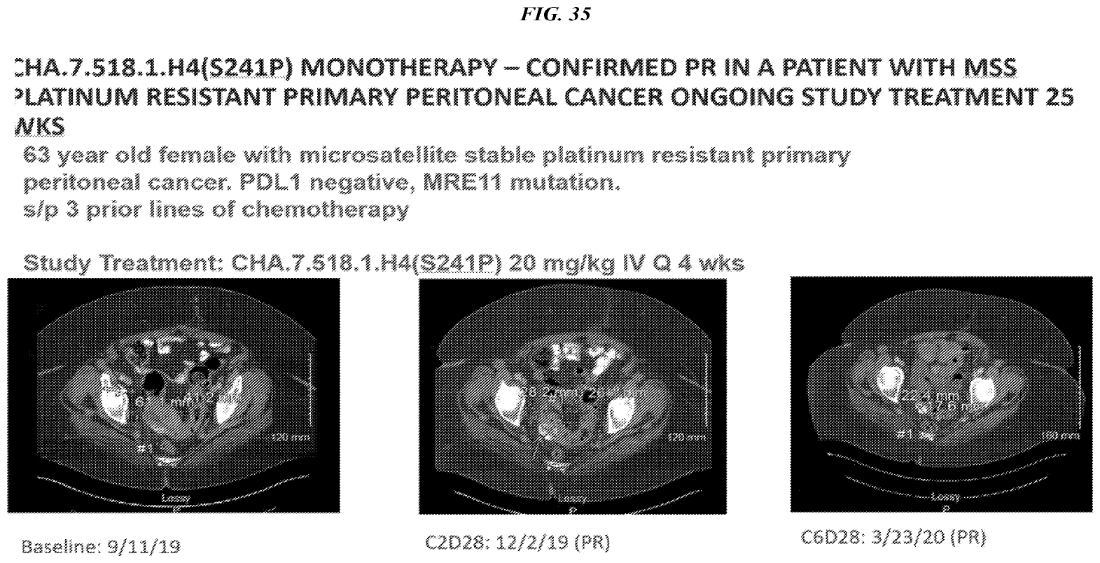

During the freeze/thaw analysis, the SoloVPE yielded varying concentrations across all different formulations beyond the instrument specifications. The spare vials were pulled and the analysis repeated. However, the analysis still showed varying results. Therefore, the same samples were repeated using 320 nm correction for light scattering. The A280 results showed much less variability (FIG. 35). The freeze/thaw data shows that 320 nm correction may be necessary for this product after repeated freeze/thaw. Other conditions did not yield this variability. Since this product will undergo a prolonged stability study, it will be required that this product use a 320 nm correction when the SoloVPE is used for concentration determination.

### Binding Assay Analysis

Evaluation of the binding assay was performed, however the binding assay showed no significant changes in activity across conditions or formulations. The changes that were observed were within the method variability. Therefore, this method shows the molecule is stable in terms of binding activity. This method was not a critical assay in making a formulation decision.

### LabChip Analysis

Evaluation of the LabChip data showed that IgG purity and HC+LC percentages were fairly stable across time points and conditions. IgG purity percentages ranged from 96 to 97% and HC+LC percentages ranged from 98 to 100%. Since there was no significant change in the results across time points, this method was not used to determine a formulation.

cIEF Analysis

Upon generation of the 40° C. 1 week results from the cIEF analysis, it was found that an additional minor acidic species was present in Formulations A, B9, and B10. This led to the removal of these formulations from recommendation at this point in the study. However, following this time point, all accelerated conditions and the 2-8° C. 4 week time point and longer showed the presence of this minor species in varying quantities. Therefore, when deciding an appropriate formulation, the minor species was monitored for stability throughout the accelerated conditions.

### MFI Analysis

Protein can form sub-visible particles in response to stressed conditions, such as heat, freeze/thaw cycles, and agitation. An optimal formulation is capable of stabilizing the protein against these stressed conditions and protecting against the formation of particles. MFI was used to evaluate particle counts at different size ranges (<2 μm, <5 μm, <10 μm, and <25 μm) in different formulations under stressed conditions. The MFI data was evaluated to choose an appropriate formulation based on generation of the lowest amount of particles/mL for all sizes of particles across all time points, conditions, and formulations.

### SEC Analysis

The SEC data showed HMW throughout all time points and conditions; however, it remained stable at about 1%. LMW was present in accelerated conditions and 2-8° C. 8 week time point. Within the 40° C. condition, the LMW did increase from about 1% to 3% from Week 1 to Week 2. This species will be monitored throughout the program and later should be identified through further characterization analysis. When deciding an appropriate formulation, the LMW and HMW were evaluated for stability across the time points and conditions.

### Conclusion

With this data, it was determined that the buffer designated as B4 (25 mM histidine, 60 mM NaCl, 100 mM L-Arginine, 0.01% PS 80, pH 6.5) would be the final formulation. This formulation had consistent SEC results with low HMW and LMW. In addition, the MFI data showed lower amounts of particles/mL for all particle sizes. LabChip data showed that the IgG purity and HC+LC percentages were stable in formulation B4 when compared to TO. Therefore, the toxicology batch was formulated in this buffer.

### Example 2: PVRIG Antibody Formulation

### Description and Composition of Solution for Infusion

The formulation is provided as a sterile preservative free liquid dosage form at a concentration of 20 mg/mL in a 10R Type I clear borosilicate glass vial equipped with a gray bromobutyl rubber stopper and aluminum flip cap crimp. The vials are filled to a target volume of 10 mL. The formulation is stored and shipped frozen at −20 C. Prior to use, the vials are thawed at ambient temperature, mixed by gentle swirling. For administration to patients, the formulation is diluted with 0.9% sodium chloride.

### Container Closure System

A single container closure system exists for the formulation and is comprised of a 10R Type I clear borosilicate glass vial, a 20 mm bromobutyl rubber stopper and a 20 mm aluminum flip cap crimp.

The formulation was produced by thawing and pooling the Drug Substance, followed by 0.22 μm sterile filtration and filling into sterile 10R glass vials at Vetter.

The formulation components and quantitative composition of the drug product on a nominal amount per vial unit (10 mL) is presented in the Table 1 below (see, also B4 in FIGS. 6-78).

TABLE 1
Composition

| | | | Nominal |
|---|---|---|---|
| | | | amount |
| | | Presentation | [mg/vial] |
| Component | Function | 10R Glass Vial | V = 10.0 mL |
| CHA.7.518.1.H4(S241P) | Active | 20 mg/ml | 200 |
| mAb | Ingredient | in liquid | |
| | | dosage form | |
| 25 mM | Buffer | 25 mM in liquid | 39 |
| Histidine | Component - | formulation | |
| | pH Stabiliza- | | |
| | tion | | |
| 60 mM NaCl | Buffer | 60 mM in liquid | 35 |
| | Component- | formulation | |
| | Ionic | | |
| | Strength | | |
| | Control | | |
| 100 mM L- | Formulation | 100 mM in | 174 |
| Arginine | Excipient- | liquid | |
| | Stabilizer | formulation | |
| Polysorbate 80 | Formulation | 0.01% w/v in | 1 |
| | Excipient | liquid | |
| | Surfactant | formulation | |

A sufficient volume is filled into vials based on the net fill weight to ensure a withdrawable volume of 10 mL.

### Example 3: A Phase 1 Study Evaluating an Anti-PVRIG Antibody in Patients with Advanced Solid Tumors

### Background

There is a high unmet medical need for the treatment (tx) of patients (pt) who are refractory to or relapse following treatment with checkpoint inhibitors. Newer checkpoint therapies with novel mechanisms of action that can activate T cells and demonstrate antitumor activity in this pre-treatment patient population are urgently needed. CHA.7.518.1.H4(S241P) (heavy chain: SEQ ID NO:8; light chain: SEQ ID NO:13) is a novel first-in-class humanized IgG4 monoclonal antibody that binds with high affinity to PVRIG (poliovirus receptor related immunoglobulin domain containing) blocking its interaction with its ligand, PVRL2. Both PVRIG and PVRL2 are part of the DNAM axis as are TIGIT and PD1. Inhibition of PVRIG leads to enhanced activation of T and NK cells, and PVRIG results in tumor growth inhibition in mouse tumor models. We hypothesize that CHA.7.518.1.H4(S241P) (heavy chain: SEQ ID NO:8; light chain: SEQ ID NO:13) will demonstrate antitumor activity in pts who are checkpoint inhibitor pre-treatment.

### Methods:

This example describes an ongoing open-label first-in-human phase 1 study in patients with advanced solid tumors. The initial part of this study (Arm A) will evaluate escalating doses of CHA.7.518.1.H4(S241P) (heavy chain: SEQ ID NO:8; light chain: SEQ ID NO:13) monotherapy IV Q3 weekly with single pt cohorts for the initial 4 and then 3+3 design. Key Inclusion Criteria: Age ≥18 yrs, histologically confirmed locally advanced/metastatic solid malignancy and has exhausted available standard therapy, ECOG 0-1, prior anti-PD-1, anti-PD-L1, anti-CTLA-4, OX-40, CD137 permissible.

Key Exclusion Criteria: Active autoimmune disease requiring systemic therapy in the last 2 years, symptomatic interstitial or inflammatory lung disease, untx or symptomatic central nervous system metastases. Primary objectives are safety and tolerability of CHA.7.518.1.H4(S241P) (heavy chain: SEQ ID NO:8; light chain: SEQ ID NO:13) as measured by the incidence of adverse events (AEs) and dose-limiting toxicities (21-day DLT window), pharmacokinetics of CHA.7.518.1.H4(S241P) (heavy chain: SEQ ID NO:8; light chain: SEQ ID NO:13), and to identify the maximum tolerated dose and/or the recommended dose for expansion. Secondary objectives are to characterize the immunogenicity and preliminary antitumor activity of CHA.7.518.1.H4(S241P) (heavy chain: SEQ ID NO:8; light chain: SEQ ID NO:13).

Statistical Considerations: AEs graded as per CTCAE v4.03, responses as per RECIST v1.1. The analyses of all study objectives will be descriptive and hypothesis generating. No DLTs have been observed in the single pt cohorts. Assessment of pts enrolled into cohort 5 is ongoing at the time of this submission.

### Example 4: Phase 1 Study Evaluating an Anti-PVRIG Monotherapy and in Combination with Nivolumab in Patients with Advanced Solid Tumors

### Background

CHA.7.518.1.H4(S241P) is a novel 1st-in class checkpoint inhibitor of poliovirus receptor related immunoglobulin domain (PVRIG). It inhibits the binding of PVRIG with its ligand, PVRL2. Nivolumab an anti-PD-1 is approved in pts with advanced malignancies (Nvolumab package insert. http://packageinserts.bms.com/pi/pi_opdivo.pdf Accessed Jul. 22, 2019). It has been demonstrated that the DNAM signaling axis consisting of PVRL2, TIGIT and DNAM plays a role in regulating the activity of T/NK-cells. PD-1 inhibitors also play an important role in this axis by modulating DNAM activation. In preclinical experiments it has been demonstrated that blocking PVRIG alone and in combination with PD-1 inhibition leads to activation of T cells in the tumor microenvironment thereby generating an anti-tumor immune response and tumor growth inhibition. There is a high unmet medical need for novel immune checkpoint inhibitors (ICI) as monotherapy in pts who relapse after treatment with approved ICI, and in combination with approved ICI to deepen clinical responses. While not being bound by theory, it is hypothesized that CHA.7.518.1.H4(S241P) will be safe and tolerable and demonstrate preliminary antitumor activity as monotherapy and in combination with nivolumab in pts with R/R solid tumors. It has previously been reported that no DLTs were reported up to dose level 6 with CHA.7.518.1.H4(S241P) monotherapy (A phase I study evaluating CHA.7.518.1.H4(S241P) in patients with advanced solid tumors. J Clin Oncol 37, 2019 (suppl; abstr TPS2657)).

### Methods:

Ongoing P1 dose-escalation study with single pt cohorts and 3+3 study design of CHA.7.518.1.H4(S241P) as monotherapy IV Q3 weeks and in combination with nivolumab 360 mg IV Q3 weeks. Key Inclusion Criteria: Age ≥18 yrs, histologically confirmed advanced solid tumor and has exhausted all available standard therapy, ECOG 0-1, prior anti-PD-1, anti-PD-L1, anti-CTLA-4, OX-40, CD137 permissible. Key Exclusion Criteria: Active autoimmune disease requiring systemic therapy in the last 2 years, symptomatic interstitial or inflammatory lung disease, untx or symptomatic CNS metastases. Primary objectives: safety and tolerability of CHA.7.518.1.H4(S241P) monotherapy and in combination with nivolumab measured by the incidence of AEs and DLTs (21-day window), pharmacokinetics of CHA.7.518.1.H4(S241P), and to identify the maximum tolerated dose and/or the recommended dose for expansion as monotherapy/in combination with nivolumab. Secondary objectives: characterize the immunogenicity and preliminary antitumor activity of CHA.7.518.1.H4(S241P) in combination with nivolumab. Statistical Considerations: AEs as per CTCAE v4.03, responses as per RECIST v1.1. Analyses of study objectives are descriptive and hypothesis generating.

### Results:

At this submission date no DLTs have been observed up to dose level 7 of CHA.7.518.1.H4(S241P) monotherapy and dose level 1 of CHA.7.518.1.H4(S241P) in combination with nivolumab.

### Conclusion:

Assessment of safety and tolerability is ongoing for all pts. Updated results will be analyzed throughout the clinical trial.

### Example 5: Phase 1 Study of the Safety, Tolerability and Preliminary Anti-Tumor Activity of CHA.7.518.1.H4(S241P) Monotherapy in Patients with Advanced Solid Tumors

### Background

CHA.7.518.1.H4(S241P) is a novel first-in-class immune checkpoint inhibitor (ICI) of poliovirus receptor related immunoglobulin domain (PVRIG) [1]. It inhibits the binding of PVRIG with its ligand, PVRL2. PVRIG is a member of the DNAM/TIGIT signaling axis regulating the activity of T/NK-cells. In preclinical experiments we have demonstrated that PVRIG inhibition alone and in combination with anti-PD-1 and/or TIGIT blockers leads to activation of T cells in the tumor microenvironment generating an anti-tumor immune response and tumor growth inhibition [1]. Although ICI revolutionized cancer treatment, there is an urgent need to develop treatments for patients who are refractory or relapse after treatment with ICI. The study was designed to show CHA.7.518.1.H4(S241P) to be safe, tolerable and demonstrate preliminary anti-tumor activity.

### Methods:

A phase 1a, dose-escalation of CHA.7.518.1.H4(S241P) monotherapy utilizing a hybrid accelerated and 3+3 study design was conducted to determine safety, tolerability, to assess the pharmacokinetics (PK), pharmacodynamics, to determine the recommended phase 2 dose and to evaluate preliminary anti-tumor activity of CHA.7.518.1.H4(S241P). Patients with performance status ECOG 0-1 and advanced solid tumors who failed standard of care treatments were eligible for inclusion. Prior ICIs were permissible. CHA.7.518.1.H4(S241P) 0.01, 0.03, 0.1, 0.3, 1, 3 and 10 mg/kg IV every 3 weeks were administered until progression, intolerable toxicity or investigator or patient discretion. Adverse events were reported per CTCAE v4.03 and anti-tumor activity was evaluated using RECIST v1.1. Dose-limiting toxicities (DLTs) were evaluated within a 21-day window.

### Results:

A total of 13 patients were enrolled and treated during dose escalation of CHA.7.518.1.H4(S241P), including 6 patients with metastatic colorectal cancer (CRC), 5 with microsatellite stable status (MSS) and 1 unknown. Patients were heavily pretreated with a median of 7 prior anticancer therapies (range 2-15). No DLTs have been reported up to 10 mg/kg CHA.7.518.1.H4(S241P) dose level. The most frequent toxicities were fatigue (8%), abdominal pain (6%). Likely immune-related adverse events: elevated TSH and rash were observed in 2 patients. Overall 7/13 patients (54%) maintained best response of stable disease (SD)≥12 weeks (13.6-43 weeks), including 5/6 (83%) of patients with CRC. Five patients continue on study treatment. Peripheral PVRIG receptor occupancy (>90%) was demonstrated at ≥1 mg/kg dose of CHA.7.518.1.H4(S241P) and PK profile supports IV Q3 weekly dosing.

Conclusion:

CHA.7.518.1.H4(S241P) monotherapy demonstrated an acceptable safety and tolerability profile with preliminary anti-tumor activity in a patient population that had received multiple prior anti-cancer therapies.

### REFERENCE

1. Spencer L, Ofer L et al, Discovery of COM701, a therapeutic antibody targeting the novel immune checkpoint PVRIG, for the treatment of cancer. J Clin Oncol. 2017; (suppl; abstr 3074).

### Example 6: Data from Ongoing Phase 1 Trial of CHA.7.518.1.H4(S241P) in Patients with Advanced Solid Tumors

CHA.7.518.1.H4(S241P) was well tolerated with no dose-limiting toxicities observed.

Initial signals of anti-tumor activity observed in heavily pretreated patient population in the dose escalation arm of the study.

Preliminary results from the ongoing Phase 1 dose escalation study of CHA.7.518.1.H4(S241P), its first-in-class anti-PVRIG antibody, in patients with advanced solid tumors. CHA.7.518.1.H4(S241P) was well tolerated with no dose-limiting toxicities. Furthermore, CHA.7.518.1.H4(S241P) demonstrated initial signals of anti-tumor activity in the heavily pretreated patient population enrolled on the study.

The emerging safety profile and initial anti-tumor activity of CHA.7.518.1.H4(S241P) was encouraging. The primary objective of this portion of the trial was to test the safety and tolerability of CHA.7.518.1.H4(S241P) in an all-comers population, and early signals of anti-tumor activity in hard to treat patients, including patients with microsatellite stable colorectal cancer (MSS-CRC). We look forward to initiating our biomarker driven CHA.7.518.1.H4(S241P) monotherapy expansion cohort in patients with ovarian, endometrial, breast and lung cancers. CHA.7.518.1.H4(S241P) can expand the checkpoint inhibitors landscape in these indications, which we chose based on our understanding of the PVRIG biological pathway.

Expanding the reach of cancer immunotherapy drugs to broader patient populations is an urgent need given the number of patients with advanced cancer who are non-responsive or refractory to currently available therapies. The initial signals of anti-tumor activity of CHA.7.518.1.H4(S241P) are encouraging, particularly given the heavily pretreated all-comer patient population, with majority of patients refractory to previous therapy. Attend in dose-response relationship in this difficult to treat patient population was observed and furthermore, an encouraging signal of anti-tumor activity in five out of six patients with MSS colorectal, a challenging indication, typically not responsive to current immune checkpoint blockers.

The reported data are from the monotherapy arm of the ongoing, Phase 1, open label, dose escalation study and include the first 6 cohorts (n=13) at dose levels of 0.01, 0.03, 0.1, 0.3, 1, 3, and 10 mg/kg IV every 3 weeks.

Key findings:

CHA.7.518.1.H4(S241P) was well tolerated through 10 mg/kg with no dose-limiting toxicities observed

The best timepoint response of stable disease (SD)/disease control rate reported in 9 of 13 patients (69%) with a median of seven prior anticancer therapies (range of 2-15).

All the patients with CRC (N=6) had microsatellite stable status, 5/6 pts (83%) had best timepoint response of stable disease.

Pharmacokinetic profile supports IV Q3 weekly dosing.

Peripheral PVRIG receptor occupancy greater than or equal to 90% was demonstrated at CHA.7.518.1.H4(S241P)>1 mg/kg.

There are 3 patients remaining on study treatment with CHA.7.518.1.H4(S241P) monotherapy.

Enrollment to CHA.7.518.1.H4(S241P) monotherapy dose at 20 mg/kg Q4 weekly is on-going.

### About the CHA.7.518.1.H4(S241P) Phase 1 Study

The Phase 1 open-label clinical trial of CHA.7.518.1.H4(S241P) was designed to assess the safety and tolerability of administering escalating doses of CHA.7.518.1.H4(S241P) monotherapy as well as of combination administration with Bristol-Myers Squibb's Opdivo® in patients with advanced solid tumors. Additionally, secondary endpoints include preliminary antitumor activity, pharmacokinetics and pharmacodynamics of CHA.7.518.1.H4(S241P) monotherapy as well as CHA.7.518.1.H4(S241P) in combination with Opdivo in patients with selected tumor types, including non-small cell lung cancer, ovarian cancer, breast cancer and endometrial cancer. The Phase 1 study, which is expected to enroll approximately 140 patients, is currently recruiting in the United States. Additional information is available at www.clinicaltrials.gov (NTC03667716).

### Example 7: Phase 1 Study of the Safety, Tolerability and Preliminary Anti-Tumor Activity of CHA.7.518.1.H4(S241P) Monotherapy in Patients with Advanced Solid Tumors

### Background

CHA.7.518.1.H4(S241P) is a novel first-in-class immune checkpoint inhibitor (ICI) of poliovirus receptor related immunoglobulin domain (PVRIG) discovered by Compugen's computational discovery program[1]. It inhibits the binding of PVRIG with its ligand, PVRL2.

PVRIG is a member of the DNAM/TIGIT signaling axis regulating the activity of TMK-cells

In preclinical experiments we have demonstrated that PVRIG inhibition leads to activation of T cells in the tumor microenvironment generating an anti-tumor immune response and tumor growth inhibition[2]

There is an urgent need to develop treatments for patients who are refractory or relapse after treatment with current ICIs

We hypothesized that CHA.7.518.1.H4(S241P) will be safe and tolerable and demonstrate preliminary antitumor activity as monotherapy in patients with advanced solid tumors

### Key Eligibility Criteria

Inclusion
Age ≥18 yrs
Histologically or cytologically confirmed, locally advanced or metastatic solid malignancy and has exhausted all the available standard therapy or is not a candidate for the available standard therapy
ECOG performance status 0-1
Prior immune checkpoint inhibitor permissible
Adequate hematological, hepatic and renal function

Exclusion
Symptomatic interstitial lung disease or inflammatory pneumonitis
Untreated or symptomatic central nervous system metastases
History of immune-related events that led to immunotherapy treatment discontinuation

### Results

No dose-limiting toxicities reported in the CHA.7.518.1.H4(S241P) dose ranges evaluated (0.01-10 mg/kg).

No treatment discontinuation due to adverse events were reported.

Majority of the TEAE were G1-2
Frequent TEAE were fatigue (46%), nausea (31%) and anxiety (23%)—all G1-2; disease progression G5 (23%)
Possible immune-related adverse events were rash (G1) and laboratory finding of elevated TSH (G1)

Serious adverse events were reported in 5/13 pts
In 3 pts, SAE were due to disease progression

All pts had stage IV disease at study entry and 8/13 (62%) had best response of PD to last prior therapy (ie refractory disease) before enrollment on this study
Best timepoint response of SD in 5/8 pts (63%), 1/5 pt with confirmed SD and 2 pts are ongoing on study treatment

Best timepoint response of SD/disease control rate reported in 9/13 pts (69%)

Colorectal cancer was the most common tumor type enrolled with 6/13 pts, all 6 pts had microsatellite stable status (MSS-CRC)
Disease control rate (SD) in 5/6 pts (83%) with CRC
Confirmed SD (week 12) in 4/6 pts (67%) with CRC
   Historical data 11% DCR and best timepoint response of SD at week 12 with pembrolizumab in pts with MSS-CRC [3]

All 3 enrolled pts with CRC-kras mutation had a best timepoint response of SD; 2/3 with confirmed SD

CHA.7.518.1.H4(S241P) exposure dose proportional with repeat dosing

Peripheral CHA.7.518.1.H4(S241P) receptor occupancy mg/kg IV Q3 weeks

### CONCLUSIONS

CHA.7.518.1.H4(S241P) well tolerated as monotherapy

Disease control rate—9/13 pts (69%)

Signal of antitumor activity in hard-to-treat MSS-CRC and pts with CRC with KRAS mutation
Confirmed SD in 4/6 pts (67%)—MSS-CRC
Confirmed SD in 2/3 pts with CRC-kras mutation

Signal of antitumor response in:
pts with prior treatment refractory disease
pts previously treated with ICI

Trend in dose-response relationship

CHA.7.518.1.H4(S241P) exposure dose-proportional permitting IV Q3 weeks dosing

Peripheral receptor occupancy at 90% with mg/kg CHA.7.518.1.H4(S241P) IV Q3 weeks

2 patients continue on study treatment

Study enrollment is ongoing in Arms A (CHA.7.518.1.H4(S241P) monotherapy) and B (CHA.7.518.1.H4(S241P) in combination with nivolumab)

Study NCT03667716 is in collaboration with Bristol-Myers Squibb

### REFERENCES

1. Whelan S, Ophir E, et al. PVRIG and PVRL2 Are Induced in Cancer and Inhibit CD8+ T-cell Function. Cancer Immunol Res. 2019 February; 7(2):257-268.
2. Murter B, Pan X, et al. Mouse PVRIG Has CD8+T Cell-Specific Contributory Functions and Dampens Antitumor Immunity. Cancer Immunol Res. 2019 February; 7(2):244-256.
3. Le D T, Uram J N, Wang H, et al. N Engl J Med. PD-1 Blockade in Tumors with Mismatch-Repair Deficiency. 2015 Jun. 25; 372(26):2509-20.

### Example 8: Phase 1 Study of CHA.7.518.1.H4(S241P) Monotherapy and in Combination with Nivolumab in Patients with Advanced Solid Tumors

### Background

CHA.7.518.1.H4(S241P) is a novel first-in-class humanized IgG4 monoclonal antibody that binds with high affinity to poliovirus receptor related immunoglobulin domain containing (PVRIG) blocking its interaction with its ligand, PVRL2 [1]

Nivolumab is an anti-PD-1 antibody approved in patients with several malignancies [2].

PD-1 inhibitors play an important role in this axis by modulating DNAM activation [3]

In preclinical experiments we have demonstrated that PVRIG inhibition alone and in combination with anti-PD-1 leads to activation of T cells in the tumor microenvironment generating an anti-tumor immune response and tumor growth inhibition [1]

Although ICI revolutionized cancer treatment there is an urgent need to develop treatments for patients who are refractory or relapse after treatment with ICI.

We hypothesize that CHA.7.518.1.H4(S241P) will be safe and tolerable and demonstrate antitumor activity in pts with R/R solid tumors

### Methods

NCT03667716 is an ongoing open-label first-in-human phase 1 study in pts with R/R solid tumors

We report on the initial part of this study evaluating the safety and tolerability of escalating doses of CHA.7.518.1.H4(S241P) monotherapy IV Q3 weeks and in combination with nivolumab 360 mg IV Q3 weeks.

### Primary Outcome Measures

To evaluate the safety profile of CHA.7.518.1.H4(S241P) as monotherapy and in combination with nivolumab in patients with advanced solid tumors

The incidence of adverse events and dose-limiting toxicities (21-day DLT window) graded as per CTCAE v4.03

To identify the maximum tolerated dose and/or the recommended dose for expansion

To characterize the PK profile of CHA.7.518.1.H4(S241P) as monotherapy and in combination with nivolumab

### Secondary Outcome Measures

To characterize the immunogenicity of CHA.7.518.1.H4(S241P) alone and in combination with nivolumab

To evaluate preliminary antitumor activity of CHA.7.518.1.H4(S241P) in combination with nivolumab (Phase 1b only) responses as per RECIST v1.1

### Exploratory Outcome Measures

To evaluate preliminary antitumor activity of CHA.7.518.1.H4(S241P) as monotherapy

To assess any association of DNAM axis members with clinical outcome

To explore evidence of CHA.7.518.1.H4(S241P)-mediated PD effect in blood as monotherapy as well as in combination with nivolumab

### Key Inclusion Criteria

Age ≥18 yrs

Histologically or cytologically confirmed, locally advanced or metastatic solid malignancy and has exhausted all the available standard therapy or is not a candidate for the available standard therapy

ECOG performance status 0-1

Prior anti-PD-1, anti-PD-L1, anti-CTLA-4, OX-40, CD137 permissible

Adequate hematological, hepatic and renal function

### Key Exclusion Criteria

Active autoimmune disease requiring systemic therapy in the last 2 years prior to the first dose of CHA.7.518.1.H4(S241P)

Symptomatic interstitial lung disease or inflammatory pneumonitis

Untreated or symptomatic central nervous system metastases

History of immune-related events that lead to immunotherapy treatment discontinuation

### Accrual Information

No dose-limiting toxicities have been observed in the 7th CHA.7.518.1.H4(S241P) monotherapy dose level and earlier dose levels (red box)

No dose-limiting toxicities have been observed in the 3rd CHA.7.518.1.H4(S241P)+nivolumab dose level and earlier dose levels (green box)

As of the date of this presentation the 8th CHA.7.518.1.H4(S241P) mono dose and 4th CHA.7.518.1.H4(S241P)+nivolumab dose levels are open to enrollment at IV Q4 weeks schedule

Study NCT03667716 is in collaboration with Bristol-Myers Squibb

### REFERENCE

Spencer L, Ofer L et al, Discovery of COM701, a therapeutic antibody targeting the novel immune checkpoint PVRIG, for the treatment of cancer. J Clin Oncol. 2017; (suppl; abstr 3074)
Nivolumab package insert. http://packageinserts.bms.com/pi/pi_opdivo.pdf Accessed Jul. 22, 2019.
Wang B, Zhang W et al., Combination cancer immunotherapy targeting PD-1 and GITR can rescue CD8+ T cell dysfunction and maintain memory phenotype. Sci. Immunol. 2018; Nov. 2:3(29).

### Example 9: CHA.7.518.1.H4(S241P) Demonstrates Antitumor Activity as Monotherapy and in Combination with Nivolumab in Patients with Advanced Malignancies

### Background

### Introduction

CHA.7.518.1.H4(S241P) is a novel first-in-class Immune checkpoint inhibitor (ICI) that binds with high affinity to poliovirus receptor related immunoglobulin domain containing (PVRIG) blocking its interaction with its ligand, PVRL2 and regulating the activity of T/NK cells through the DNAM/TIGIT axis. In preclinical experiments inhibition of PVRIG alone and in combination with anti-PD1 and/or TIGIT leads to tumor growth inhibition and activation of T-cells in the microenvironment generating an antitumor response.

### Methods:

A total of 28 pts (Arm A/B 16/12) with a variety of cancer types were enrolled (including patients with a variety of tumor types who had failed all available standard therapies). 16 patients in Arm A (CHA.7.518.1.H4(S241P) monotherapy dose escalation) and 12 patients in Arm B (CHA.7.518.1.H4(S241P) dose escalation with nivolumab). Hybrid accelerated (1st 4 dose cohorts in Arm A) and 3+3 study design (cohorts 5-8 in Arm A and all cohorts in Arm B). Patients with performance status ECOG 0-1 and advanced or metastatic solid tumors who failed standard of care treatment were eligible. Prior ICIs were permissible. In Arm A pts received CHA.7.518.1.H4(S241P) monotherapy 0.01, 0.03, 0.1, 0.3, 1, 3, 10 mg/kg (all IV Q3 weeks (wks)) and 20 mg/kg (IV Q4 wks). In Arm B, pts received CHA.7.518.1.H4(S241P) at 0.3, 1 or 3 mg/kg plus nivolumab 360 mg IV q3 wks (3 pts/dose cohort) and 3 pts received 10 mg/kg plus nivolumab 480 mg IV q4 wks. Treatment emergent adverse events (TEAEs) were reported per CTCAE v4.03 and responses per RECIST v1.1. Dose-limiting toxicities (DLTs) were evaluated within a 21-day or 28-day window (for 3- or 4-wks dosing schedule respectively). Data cutoff date was Jan. 23, 2020.

### Results:

The median number of prior anticancer therapies were: Arm A, 7 (range 2-15), Arm B, 5 (range 2-9). No DLTs have been reported in any of the dose cohorts. Treatment was well tolerated with no subjects discontinuing treatment due to toxicity, the most frequent TEAEs in Arm A were fatigue (46%), nausea (31%) and anxiety (23%)—all G1-2. In Arm B≥4 pts—anemia, lower extremity edema, rash and fatigue the majority being grade 1-2 (88%). In Arms A+B: partial response (PR)+stable disease (SD) was 57% (16/28). Of note: Arm A (CHA.7.518.1.H4(S241P) 20 mg/kg IV q4 wks): confirmed PR in apt with primary peritoneal cancer ongoing on treatment >15 weeks. Arm B: unconfirmed PR in apt with MSS-CRC on CHA.7.518.1.H4(S241P) 0.3 mg/kg plus nivolumab. A confirmed partial response in a patient with microsatellite stable primary peritoneal cancer enrolled in the eighth and last dose cohort in Arm A; the patient is continuing on study treatment (more than 15 weeks).

360 mg IV q3 wks, ongoing on treatment >34 wks.

Overall 11/28 patients remain on study treatment including 3 patients who have not reached first imaging assessment. For both treatment arms, the timepoint response of partial response and stable disease/disease control rate were reported in 16 of 28 patients (57%).

### Conclusion

CHA.7.518.1.H4(S241P) is well tolerated as monotherapy and in combination with nivolumab in a variety of heavily pretreated pts with advanced or metastatic solid tumors. CHA.7.518.1.H4(S241P) demonstrates encouraging preliminary antitumor activity with objective responses as monotherapy and in combination with nivolumab in hard to treat tumor types (primary peritoneal, microsatellite stable primary peritoneal cancer (MSS primary peritoneal cancer or MSS-PPC), and microsatellite stable colorectal cancer (MSS-CRC)).

### Example 10: CHA.7.518.1.H4(S241P) Demonstrates Antitumor Activity as Monotherapy and in Combination with Nivolumab in Patients with Advanced Malignancies

### Introduction

There is a high unmet medical need for the treatment of patients who are refractory to or relapse following treatment with checkpoint inhibitors.

Inhibition of poliovirus receptor related immunoglobulin domain containing (PVRIG) leads to enhanced activation of T and NK cells, and results in tumor growth inhibition in mouse tumor models (Spencer L, Ofer L et al, Discovery of COM701, a therapeutic antibody targeting the novel. immune checkpoint PVRIG, for the treatment of cancer. J Clin Oncol. 2017; (suppl; abstr 3074)).

CHA.7.518.1.H4(S241P) is a novel first-in-class humanized IgG4 monoclonal antibody that binds with high affinity PVRIG blocking its interaction with its ligand, PVRL2.

Previous data supported the preliminary antitumor activity of CHA.7.518.1.H4(S241P) monotherapy (Dumbrava E, Fleming G, Hamilton E et al. Journal for ImmunoTherapy of Cancer 2019, 7(Suppl 1):P421. SITC November 2019.)

The present data provides data related to the preliminary safety and antitumor activity of CHA.7.518.1.H4(S241P) in combination with nivolumab (Arm B) and we provide data update in CHA.7.518.1.H4(S241P) monotherapy dose cohorts (Arm A).

CHA.7.518.1.H4(S241P) well tolerated and with a manageable safety profile as monotherapy and in combination with nivolumab:
a. No increase in toxicity in combination with nivolumab.
b. No subjects discontinued study treatment due to toxicity of any study drug.

Single-agent MTD CHA.7.518.1.H4(S241P) 20 mg/kg IV Q4 wks; combination dose escalation continues.

Confirmed partial responses in 2 patients.

CHA.7.518.1.H4(S241P) monotherapy 20 mg/kg IV Q4 wks—primary peritoneal cancer (ongoing on study treatment 25 wks).

CHA.7.518.1.H4(S241P), (CHA.7.518.1.H4(S241P) 0.3 mg/kg IV Q3 weeks)+Nivolumab (480 mg IV q 3 wks)—MSS-CRC (ongoing on study treatment 44 wks).

Disease control rate for CHA.7.518.1.H4(S241P) monotherapy was 11/16 [69%] in diverse tumor types.

Disease control rate for CHA.7.518.1.H4(S241P)+nivolumab was 9/12 [75%] in diverse tumor types.

Durable stable disease (SD>6 months) in 6/28 pts and diverse tumor types.

Arm A (CHA.7.518.1.H4(S241P) monotherapy): Adenoid cystic CA, CRC-MSS.

Arm B (CHA.7.518.1.H4(S241P)+nivolumab): Anal SCC, CRC-MSS, Endometrial, NSCLC (squamous).

Preliminary CHA.7.518.1.H4(S241P) PK profile supports Q4 wks dosing.

CHA.7.518.1.H4(S241P) monotherapy dose expansion at RDFE planned (NSCLC, OVCA, Breast, Endometrial, MSS-CRC).

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed:

1. A method of treatment for cancer comprising administering nivolumab and an anti-PVRIG antibody, wherein said anti-PVRIG antibody is administered as a stable liquid pharmaceutical formulation and, wherein the stable liquid pharmaceutical formulation of the anti-PVRIG antibody comprises:
(a) an anti-PVRIG antibody, wherein said anti-PVRIG antibody comprises:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the vlCDR1, vlCDR2, and vlCDR3 from the light chain of CHA.7.518.1.H4(S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

2. The method of treatment according to any one of claim 1, wherein said anti-PVRIG antibody comprises a CH1-hinge-CH2-CH3 sequence of IgG4 (SEQ ID NO:17 or SEQ ID NO:50), wherein said hinge region optionally comprises mutations.

3. The method of treatment according to any one of claim 1 or 2, wherein said anti-PVRIG antibody comprises the CH1-hinge-CH2-CH3 region from IgG1, IgG2, IgG3, or IgG4, wherein said hinge region optionally comprises mutations.

4. The method of treatment according to any one of claims 1-3, wherein said heavy chain variable domain is from the heavy chain of CHA.7.518.1.H4(S241P) (SEQ ID NO:4) and said light chain variable domain is from the light chain of CHA.7.518.1.H4(S241P) (SEQ ID NO:9).

5. The method of treatment according to any one of claims 1-4, wherein said anti-PVRIG antibody comprises a CL region of human kappa 2 light chain.

6. The method of treatment according to any one of claims 1-5, wherein said pharmaceutical formulation comprises from 10 mM to 80 mM histidine, from 15 mM to 70 mM histidine, from 20 mM to 60 mM histidine, from 20 mM to 50 mM histidine, or from 20 mM to 30 mM histidine.

7. The method of treatment according to any one of claims 1-6, wherein said pharmaceutical formulation comprises about 25 mM histidine.

8. The method of treatment according to any one of claims 1-7, wherein said pharmaceutical formulation comprises from 30 mM to 100 mM NaCl, from 30 mM to 90 mM NaCl, from 40 mM to 80 mM NaCl, from 30 mM to 70 mM histidine, or from 45 mM to 70 mM NaCl.

9. The method of treatment according to any one of claims 1-8, wherein said pharmaceutical formulation comprises about 60 mM NaCl.

10. The method of treatment according to any one of claims 1-9, wherein said pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine.

11. The method of treatment according to any one of claims 1-10, wherein said pharmaceutical formulation comprises about 100 mM L-arginine.

12. The method of treatment according to any one of claims 1-11, wherein said pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, or from 0.009% to 0.05% w/v polysorbate 80.

13. The method of treatment according to any one of claims 1-12, wherein said pharmaceutical formulation comprises about 0.01% polysorbate 80.

14. The method of treatment according to any one of claims 1-13, wherein said pH is from 6 to 7.0.

15. The method of treatment according to any one of claims 1-14, wherein said pH is from 6.3 to 6.8.

16. The method of treatment according to any one of claims 1-15, wherein said pH is 6.5+/−0.2.

17. The method of treatment according to any one of claims 1-16, wherein said anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL.

18. The method of treatment according to any one of claims 1-17, wherein said formulation is stable at 2° C. to 8° C. for at least 1 week, 2 weeks, 3 weeks, 4 weeks, 5 weeks, 6 weeks, 7 weeks, 8 weeks, 9 weeks, or 10 weeks.

19. The method of treatment according to any one of claims 1-18, wherein said formulation is stable at about 20° C. to 25° C. for at least 1 week, 2 weeks, 3 weeks, 4 weeks, 5 weeks, or 6 weeks.

20. The method of treatment according to any one of claims 1-19, wherein said formulation is stable at 35° C. to 40° C. for at least 1 week, 2 weeks, 3 weeks, 4 weeks, or 5 weeks.

21. The method of treatment according to any one of claims 1-20, wherein said anti-PVRIG antibody is at a concentration of about 20 mg/mL.

22. The method of treatment according to any one of claims 1-21, wherein said anti-PVRIG antibody formulation comprises:
a) a heavy chain comprising:
i) a VH-CH1-hinge-CH2-CH3, wherein the VH is from CHA.7.518.1.H4(S241P) (SEQ ID NO:4) and wherein the CH1-hinge-CH2-CH3 region is from IgG4; and
b) a light chain comprising:
i) a VL-CL, wherein the VL from CHA.7.518.1.H4(S241P) (SEQ ID NO:9) and wherein the CL region is from human kappa 2 light chain.

23. The method of treatment according to claim 24, wherein said hinge region optionally comprises mutations.

24. The method of treatment of claim 23, wherein said hinge region optionally comprises mutations.

25. The method of treatment according to any one of claims 1-24, wherein said anti-PVRIG antibody formulation comprises:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13).

26. The method of treatment according to any one of claims 1-25, said anti-PVRIG antibody formulation comprising:
(a) an anti-PVRIG antibody, wherein said anti-PVRIG antibody comprises:
i) a heavy chain variable domain comprising the vhCDR1, vhCDR2, and vhCDR3 from the heavy chain of CHA.7.518.1.H4(S241P) (SEQ ID NO:4), and
ii) a light chain variable domain comprising the v1CDR1, v1CDR2, and v1CDR3 from the light chain of CHA.7.518.1.H4(S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

27. The method of treatment according to any one of claims 1-26, said anti-PVRIG antibody formulation comprising:
(a) an anti-PVRIG antibody, wherein said anti-PVRIG antibody comprises:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

28. The method of treatment according to any one of claims 1-27, wherein said anti-PVRIG antibody is administered at a dosage of about 0.01 mg/kg to about 20 mg/kg of the anti-PVRIG antibody or about 0.01 mg/kg to about 10 mg/kg of the anti-PVRIG antibody.

29. The method of treatment according to any one of claims 1-27, wherein said anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg of the anti-PVRIG antibody.

30. The method of treatment according to any one of claims 1-29, wherein said nivolumab is administered at a dosage of about 360 mg of nivolumab or 480 mg of nivolumab.

31. The method of treatment according to any one of claims 1-30, wherein said anti-PVRIG antibody is administered 20 mg/kg every 4 weeks.

32. The method of treatment according to any one of claims 1-31, wherein said cancer wherein said cancer selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, NSCLC, adenoid cystic cancer (including adenoid cystic carcinoma), primary peritoneal cancer, microsatellite stable primary peritoneal cancer, platinum resistant microsatellite stable primary peritoneal cancer, and/or Myelodysplastic syndromes (MDS).

33. The nivolumab and an anti-PVRIG antibody combination treatment according to any one of the method of treatment claims 1-32, for use in a method of treating cancer.

34. The use according to claim 33, wherein said cancer wherein said cancer selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, NSCLC, adenoid cystic cancer (including adenoid cystic carcinoma), primary peritoneal cancer, microsatellite stable primary peritoneal cancer, platinum resistant microsatellite stable primary peritoneal cancer, and/or Myelodysplastic syndromes (MDS).
